SEQUENCE LISTING (52 sequences)

SEQ ID NO: 1 - protein, 1748 aa, Homo sapiens
MSARAAAAKSTAMEETAIWEQHTVTLHRAPGFGFGIAISGGRDNPHFQSGETSIVISDVLKGGPAEGQLQENDRVAMVNGVSMDNVEHAFAVQQLRKSGKNAKITIRRKKKVQIPVSRPDPEPVSDNEEDSYDEEIHDPRSGRSGVVNRRSEKIWPRDRSASRERSLSPRSDRRSVASSQPAKPTKVTLVKSRKNEEYGLRLASHIFVKEISQDSLAARDGNIQEGDVVLKINGTVTENMSLTDAKTLIERSKGKLKMVVQRDERATLLNVPDLSDSIHSANASERDDISEIQSLASDHSGRSHDRPPRRSRSRSPDQRSEPSDHSRHSPQQPSNGSLRSRDEERISKPGAVSTPVKHADDHTPKTVEEVTVERNEKQTPSLPEPKPVYAQVGQPDVDLPVSPSDGVLPNSTHEDGILRPSMKLVKFRKGDSVGLRLAGGNDVGIFVAGVLEDSPAAKEGLEEGDQILRVNNVDFTNIIREEAVLFLLDLPKGEEVTILAQKKKDVYRRIVESDVGDSFYIRTHFEYEKESPYGLSFNKGEVFRVVDTLYNGKLGSWLAIRIGKNHKEVERGIIPNKNRAEQLASVQYTLPKTAGGDRADFWRFRGLRSSKRNLRKSREDLSAQPVQTKFPAYERVVLREAGFLRPVTIFGPIADVAREKLAREEPDIYQIAKSEPRDAGTDQRSSGIIRLHTIKQIIDQDKHALLDVTPNAVDRLNYAQWYPIVVFLNPDSKQGVKTMRMRLCPESRKSARKLYERSHKLRKNNHHLFTTTINLNSMNDGWYGALKEAIQQQQNQLVWVSEGKADGATSDDLDLHDDRLSYLSAPGSEYSMYSTDSRHTSDYEDTDTEGGAYTDQELDETLNDEVGTPPESAITRSSEPVREDSSGMHHENQTYPPYSPQAQPQPIHRIDSPGFKPASQQKAEASSPVPYLSPETNPASSTSAVNHNVNLTNVRLEEPTPAPSTSYSPQADSLRTPSTEAAHIMLRDQEPSLSSHVDPTKVYRKDPYPEEMMRQNHVLKQPAVSHPGHRPDKEPNLTYEPQLPYVEKQASRDLEQPTYRYESSSYTDQFSRNYEHRLRYEDRVPMYEEQWSYYDDKQPYPSRPPFDNQHSQDLDSRQHPEESSERGYFPRFEEPAPLSYDSRPRYEQAPRASALRHEEQPAPGYDTHGRLRPEAQPHPSAGPKPAESKQYFEQYSRSYEQVPPQGFTSRAGHFEPLHGAAAVPPLIPSSQHKPEALPSNTKPLPPPPTQTEEEEDPAMKPQSVLTRVKMFENKRSASLETKKDVNDTGSFKPPEVASKPSGAPIIGPKPTSQNQFSEHDKTLYRIPEPQKPQLKPPEDIVRSNHYDPEEDEEYYRKQLSYFDRRSFENKPPAHIAASHLSEPAKPAHSQNQSNFSSYSSKGKPPEADGVDRSFGEKRYEPIQATPPPPPLPSQYAQPSQPVTSASLHIHSKGAHGEGNSVSLDFQNSLVSKPDPPPSQNKPATFRPPNREDTAQAAFYPQKSFPDKAPVNGTEQTQKTVTPAYNRFTPKPYTSSARPFERKFESPKFNHNLLPSETAHKPDLSSKTPTSPKTLVKSHSLAQPPEFDSGVETFSIHAEKPKYQINNISTVPKAIPVSPSAVEEDEDEDGHTVVATARGIFNSNGGVLSSIETGVSIIIPQGAIPEGVEQEIYFKVCRDNSILPPLDKEKGETLLSPLVMCGPHGLKFLKPVELRLPHCDPKTWQNKCLPGDPNYLVGANCVSVLIDHF

SEQ ID NO: 2 - DNA, 7888 bp, Homo sapiens
tccgggtatggatgtcaatcttttgtctacaatgtgaatacatttatccttcggggaccatcaagactttcaggaaaggccccgcctgtctctgcgcggccactttgctgggacaaaggtcaactgaagaagtgggcaggcccgaggcaggagagatgctgaggagtccatgtgcaggggagggaaagggagaggcagtcagggagaggaggaggaggtaccgccagaaggggatcctcccgctccgaaaaccagacaccgggtcttgccctgtggtccaggcaggagtgcagtggtgcaacctcagctcactgcagccttgacctccccgggctcaagcgatcctccggccacagcacttggctgttcagcggctggaggagcagggccccaggtcctccccaccctcacctgctgctcccaggtcgtggccgtcttgctcttccaggtccttctctagggatgcaatattcacattgctaagatgcaggtctaacgcagaacctgtcaacagagccccccatcatccacagcccacccagcgctgcagagctcaggaagcctagctgaggaggacgaccgtcccacctgggcttagagtgagaccaagggcagaaggcgtgggagttgctggggcagccagggaaggacacccccagcccgtcctcgcagccccccacaggcagtgggaggcttggctgttcctccggcaaaacgggcatgctcagtgggccgggccggcaggtttgcgtggccgctgagttgccggcgccggctgagccagcggacgccgcgttccttggcggccgccggttcccgggaagttacgtggcgaagccggcttccgaggagacgccgggaggccacgggtgctgctgacgggcgggcgaccgggcgaggccgacgtggccgggctgcgaaagctgcgggaggccgagtgggtgaccgcgctcggagggaggtgccggtcgggcgcgccccgtggagaagacccgggcggggcgggcgcttcccggacttttgtccgagttgaattccctccccctgggccgggcccttccgtccgcccccgcccgtgccccgctcgctctcgggagatgtttatttgggctgtggcgtgaggagcgggcgggccagcgccgcggagtttcgggtccgaggagcctcgcgcggcgctggagagagacaagatgtccgccagagctgcggccgccaagagcacagcaatggaggaaacagctatatgggaacaacatacagtgacgcttcacagggctcctggatttggatttggaattgcaatatctggtggacgagataatcctcattttcagagtggggaaacgtcaatagtgatttcagatgtgctgaaaggaggaccagctgaaggacagctacaggaaaatgaccgagttgcaatggttaacggagtttcaatggataatgttgaacatgcttttgctgttcagcaactaaggaaaagtgggaaaaatgcaaaaattacaattagaaggaagaagaaagttcaaataccagtaagtcgtcctgatcctgaaccagtatctgataatgaagaagatagttatgatgaggaaatacatgatccaagaagtggccggagtggtgtggttaacagaaggagtgagaagatttggccgagggatagaagtgcaagtagagagaggagcttgtccccgcggtcagacaggcggtcagtggcttccagccagcctgctaaacctactaaagtcacactggtgaaatcccggaaaaatgaagaatatggtcttcgattggcaagccatatatttgttaaggaaatttcacaagatagtttggcagcaagagatggcaatattcaagaaggtgatgttgtattgaagataaatggtactgtgacagaaaatatgtcattgacagatgcaaagacattgatagaaaggtctaaaggcaaattaaaaatggtagttcaaagagatgaacgggctacgctattgaatgtccctgatctttctgacagcatccactctgctaatgcctctgagagagacgacatttcagaaattcagtcactggcatcagatcattctggtcgatcacacgataggcctccccgccgcagccggtcacgatctcctgaccagcggtcagagccttctgatcattccaggcactcgccgcagcagccaagcaatggcagtctccggagtagagatgaagagagaatttctaaacctggggctgtctcaactcctgtaaagcatgctgatgatcacacacctaaaacagtggaagaagttacagttgaaagaaatgagaaacaaacaccttctcttccagaaccaaagcctgtgtatgcccaagttggcaaccagatgtggatttacctgtcagtccatctgatggtgtcctacctaattcaactcatgaagatgggatttcttcggcccagcatgaaattggtaaaattcagaaaaggagatagtgtgggtttgcggctggctggtggaaatgatgttggaatatttgtagctggcgttctagaagatagccctgcagccaaggaaggcttagaggaaggtgatcaaattctcagggtaaacaacgtagattttacaaatatcataagagaagaagccgtccttttcctgcttgacctccctaaaggagaagaagtgaccatattggctcagaagaagaaggatgtttatcgtcgcattgtagaatcagatgtaggagattctttctatattagaacccattttgaatatgaaaaggaatctccctatggacttagttttaacaaaggagaggtgttccgtgctgtggataccttgtacaatggaaaactgggctcttggcttgctattcgaattggtaaaaatcataaggaggtagaacgaggcatcatccctaataagaacagagctgagcagctagccagtgtacagtatacacttccaaaaacagcaggcggagaccgtgctgacttctggagattcagaggtcttcgcagctccaagagaaatcttcgaaaaagcagagaggatttgtccgctcagcctgttcaaacaaagtttccagcttatgaaagagtggttcttcgagaagctggatttctgaggcctgtaaccatttttggaccaatagctgatgttgccagagaaaagctggcaagagaagaaccagatatttatcaaattgcaaagagtgaaccacgagacgctggaactgaccaacgtagctctggctatattcgcctgcatacaataaagcaaatcatagatcaagacaaacatgctttattagatgtaacaccaaatgcagttgatcgtcttaactatgcccagtggtatccaattgttgtatttcttaaccctgattctaagcaaggagtaaaaacaatgagaatgaggttatgtccagaatctcggaaaagtgccaggaagttatacgagcgatctcataaacttgctaaaaataatcaccatctttttacaactacaattaacttaaattcaatgaatgatggttggtatggtgcgctgaaagaagcagttcaacaacagcaaaaccagctggtatgggtttccgagggaaaggcggatggtgctacaagtgatgaccttgatttgcatgatgatcgtctgtcctacctgtcagctccaggtagtgaatactcaatgtatagcacggacagtagacacacttctgactatgaagacacagacacagaaggcggggcctacactgatcaagaactagatgaaactcttaatgatgaggttgggactccaccggagtctgccattacacggtcctctgagcctgtaagagaggactcctctggaatgcatcatgaaaaccaaacatatcctccttactcaccacaagcgcagccacaaccaattcatagaatagactcccctggatttaagccagcctctcaacagaaagcagaagcttcatctccagtcccttacctttcgcctgaaacaaacccagcatcatcaacctctgctgttaatcataatgtaaatttaactaatgtcagactggaggagcccaccccagctccttccacctcttactcaccacaagctgattctttaagaacaccaagtactgaggcagctcacataatgctaagagatcaagaaccatcattgtcgtcgcatgtagatccaacaaaggtgtatagaaaggatccatatcccgaggaaatgatgaggcagaaccatgttttgaaacagccagccgttagtcacccagggcacaggccagacaaagagcctaatctgacctatgaaccccaactcccatacgtagagaaacaagccagcagagacctcgagcagcccacatacagatacgagtcctcaagctatacggaccagttttctcgaaactatgaacatcgtctgcgatacgaagatcgcgtccccatgtatgaagaacagtggtcatattatgatgacaaacagccctacccatctcggccaccttttgataatcagcactctcaagaccttgactccagacagcatcccgaagagtcctcagaacgagggtactttccacgttttgaagagccagcccctctgtcttacgacagcagaccacgttacgaacaggcacctagagcatccgccctgcggcacgaagagcagccagctcctgggtatgacacacatggtagactcagaccggaagcccagccccacccttcagcagggcccaagcctgcagagtccaagcagtattttgagcaatattcacgcagttacgagcaagtaccaccccaaggatttacctctagagcaggtcattttgagcctctccatggtgctgcagctgtccctccgctgataccttcatctcagcataagccagaagctctgccttcaaacaccaaaccactgcctccacccccaactcaaaccgaagaagaggaagatccagcaatgaagccacagtctgtactcaccagagttaagatgtttgaaaacaaaagatctgcatccttagagaccaagaaggatgtaaatgacactggcagttttaagcctccagaagtagcatctaaaccttcaggtgctcccatcattggtcccaaacccacttctcagaatcaattcagtgaacatgacaaaactctgtacaggatcccagaacctcaaaaacctcaactgaagccacctgaagatattgttcggtccaatcattatgaccctgaagaagatgaagaatattatcgaaaacagctgtcatactttgaccgaagaagttttgagaataagcctcctgcacacattgctgccagccatctctccgagcctgcaaagccagctcattctcagaatcaatcaaatttttctagttattcttcaaagggaaagcctcctgaagctgatggtgtggatagatcatttggcgagaaacgctatgaacccatccaggccactccccctcctcctccattgccctcgcagtatgcccagccatctcagcctgtcaccagcgcgtctctccacatacattctaagggagcacatggtgaaggtaattcagtgtcattggattttcagaattccttagtgtccaaaccagacccacctccatctcagaataagccagcaactttcagaccaccaaaccgagaagatactgctcaggcagctttctatccccagaaaagttttccagataaagccccagttaatggaactgaacagactcagaaaacagtcactccagcatacaatcgattcacaccaaaaccatatacaagttctgcccgaccatttgaacgcaagtttgaaagtcctaaattcaatcacaatcttctgccaagtgaaactgcacataaacctgacttgtcttcaaaaactcccacttctccaaaaactcttgtgaaatcgcacagtttggcacagcctcctgagtttgacagtggagttgaaactttctctatccatgcagagaagcctaaatatcaaataaataatatcagcacagtgcctaaagctattcctgtgagtccttcagctgtggaagaggatgaagatgaagatggtcatactgtggtggccacagcccgaggcatatttaacagcaatgggggcgtgctgagttccatagaaactggtgttagtataattatccctcaaggagccattcccgaaggagttgagcaggaaatctatttcaaggtctgccgggacaacagcatccttccacctttagataaagagaaaggtgaaacactgctgagtcctttggtgatgtgtggtccccatggcctcaagttcctgaagcctgtggagctgcgcttaccacactgtgatcctaaaacctggcaaaacaagtgtcttcccggagatccaaattatctcgttggagcaaactgtgtttctgtccttattgaccacttttaactcttgaaatataggaacttaaataatgtgaaactggattaaacttaatctaaatggaaccactctatcaagtattataccttttttagagttgatactacagtttgttagtatgaggcatttgtttgaactgataaagatgagtgagcatgcccctgaaccatggtcggaaaacatgctacacactgcatgtttgtgattgacgggactgttggtattggctagaggttcaaagatattttgctttgtgatttttgtaatttttttatcgtcactgcttaacttcacatattgatttccgttaaaataccagccagtaaatgggggtgcatttgaggtctgttctttccaaagtacactgtttcaaactttactatggccctggcctagcatacgtacacattttattttattatgcatgaagtaatatgcacacattttttaaatgcacctggaatatataaccagtgttgtggatttaacagaaatgtacagcaaggagatttacaactgggggagggtgaagtgaagacaatgacttactgtacatgaaaacacatttttcttagggaaggatacaaaagcatgtgagactggttccatggcctcttcagatctctaacttcaccatattaccacagacatactaaccagcagaaatgccttaccctcatgttcttaattcttagctcattctccttgtgttactaagtttttatggcttttgtgcattatctagatactgtatcatgacaaagactgagtacgttgtgcatttggtggtttcagaaatgtgttatcacccagaagaaaatagtggtgtgatttggggatatttttttcttttcttttcttttcttttttttttttttttgacaaggggcagtggtggttttctgttctttctggctatgcatttgaaaattttgatgttttaaggatgcttgtacataatgcgtgcataccacttttgttcttggtttgtaaattaacttttataaactttaccttttttatacataaacaagaccacgtttctaaaggctacctttgtattctctcctgtacctcttgagccttgaactttgacctctgcagcaataaagcagcgtttctatgacacatgcaaggtcattttttttaagaaaaaggatgcacagagttgttacatttttaagtgctgcatttaaaagatacagttactcagaattctctagtttgattaaattcttgcaaagtatccctactgtaatttgtgatacaatgctgtgccctaaagtgtatttttttactaatagacaatttattatgacacatcagcacgatttctgtttaaataatacaccactacattctgttaatcattaggtgtgactgaatttcttttgccgttattaaaaatctcaaatttctaaatctccaaaataaaactttttaaaataaaaaaaaat

SEQ ID NO: 3 - protein, 1190 aa, Homo sapiens
MPVRGDRGFPPRRELSGWLRAPGMEELIWEQYTVTLQKDSKRGFGIAVSGGRDNPHFENGETSIVISDVLPGGPADGLLQENDRVVMVNGTPMEDVLHSFAVQQLRKSGKVAAIVVKRPRKVQVAALQASPPLDQDDRAFEVMDEFDGRSFRSGYSERSRLNSHGGRSRSWEDSPERGRPHERARSRERDLSRDRSRGRSLERGLDQDHARTRDRSRGRSLERGLDHDFGPSRDRDRDRSRGRSIDQDYERAYHRAYDPDYERAYSPEYRRGARHDARSRGPRSRSREHPHSRSPSPEPRGRPGPIGVLLMKSRANEEYGLRLGSQIFVKEMTRTGLATKDGNLHEGDIILKINGTVTENMSLTDARKLIEKSRGKLQLVVLRDSQQTLINIPSLNDSDSEIEDISEIESNRSFSPEERRHQYSDYDYHSSSEKLKERPSSREDTPSRLSRMGATPTPFKSTGDIAGTVVPETNKEPRYQEDPPAPQPKAAPRTFLRPSPEDEAIYGPNTKMVRFKKGDSVGLRLAGGNDVGIFVAGIQEGTSAEQEGLQEGDQILKVNTQDFRGLVREDAVLYLLEIPKGEMVTILAQSRADVYRDILACGRGDSFFIRSHFECEKETPQSLAFTRGEVFRVVDTLYDGKLGNWLAVRIGNELEKGLIPNKSRAEQMASVQNAQRDNAGDRADFWRMRGQRSGVKKNLRKSREDLTAVVSVSTKFPAYERVLLREAGFKRPVVLFGPIADIAMEKLANELPDWFQTAKTEPKDAGSEKSTGVVRLNTVRQIIEQDKHALLDVTPKAVDLLNYTQWFPIVIFFNPDSRQGVKTMRQRLNPTSNKSSRKLFDQANKLKKTCAHLFTATINLNSANDSWFGSLKDTIQHQQGEAVWVSEGKMEGMDDDPEDRMSYLTAMGADYLSCDSRLISDFEDTDGEGGAYTDNELDEPAEEPLVSSITRSSEPVQHEESIRKPSPEPRAQMRRAASSDQLRDNSPPPAFKPEPPKAKTQNKEESYDFSKSYEYKSNPSAVAGNETPGASTKGYPPPVAAKPTFGRSILKPSTPIPPQEGEEVGESSEEQDNAPKSVLGKVKIFEKMDHKARLQRMQELQEAQNARIEIAQKHPDIYAVPIKTHKPDPGTPQHTSSRPPEPQKAPSRPYQDTRGSYGSDAEEEEYRQQLSEHSKRGYYGQSARYRDTEL

SEQ ID NO: 4 - DNA, 4484 bp, Homo sapiens
tgcccaggaggagtaggagcaggagcagaagcagaagcggggtccggagctgcgcgcctacgcgggacctgtgtccgaaatgccggtgcgaggagaccgcgggtttccaccccggcgggagctgtcaggttggctccgcgccccaggcatggaagagctgatatgggaacagtacactgtgaccctacaaaaggattccaaaagaggatttggaattgcagtgtccggaggcagagacaacccccactttgaaaatggagaaacgtcaattgtcatttctgatgtgctcccgggtgggcctgctgatgggctgctccaagaaaatgacagagtggtcatggtcaatggcacccccatggaggatgtgcttcattcgtttgcagttcagcagctcagaaaaagtgggaaggtcgctgctattgtggtcaagaggccccggaaggtccaggtggccgcacttcaggccagccctcccctggatcaggatgaccgggcttttgaggtgatggacgagtttgatggcagaagtttccggagtggctacagcgagaggagccggctgaacagccatggggggcgcagccgcagctgggaggacagcccggaaagggggcgtccccatgagcgggcccggagccgggagcgggacctcagccgggaccggagccgtggccggagcctggagcggggcctggaccaagaccatgcgcgcacccgagaccgcagccgtggccggagcctggagcggggcctggaccacgactttgggccatcccgggaccgggaccgtgaccgcagccgcggccggagcattgaccaggactacgagcgagcctatcaccgggcctacgacccagactacgagcgggcctacagcccggagtacaggcgcggggcccgccacgatgcccgctctcggggaccccgaagccgcagccgcgagcacccgcactcacggagccccagccccgagcctagggggcggccggggcccatcggggtcctcctgatgaaaagcagagcgaacgaagagtatggtctccggcttgggagtcagatcttcgtaaaggaaatgacccgaacgggtctggcaactaaagatggcaaccttcacgaaggagacataattctcaagatcaatgggactgtaactgagaacatgtctttaacggatgctcgaaaattgatagaaaagtcaagaggaaaactacagctagtggtgttgagagacagccagcagaccctcatcaacatcccgtcattaaatgacagtgactcagaaatagaagatatttcagaaatagagtcaacccgatcattttctccagaggagagacgtcatcagtattctgattatgattatcattcctcaagtgagaagctgaaggaaaggccaagttccagagaggacacgccgagcagattgtccaggatgggtgcgacacccactccctttaagtccacaggggatattgcaggcacagttgtcccagagaccaacaaggaacccagataccaagaggaacccccagctcctcaaccaaaagcagccccgagaacttttcttcgtcctagtcctgaagatgaagcaatatatggccctaataccaaaatggtaaggttcaagaagggagacagcgtgggcctccggttggctggtggcaatgatgtcgggatatttgttgctggcattcaagaagggacctcggcggagcaggagggccttcaagaaggagaccagattctgaaggtgaacacacaggatttcagaggattagtgcgggaggatgccgttctctacctgttagaaatccctaaaggtgaaatggtgaccattttagctcagagccgagccgatgtgtatagagacatcctggcttgtggcagaggggattcgttttttataagaagccactttgaatgtgagaaggaaactccacagagcctggccttcaccagaggggaggtcttccgagtggtagacacactgtatgacggcaagctgggcaactggctggctgtgaggattgggaacgagttggagaaaggcttaatccccaacaagagcagagctgaacaaatggccagtgttcaaaatgcccagagagacaacgctggggaccgggcagatttctggagaatgcgtggccagaggtctggggtgaagaagaacctgaggaaaagtcgggaagacctcacagctgttgtgtctgtcagcaccaagttcccagcttatgagagggttttgctgcgagaagctggtttcaagagacctgtggtcttattcggccccatagctgatatagcaatggaaaaattggctaatgagttacctgactggtttcaaactgctaaaacggaaccaaaagatgcaggatctgagaaatccactggagtggtccggttaaataccgtgaggcaagttattgaacaggataagcatgcactactggatgtgactccgaaagctgtggacctgttgaattacacccagtggttctcaattgtgatttctttcacgccagactccagacaaggtgtcaacaccatgagacaaaggttagacccaacgtccaacaatagttctcgaaagttatttgatcacgccaacaagcttaaaaaaacgtgtgcacacctttttacagctacaatcaacctaaattcagccaatgatagctggtttggcagcttaaaggacactattcagcatcagcaaggagaagcggtttgggtctctgaaggaaagatggaagggatggatgatgaccccgaagaccgcatgtcctacttaactgccatgggcgcagactatctgagttgcgacagccgcctcatcagtgactttgaagacacggacggtgaaggaggcgcctacactgacaatgagctggatgagccagccgaggagccgctggtgtcgtccatcacccgctcctcggagccggtgcagcacgaggagagcataaggaaacccagcccagagccacgagctcagatgaggagggctgctagcagcgatcaacttagggacaatagcccgcccccagcattcaagccagagccgtccaaggccaaaacccagaacaaagaagaatcctatgacttctccaaatcctatgaatataagtcaaacccctctgccgttgctggtaatgaaactcctggggcatctaccaaaggttatcctcctcctgttgcagcaaaacctacctttgggcggtctatactgaagccctccactcccatccctcctcaagagggtgaggaggtgggagagagcagtgaggagcaagataatgctcccaaatcagtcctgggcaaagtcaaaatatttggagaagatggatcacaagggccagggttacaagagaatgcaggagctccaggaagcacagaatgcaaggatcgaaattgcccagaagcatcctgatatctatgcagttccaatcaaaacgcacaagccagaccctggcacgccccagcacacgagttccagaccccctgagccacagaaagctccttccagaccttatcaggataccagaggaagttatggcagtgatgccgaggaggaggagtaccgccagcagctgtcagaacactccaagcgcggttactatggccagtctgcccgataccgggacacagaattatagatgtctgagcacggactctcccaggcctgcctgcatggcatcagactagccactcctgccaggccgccgggatggttcttctccagttagaatgcaccatggagacgtggtgggactccagctcgtgtgtcctcatggagaacccaggggacagctggtgcaaattcagaactgagggctctgtttgtgggactgggttagaggagtctgtggctttttgttcagaattaagcagaacactgcagtcagatcctgttacttgcttcagtggaccgaaatctgtattctgtttgcgtacttgtaatatgtatattaagaagcaataactatttttcctcattaatagctgccttcaaggactgtttcagtgtgagtcagaatgtgaaaaaggaataaaaaatactgttgggctcaaactaaattcaaagaagtactttattgcaactcttttaagtgccttggatgagaagtgtcttaaattttcttcctttgaagctttaggcagagccataatggactaaaacattttgactaagtttttataccagcttaatagctgtagttttccctgcactgtgtcatcttttcaaggcatttgtctttgtaatattttccataaatttggactgtctatatcataactatacttgatagtttggctataagtgctcaatagcttgaagcccaagaagttggtatcgaaatttgttgtttgtttaaacccaagtgctgcacaaaagcagatacttgaggaaaacactatttccaaaagcacatgtattgacaacagttttataatttaataaaaaggaatacattgcaatccgt

SEQ ID NO: 5 - protein, 933 aa, Homo sapiens [sequence omitted]

SEQ ID NO: 6 - DNA, 3075 bp, Homo sapiens
agttccactggcaggcgacctgcctccctgttgccaccacaagagaggaaaagttggtcaaacaggtggggaggccagagctacaagcctcgggttccctccccaccacccgtgccaggcaggcacccgggccctggcacctgctgcctgcccagaggccacccagcctcctagacaggtggctgacatggaggagctgaccatctgggaacagcacacggccacactgtccaaggacccccgccggggctttggcattgcgatctctggaggccgagaccggcccggtggatccatggttgtatctgacgtggtacctggagggccggcggagggcaggctacagacaggcgaccacattgtcatggtgaacggggtttccatggagaatgccacctccgcgtttgccattcagatactcaagacctgcaccaagatggccaacatcacagtgaaacgtccccggaggatcctcctgcccgccaccaaagccagcccctccagcccagggcgccaggactcggatgaagacgatgggccccagcgggtggaggaggtggaccagggccggggctatgacggcgactcatccagtggctccggccgctcctgggacgagcgctcccgccggccgaggcctggtcgccggggccgggccggcagccatgggcgtaggagcccaggtggtggctctgaggccaacgggctggccctggtgtccggctttaagcggctgccacggcaggacgtgcagatgaagcctgtgaagtcagtgctggtgaagaggagagacagcgaagagtttggcgtcaagctgggcagtcagatcttcatcaagcacattacagattcgggcctggctgcccggcaccgtgggctgcaggaaggagatctcattctacagatcaacggggtgtctagccagaacctgtcactgaacgacacccggcgactgattgagaagtcagaagggaagctaagcctgctggtgctgagagatcgtgggcagttcctggtgaacattccgcctgctgtcagtgacagcgacagctcgccattggaggacatctcggacctcgcctcggagctatcgcaggcaccaccatcccacatcccaccaccaccccggcatgctcagcggagccccgaggccagccagaccgactctcccgtggagagtccccggcttcggcgggaaagttcagtagattccagaaccatctcggaaccagatgagcaacggtcagagttgcccagggaaagcagctatgacatctacagagtgcccagcagtcagagcatggaggatcgtgggtacagccccgacacgcgtgtggtccgcttcctcaagggcaagagcatcgggctgcggctggcagggggcaatgacgtgggcatcttcgtgtccggggtgcaggcgggcagcccggccgacgggcagggcatccaggagggagatcagattctgcaggtgaatgacgtgccattccagaacctgacacgggaggaggcagtgcagttcctgctggggctgccaccaggcgaggagatggagctggtgacgcagaggaagcaggacattttctggaaaatggtgcagtcccgcgtgggtgactccttctacatccgcactcactttgagctggagcccagtccaccgtctggcctgggcttcacccgtggcgacgtcttccacgtgctggacacgctgcaccccggccccgggcagagccacgcacgaggaggccactggctggcggtgcgcatgggtcgtgacctgcgggagcaagagcggggcatcattcccaaccagagcagggcggagcagctggccagcctggaagctgcccagagggccgtgggagtcgggcccggctcctccgcgggctccaatgctcgggccgagttctggcggctgcggggtcttcgtcgaggagccaagaagaccactcagcggagccgtgaggacctctcagctctgacccgacagggccgctacccgccctacgaacgagtggtgttgcgagaagccagtttcaagcgcccggtagtgatcctgggacccgtggccgacattgctatgcagaagttgactgctgagatgcctgaccagtttgaaatcgcagagactgtgtccaggaccgacagcccctccaagatcatcaaactagacaccgtgcgggtgattgcagaaaaagacaagcatgcgctcctggatgtgaccccctccgccatcgagcgcctcaactatgtgcagtactaccccattgtggtcttcttcatccccgagagccggccggccctcaaggcactgcgccagtggctggcgcctgcctcccgccgcagcacccgtcgcctctacgcacaagcccagaagctgcgaaaacacagcagccacctcttcacagccaccatccctctgaatggcacgagtgacacctggtaccaggagctcaaggccatcattcgagagcagcagacgcggcccatctggacggcggaagatcagctggatggctccttggaggacaacctagacctccctcaccacggcctggccgacagctccgctgacctcagctgcgacagccacgttaacagcgactacgagacggacggcgagggcggcgcgtacacggatggcgagggctacacagacggcgagggggggccctacacggatgtggatgatgagcccccggctccagccctggcccggtcctcggagcccgtgcaggcagatgagtcccagagcccgagggatcgtgggagaatctcggctcatcagggggcccaggtggacagccgccacccccagggacagtggcgacaggacagcatgcgaacctatgaacgggaagccctgaagaaaaagtttacgcgagtccgtgatgcggagtcctccgatgaagacggctatgactggggtccggccactgacctgtgacctctcgcaggctgccagctggtccgtcctccttctccctccctggggctgggactcagtttcccatacagaacccacaaccttacctccctccgcctggtctttaataaacagagtattttcacagc

SEQ ID NO: 7 - protein, 522 aa, Homo sapiens
MSSRPLESPPPYRPDEFKPNHYAPSNDIYGGEMHVRPMLSQPAYSFYPEDEILHFYKWTSPPGVIRILSMLIIVMCIAIFACVASTLAWDRGYGTSLLGGSVGYPYGGSGFGSYGSGYGYGYGYGYGYGGYTDPRAAKGFMLAMAAFCFIAALVIFVTSVIRSEMSRTRRYYLSVIIVSAILGIMVFIATIVYIMGVNPTAQSSGSLYGSQIYALCNQFYTPAATGLYVDQYSYHYCVVDPQEAIAIVLGFMIIVAFALIIFFAVKTRRKMDRYDKSNILWDKEHIYDEQPPNVEEWVKNVSAGTQDVPSPPSDYVERVDSPMAYSSNGKVNDKRFYPESSYKSTPVPEVVQELPLTSPVDDFRQPRYSSGGNFETPSKRAPAKGRAGRSKRTEQDHYETDYTTGGESCDELEEDWIREYPPITSDQQRQLYKRNFDTGLQEYKSLQSELDEINKELSRLDKELDDYREESEEYMAAADEYNRLKQVKGSADYKSKKNHCKQLKSKLSHIKKMVGDYDRQKT

SEQ ID NO: 8 - DNA, 2379 bp, Homo sapiens
ctcccgcgtccacctctccctccctgcttcctctggcggaggcggcaggaaccgagagccaggtccagagcgccgaggagccggtctaggacgcagcagattggtttatcttggaagctaaagggcattgctcatcctgaagatcagctgaccattgacaatcagccatgtcatccaggcctcttgaaagtccacctccttacaggcctgatgaattcaaaccgaatcattatgcaccaagcaatgacatatatggtggagagatgcatgttcgaccaatgctctctcagccagcctactctttttacccagaagatgaaattcttcacttctacaaatggacctctcctccaggagtgattcggatcctgtctatgctcattattgtgatgtgcattgccatctttgcctgtgtggcctccacgcttgcctgggacagaggctatggaacttcccttttaggaggtagtgtaggctacccttatggaggaagtggctttggtagctacggaagtggctatggctatggctatggttatggctatggctacggaggctatacagacccaagagcagcaaagggcttcatgttggccatggctgccttttgtttcattgccgcgttggtgatctttgttaccagtgttataagatctgaaatgtccagaacaagaagatactacttaagtgtgataatagtgagtgctatcctgggcatcatggtgtttattgccacaattgtctatataatgggagtgaacccaactgctcagtcttctggatctctatatggttcacaaatatatgccctctgcaaccaattttatacacctgcagctactggactctacgtggatcagtatttgtatcactactgtgttgtggatccccaggaggccattgccattgtactggggttcatgattattgtggcttttgctttaataattttctttgctgtgaaaactcgaagaaagatggacaggtatgacaagtccaatattttgtgggacaaggaacacatttatgatgagcagccccccaatgtcgaggagtgggttaaaaatgtgtctgcaggcacacaggacgtgccttcacccccatctgactatgtggaaagagttgacagtcccatggcatactcttccaatggcaaagtgaatgacaagcggttttatccagagtcttcctataaatccacgccggttcctgaagtggttcaggagcttccattaacttcgcctgtggatgacttcaggcagcctcgttacagcagcggtggtaactttgagacaccttcaaaaagagcacctgcaaagggaagagcaggaaggtcaaagagaacagagcaagatcactatgagacagactacacaactggcggcgagtcctgtgatgagctggaggaggactggatcagggaatatccacctatcacttcagatcaacaaagacaactgtacaagaggaattttgacactggcctacaggaatacaagagcttacaatcagaacttgatgagatcaataaagaactctcccgtttggataaagaattggatgactatagagaagaaagtgaagagtacatggctgctgctgatgaatacaatagactgaagcaagtgaagggatctgcagattacaaaagtaagaagaatcattgcaagcagttaaagagcaaattgtcacacatcaagaagatggttggagactatgatagacagaaaacatagaaggctgatgccaagttgtttgagaaattaagtatctgacatctctgcaatcttctcagaaggcaaatgactttggaccataaccccggaagccaaacctctgtgagcatcacaaagttttggttgctttaacatcatcagtattgaagcattttataaatcgcttttgataatcaactgggctgaacactccaattaaggattttatgctttaaacattggttcttgtattaagaatgaaatactgtttgaggtttttaagccttaaaggaaggttctggtgtgaactaaactttcacaccccagacgatgtcttcatacctacatgtatttgtttgcataggtgatctcatttaatcctctcaaccacctttcagataactgttatttataatcacttttttccacataaggaaactgggttcctgcaatgaagtctctgaagtgaaactgcttgtttcctagcacacacttttggttaagtctgttttatgacttcattaataataaattccctggcctttcatattttagctactatatatgtgatgatctaccagcctccctattttttttctgttatataaatggttaaaagaggtttttcttaaataataaagatcatgtaaaagtaaaaaaaaaa

SEQ ID NO: 9 - protein, 230 aa, Homo sapiens
MASLGLQLVGYILGLLGLLGTLVAMLLPSWKTSSYVGASIVTAVGFSKGLWMECATHSTGITQCDIYSTLLGLPADIQAAQAMMVTSSAISSLACIISVVGMRCTVFCQESRAKDRVAVAGGVFFILGGLLGFIPVAWNLHGILRDFYSPLVPDSMKFEIGEALYLGIISSLFSLIAGIILCFSCSSQRNRSNYYDAYQAQPLATRSSPRPGQPPKVKSEFNSYSLTGYV

SEQ ID NO: 10 - DNA, 2961 bp, Homo sapiens
gcagatggattttgcaaagctgtggttaacgattagaaatcctttatcacctcagcccgtggccccttgtacttcgctcccctccctcaggatccctttctccctctccaggggcatctccccctccaaggctctgcaaagaactgccctgtcttctagatgccttcttgaggctgcttgtggccacccacagacacttgtaaggaggagagaagtcagcctggcagagagactctgaaatgagggattagaggtgttcaaggagcaagagcttcagcctgaagacaagggagcagtccctgaagacgcttctactgagaggtctgccatggcctctcttggcctccaacttgtgggctacatcctaggccttctggggcttttgggcacactggttgccatgctgctccccagctggaaaacaagttcttatgtcggtgccagcattgtgacagcagttggcttctccaagggcctctggatggaatgtgccacacacagcacaggcatcacccagtgtgacatctatagcacccttctgggcctgcccgctgacatccaggctgcccaggccatgatggtgacatccagtgcaatctcctccctggcctgcattatctctgtggtgggcatgagatgcacagtcttctgccaggaatcccgagccaaagacagagtggcggtagcaggtggagtctttttcatccttggaggcctcctgggattcattcctgttgcctggaatcttcatgggatcctacgggacttctactcaccactggtgcctgacagcatgaaatttgagattggagaggctctttacttgggcattatttcttccctgttctccctgatagctggaatcatcctctgcttttcctgctcatcccagagaaatcgctccaactactacgatgcctaccaagcccaacctcttgccacaaggagctctccaaggcctggtcaacctcccaaagtcaagagtgagttcaattcctacagcctgacagggtatgtgtgaagaaccaggggccagagctggggggtggctgggtctgtgaaaaacagtggacagcaccccgagggccacaggtgagggacactaccactggatcgtgtcagaaggtgctgctgaggatagactgactttggccattggattgagcaaaggcagaaatgggggctagtgtaacagcatgcaggttgaattgccaaggatgctcgccatgccagcctttctgttttcctcaccttgctgctcccctgccctaagtccccaaccctcaacttgaaaccccattcccttaagccaggactcagaggatccctttgccctctggtttacctgggactccatccccaaacccactaatcacatcccactgactgaccctctgtgatcaaagaccctctctctggctgaggttggctcttagctcattgctggggatgggaaggagaagcagtggcttttgtgggcattgctctaacctacttctcaagcttccctccaaagaaactgattggccctggaacctccatcccactcttgttatgactccacagtgtccagactaatttgtgcatgaactgaaataaaaccatcctacggtatccagggaacagaaagcaggatgcaggatgggaggacaggaaggcagcctgggacatttaaaaaaataaaaatgaaaaaaaaacccagaacccatttctcagggcactttccagaattctctcatatttgtgggctgggatcaagcctgcagcttgaggaaagcacaaggaaaggaaagaagatctggtggaaagctcaggtggcagcggactctgactccactgaggaactgcctcagaagctgcgatcacaactttggctgaagcccctgcctcactctagggcacctgacctggcctcttgcctaaaccacaaggctaagggctatagacaatggtttccttaggaacagtaaaccagtttttctagggatggcccttggctgggggatgacagtgtgggagctgtggggtactgaggaagacaccattccttgacggtgtctaagaagccaggtggatgtgtgtggtggctccagtgggtgtttctactctgccagtgagaggcagccccctagaaactcttcaggcgtaatggaaaatcagctcaaatgagatcaggcccccccagggtccacccacagagcactacagagcctctgaaagaccatagcaccaagcgagccccttcagattcccccactgtccatcggaagatgctccagagtggctagagggcatctaagggctccagcatggcatatccatgcccacggtgctgtgtccatgatctgagtgatagctgcactgctgcctgggattgcagctgaggtgggagtggagaatggttcccaggaagacagttccacctctaaggtccgaaaatgttccctttaccctggagtgggagtgaggggtcatacaccaaaggtattttccctcaccagtctaggcatgactggcttctgaaaaattccagcacacctcctcgaacctcattgtcagcagagagggcccatctgttgtctgtaacatgcctttcacatgtccaccttcttgccatgttccagctgctctcccaacctggaaggccgtctccccttagccaagtcctcctcaggcttggagaacttcctcagcgtcacctccttcattgagccttctctgatcactccatccctctcctacccctccctcccccaaccctcaatgtataaattgcttcttgatgcttagcattcacaatttttgattgatcgttatttgtgtgtgtgtgtccgatctcacaagtatattgtaaacccttcggtgggtgggggccatatcctagacctctctgtatcccccagactatctgtaacagtgccaggcacacagtaggtgatcaataaacacttgttgattgag

SEQ ID NO: 11 - protein, 218 aa, Homo sapiens
MGSAALEILGLVLCLVGWGGLILACGLPMWQVTAFLDHNIVTAQTTWKGLWMSCVVQSTGHMQCKVYDSVLALSTEVQAARALTVSAVLLAFVALFVTLAGAQCTTCVAPGPAKARVALTGGVLYLFCGLLALVPLCWFANIVVREFYDPSVPVSQKYELGAALYIGWAATALLMVGGCLLCCGAWVCTGRPDLSFPVKYSAPRRPTATGDYDKKNYV

SEQ ID NO: 12 - DNA, 1384 bp, Homo sapiens
ggcagacccaggaggtgcgacagacccgcggggcaaacggactggggccaagagccgggagcgcgggcgcaaaggcaccagggcccgcccagggcgccgcgcagcacggccttgggggttctgcgggccttcgggtgcgcgtctcgcctctagccatggggtccgcagcgttggagatcctgggcctggtgctgtgcctggtgggctgggggggtctgatcctggcgtgcgggctgcccatgtggcaggtgaccgccttcctggaccacaacatcgtgacggcgcagaccacctggaaggggctgtggatgtcgtgcgtggtgcagagcaccgggcacatgcagtgcaaagtgtacgactcggtgctggctctgagcaccgaggtgcaggcggcgcgggcgctcaccgtgagcgccgtgctgctggcgttcgttgcgctcttcgtgaccctggcgggcgcgcagtgcaccacctgcgtggccccgggcccggccaaggcgcgtgtggccctcacgggaggcgtgctctacctgttttgcgggctgctggcgctcgtgccactctgctggttcgccaacattgtcgtccgcgagttttacgacccgtctgtgcccgtgtcgcagaagtacgagctgggcgcagcgctgtacatcggctgggcggccaccgcgctgctcatggtaggcggctgcctcttgtgctgcggcgcctgggtctgcaccggccgtcccgacctcagcttccccgtgaagtactcagcgccgcggcggcccacggccaccggcgactacgacaagaagaactacgtctgagggcgctgggcacggccgggcccctcctgccagccacgcctgcgaggcgttggataagcctggggagccccgcatggaccgcggcttccgccgggtagcgcggcgcgcaggctcctcggaacgtccggctctgcgccccgacgcggctcctggatccgctcctgcctgcgcccgcagctgaccttctcctgccactagcccggccctgcccttaacagacggaatgaagtttccttttctgtgcgcggcgctgtttccataggcagagcgggtgtcagactgaggatttcgcttcccctccaagacgctgggggtcttggctgctgccttacttcccagaggctcctgctgacttcggaggggcggatgcagagcccagggcccccaccggaagatgtgtacagctggtctttactccatcggcagggcccgagcccagggaccagtgacttggcctggacctcccggtctcactccagcatctccccaggcaaggcttgtgggcaccggagcttgagagagggcgggagtgggaaggctaagaatctgcttagtaaatggtttgaactctc

SEQ ID NO: 13 - protein, 220 aa, Homo sapiens
MASAGMQILGVVLTLLGWVNGLVSCALPMWKVTAFIGNSIVVAQVVWEGLWMSCVVQSTGQMQCKVYDSLLALPQDLQAARALCVIALLVALFGLLVYLAGAKCTTCVEEKDSKARLVLTSGIVFVISGVLTLIPVCWTAHAIIRDFYNPLVAEAQKRELGASLYLGWAASGLLLLGGGLLCCTCPSGGSQGPSHYMARYSTSAPAISRGPSEYPTKNYV

SEQ ID NO: 14 - DNA, 1366 bp, Homo sapiens
actcggcctaggaatttcccttatctccttcgcagtgcagctccttcaacctcgccatggcctctgccggaatgcagatcctgggagtcgtcctgacactgctgggctgggtgaatggcctggtctcctgtgccctgcccatgtggaaggtgaccgctttcatcggcaacagcatcgtggtggcccaggtggtgtgggagggcctgtggatgtcctgcgtggtgcagagcaccggccagatgcagtgcaaggtgtacgactcactgctggcgctgccacaggacctgcaggctgcacgtgccctctgtgtcatcgccctccttgtggccctgttcggcttgctggtctaccttgctggggccaagtgtaccacctgtgtggaggagaaggattccaaggcccgcctggtgctcacctctgggattgtctttgtcatctcaggggtcctgacgctaatccccgtgtgctggacggcgcatgccatcatccgggacttctataaccccctggtggctgaggcccaaaagcgggagctgggggcctccctctacttgggctgggcggcctcaggccttttgttgctgggtggggggttgctgtgctgcacttgcccctcgggggggtcccagggccccagccattacatggcccgctactcaacatctgcccctgccatctctcgggggccctctgagtaccctaccaagaattacgtctgacgtggaggggaatgggggctccgctggcgctagagccatccagaagtggcagtgcccaacagctttgggatgggttcgtaccttttgtttctgcctcctgctatttttcttttgactgaggatatttaaaattcatttgaaaactgagccaaggtgttgactcagactctcacttaggctctgctgtttctcacccttggatgatggagccaaagaggggatgctttgagattctggatcttgacatgcccatcttagaagccagtcaagctatggaactaatgcggaggctgcttgctgtgctggctttgcaacaagacagactgtccccaagagttcctgctgctgctgggggctgggcttccctagatgtcactggacagctgccccccatcctactcaggtctctggagctcctctcttcacccctggaaaaacaaatgatctgttaacaaaggactgcccacctccggaacttctgacctctgtttcctccgtcctgataagacgtccaccccccagggccaggtcccagctatgtagacccccgcccccacctccaacactgcacccttctgccctgcccccctcgtctcaccccctttacactcacatttttatcaaataaagcatgttttgttagtgca

SEQ ID NO: 15 - protein, 211 aa, Homo sapiens
MANSGLQLLGFSMALLGWVGLVACTAIPQWQMSSYAGDNIITAQAMYKGLWMDCVTQSTGMMSCKMYDSVLALSAALQATRALMVVSLVLGFLAMFVATMGMKCTRCGGDDKVKKARIAMGGGIIFIVAGLAALVACSWYGHQIVTDFYNPLIPTNIKYEFGPAIFIGWAGSALVILGGALLSCSCPGNESKAGYRVPRSYPKSNSSKEYV

SEQ ID NO: 16 - DNA, 1542 bp, Homo sapiens
gcccgcacctgctggctcacctccgagccacctctgctgcgcaccgcagcctcggacctacagcccaggatactttgggacttgccggcgctcagaaacgcgcccagacggcccctccaccttttgtttgcctagggtcgccgagagcgcccggagggaaccgcctggccttcggggaccaccaattttgtctggaaccaccctcccggcgtatcctactccctgtgccgcgaggccatcgcttcactggaggggtcgatttgtgtgtagtttggtgacaagatttgcattcacctggcccaaaccctttttgtctctttgggtgaccggaaaactccacctcaagttttcttttgtggggctgccccccaagtgtcgtttgttttactgtagggtctccccgcccggcgcccccagtgttttctgagggcggaaatggccaattcgggcctgcagttgctgggcttctccatggccctgctgggctgggtgggtctggtggcctgcaccgccatcccgcagtggcagatgagctcctatgcgggtgacaacatcatcacggcccaggccatgtacaaggggctgtggatggactgcgtcacgcagagcacggggatgatgagctgcaaaatgtacgactcggtgctcgccctgtccgcggccttgcaggccactcgagccctaatggtggtctccctggtgctgggcttcctggccatgtttgtggccacgatgggcatgaagtgcacgcgctgtgggggagacgacaaagtgaagaaggcccgtatagccatgggtggaggcataattttcatcgtggcaggtcttgccgccttggtagcttgctcctggtatggccatcagattgtcacagacttttataaccctttgatccctaccaacattaagtatgagtttggccctgccatctttattggctgggcagggtctgccctagtcatcctgggaggtgcactgctctcctgttcctgtcctgggaatgagagcaaggctgggtaccgtgtaccccgctcttaccctaagtccaactcttccaaggagtatgtgtgacctgggatctccttgccccagcctgacaggctatgggagtgtctagatgcctgaaagggcctggggctgagctcagcctgtgggcagggtgccggacaaaggcctcctggtcactctgtccctgcactccatgtatagtcctcttgggttgggggtgggggggtgccgttggtgggagagacaaaaagagggagagtgtgctttttgtacagtaataaaaaataagtattgggaagcaggcttttttcccttcagggcctctgctttcctcccgtccagatccttgcagggagcttggaaccttagtgcacctacttcagttcagaacacttagcaccccactgactccactgacaattgactaaaagatgcaggtgctcgtatctcgacattcattcccacccccctcttatttaaatagctaccaaagtacttcttttttaataaaaaaataaagatttttattaggta

SEQ ID NO: 17 - DNA, 20 bp, Artificial Sequence
  A synthetic sequence
ccggttcccgggaagttacg

SEQ ID NO: 18 - DNA, 20 bp, Artificial Sequence
  A synthetic sequence
cagggggagggaattcaact

SEQ ID NO: 19 - DNA, 20 bp, Artificial Sequence
  A synthetic sequence
ctttcgcagcccggccacgt

SEQ ID NO: 20 - DNA, 20 bp, Artificial Sequence
  A synthetic sequence
gggaagttacgtggcgaagc

SEQ ID NO: 21 - DNA, 22 bp, Artificial Sequence
  A synthetic sequence
atgcattcaaactgaggtgcct

SEQ ID NO: 22 - DNA, 22 bp, Artificial Sequence
  A synthetic sequence
aacttcaccttccctccaacca

SEQ ID NO: 23 - DNA, 24 bp, Artificial Sequence
  A synthetic sequence
cccaaaggcaaacaacccacttct

SEQ ID NO: 24 - DNA, 24 bp, Artificial Sequence
  A synthetic sequence
agctgggtggaagagaacacagtt

SEQ ID NO: 25 - DNA, 22 bp, Artificial Sequence
  A synthetic sequence
tgttactcggcggagttcgtac

SEQ ID NO: 26 - DNA, 23 bp, Artificial Sequence
  A synthetic sequence
gatcccatccattagacacgcag

SEQ ID NO: 27 - DNA, 20 bp, Artificial Sequence
  A synthetic sequence
aaccagcgcatggacagtta

SEQ ID NO: 28 - DNA, 20 bp, Artificial Sequence
  A synthetic sequence
cgagctggtcatggagttgt

SEQ ID NO: 29 - DNA, 22 bp, Artificial Sequence
  A synthetic sequence
ccttgtgtggtatggagactgc

SEQ ID NO: 30 - DNA, 23 bp, Artificial Sequence
  A synthetic sequence
ctttcacatctgtagccttctgc

SEQ ID NO: 31 - DNA, 22 bp, Artificial Sequence
  A synthetic sequence
ggaagcactgtttgccaagacc

SEQ ID NO: 32 - DNA, 22 bp, Artificial Sequence
  A synthetic sequence
ctgttgttggcggcacttagct

SEQ ID NO: 33 - DNA, 22 bp, Artificial Sequence
  A synthetic sequence
gctgaaggacagcgagaagatc

SEQ ID NO: 34 - DNA, 23 bp, Artificial Sequence
  A synthetic sequence
gggtccattttgggctttttccg

SEQ ID NO: 35 - DNA, 24 bp, Artificial Sequence
  A synthetic sequence
ctctagtgatccctgagaagttcc

SEQ ID NO: 36 - DNA, 20 bp, Artificial Sequence
  A synthetic sequence
actcgctccacctcatcctc

SEQ ID NO: 37 - DNA, 24 bp, Artificial Sequence
  A synthetic sequence
gcagctagccagtgtacagtatac

SEQ ID NO: 38 - DNA, 24 bp, Artificial Sequence
  A synthetic sequence
gcctcagaaatccagcttctcgaa

SEQ ID NO: 39 - DNA, 20 bp, Artificial Sequence
  A synthetic sequence
tttccagatggtgagagccg

SEQ ID NO: 40 - DNA, 20 bp, Artificial Sequence
  A synthetic sequence
ccgatgcctcaactctccag

SEQ ID NO: 41 - DNA, 21 bp, Artificial Sequence
  A synthetic sequence
cccgaaactcggcaggcaaga

SEQ ID NO: 42 - DNA, 21 bp, Artificial Sequence
  A synthetic sequence
aaggctcagacttcccggcac

SEQ ID NO: 43 - DNA, 25 bp, Artificial Sequence
  A synthetic sequence
cggggagaatgtggactgggtagag

SEQ ID NO: 44 - DNA, 23 bp, Artificial Sequence
  A synthetic sequence
ctggagttacacttgggggcagc

SEQ ID NO: 45 - DNA, 21 bp, Artificial Sequence
  A synthetic sequence
gagccaagggcgagtcccgta

SEQ ID NO: 46 - DNA, 22 bp, Artificial Sequence
  A synthetic sequence
ccttccacgacttgcccagcat

SEQ ID NO: 47 - DNA, 4 bp, Artificial Sequence
  A synthetic sequence
ngcg

SEQ ID NO: 48 - DNA, 4 bp, Artificial Sequence
  A synthetic sequence
ngag

SEQ ID NO: 49 - DNA, 4 bp, Artificial Sequence
  A synthetic sequence
ngan

SEQ ID NO: 50 - DNA, 4 bp, Artificial Sequence
  A synthetic sequence
ngng

SEQ ID NO: 51 - DNA, 6 bp, Artificial Sequence
  A synthetic sequence
nngnnt

SEQ ID NO: 52 - DNA, 6 bp, Artificial Sequence
  A synthetic sequence
nnnnnt

(19) United States
(12) Patent Application Publication
(10) Pub. No.: US 2025/0263654 A1
(43) Pub. Date: Aug. 21, 2025

(54) PRIMORDIAL GERM CELLS

(71) Applicants: The J. David Gladstone Institutes, a testamentary trust established under the will of J. David, San Francisco, CA (US); The Regents of the University of California, Oakland, CA (US)

(72) Inventors: Todd C. McDevitt, San Francisco, CA (US); Ivana Vasic, Oakland, CA (US)

(21) Appl. No.: 18/569,550

(22) PCT Filed: Jun. 24, 2022
(86) PCT No.: PCT/US2022/034869

§ 371 Date: Dec. 12, 2023

Related U.S. Application Data
(60) Provisional application No. 63/214,901, filed on Jun. 25, 2021.

(51) Int. Cl. C12N 5/0735; C12N 9/22; C12N 15/11; C12N 15/113

(52) U.S. Cl. CPC C12N 5/0611; C12N 9/226; C12N 15/111; C12N 15/113; C12N 2310/20; C12N 2501/155; C12N 2501/727; C12N 2506/45; C12N 2510/00; C12N 2533/90

(57) ABSTRACT

Described herein are compositions, systems, and methods for obtaining primordial germ cells (PGCs) from pluripotent stem cells (PSCs). Inhibiting or bypassing tight junction formation in a population of pluripotent stem cells to generate a modified cell population, and contacting the modified cell population with BMP. Where inhibiting or bypassing tight junction formation includes incubating the population of pluripotent stem cells.

### CROSS REFERENCE TO RELATED APPLICATIONS

This application is a U.S. National Stage Filing under 35 U.S.C. 371 from International Patent Application Serial No. PCT/US2022/034869, filed Jun. 24, 2022, Published as WO2022/272042 on Dec. 29, 2022, which application claims the benefit of priority to U.S. Provisional Patent Application Ser. No. 63/214,901 entitled “Human Primordial Germ Cells from Human Induced Pluripotent Stem Cells,” filed Jun. 25, 2021, the complete disclosures of which are incorporated herein by reference in their entireties.

### GOVERNMENT SUPPORT

This invention was made with government support under CBET 0939511 awarded by the National Science Foundation. The government has certain rights in the invention.

### INCORPORATION BY REFERENCE OF SEQUENCE LISTING

This application contains a Sequence Listing which has been submitted electronically in ST25 format and hereby incorporated by reference in its entirety. Said ST256 file, created on Jul. 3, 2024, is name 3730194US1.txt and is 80,536 bytes in size.

### BACKGROUND

Human primordial germ cells (PGCs) are the precursors to human male and female sex cells (spermatozoa and oocytes). Ethical considerations largely prevent close interrogation of the development and specification of primordial germ cells in a human embryo. If primordial germ cells could be generated in vitro they could be used to differentiate functional oocytes and spermatozoa that could be used for In Vitro Fertilization (IVF), which would address a range of problems that currently plague IVF treatments such as: low retrieval of oocytes, ovarian hyperstimulation syndrome (which occurs during the hormone treatments to retrieve the oocytes), and senescence of sex cell production for older couples.

Embryonic pluripotent stem cells (PSCs) are taken directly from the inner cell mass/epiblast of a human embryo. Induced PSCs are reprogrammed from somatic cells taken from a patient (through methods such as a skin biopsy, blood draw, cheek swab, etc.). Typically, when these embryonic or induced PSCs are cultured in vitro, they form a polarized epithelial “barrier” structure and are considered “primed.” Primed PSCs structurally, transcriptionally, and epigenetically resemble post-implantation/pre-gastrulation (E9-E12) pluripotent stem cells in the epiblast and have the potential to form any somatic cell type (lungs, heart, kidney, skin, etc.) found in the body, if they are exposed to the correct differentiation cues.

However, researchers generally believe that cultured primed PSCs do not have the ability to form primordial germ cells (PGCs), which are the precursors to sperm and ova, because primed PSCs are thought to be too committed at this stage to a somatic developmental trajectory. Hence, currently available methods for generating primordial germ cells (PGCs) typically involve chemical treatments and/or genetic modifications to revert the primed PSCs to a more naïve state, followed by use of a several factors to induce differentiation into primordial germ cells (PGCs).

### SUMMARY

Described herein are systems, compositions, and methods for obtaining primordial germ cells (PGCs) from pluripotent stem cells (PSCs). For example, the pluripotent stem cells employed can be human induced pluripotent stem cells (hiPSCs)). The PSCs can be genetically modified (e.g., to repair genetic mutations or to facilitate PGC differentiation). In some cases, the PSCs can be genetically modified to express genes involved in PGC specification or genetically modified to make the PSCs more susceptible to PGC differentiation.

However, as described herein, such genetic modification is not needed to produce primordial germ cells from PSCs. Instead, an effective method is described herein that involves basolateral stimulation of human induced pluripotent stem cells with BMP. For example, the methods can involve seeding PSCs into vessels that provide BMP with basolateral access to the PSCs.

PGCs are the first step to differentiating functional oocytes and spermatozoa that can be used for In Vitro Fertilization (IVF). The methods described herein allow men and women who are experiencing fertility problems to undergo a simple cell retrieval (e.g., a simple skin biopsy), followed by reprogramming of their cells into hiPSCs and differentiation of the hiPSCs into PGCs. The PGCs can then be differentiated into functional sex cells. Use of such iPSC-derived PGCs addresses a range of problems that currently plague IVF treatments, such as: low retrieval of oocytes, ovarian hyperstimulation syndrome (which occurs during the hormone treatments to retrieve the oocytes), and senescence of sex cell production for older couples. Additionally, simple and non-invasive PGC derivation facilitates screening of genetic disease for at-risk couples, enabling trans-differentiation and IVF of sex cells for same sex couples. Some beneficial products and methods provided are:
1. Minimally invasive and hormone-free oocyte retrieval:
   a. No physician monitoring is needed,
   b. No expensive hospital visits/hormone treatments are needed,
   c. Cheaper and more efficient derivation of PGCs and oocytes.
2. Derivation of oocytes and spermatozoa from older patients with traditionally less sex cell production viability.
3. Expanded and biopsy-free screening for genetic disease.
4. Trans-differentiation of PGCs to oocytes/spermatozoa of opposite sex.

Methods and systems are described herein that are useful for generating primordial germ cells. Such methods can involve reducing or bypassing barrier function in a population of pluripotent stem cells to generate modified cell population and contacting the modified cell population with BMP. For example, the methods can involve inhibiting or bypassing tight junction formation in a population of pluripotent stem cells to generate a modified cell population, and contacting the modified cell population with BMP. As used herein, “inhibiting tight junction(s)” means reducing the incidence of tight junction formation, maintaining pluripotent stem cells in a naïve state, and/or bypassing tight junction formation. Inhibiting or bypassing tight junction formation can include:
a. incubating the population of pluripotent stem cells on a porous surface to bypass apical tight junctions;
b. contacting the population of pluripotent stem cells with one or more inhibitory nucleic acids that bind one or more tight junction nucleic acids (one or more tight junction mRNA or DNA);
c. contacting the population of pluripotent stem cells with one or more CRISPRi ribonucleoprotein (RNP) complexes targeted to one or more tight junction gene;
d. contacting the population of pluripotent stem cells with one or more expression vectors or virus-like particles (VLP) encoding one or more guide RNAs that can bind one or more tight junction gene;
e. contacting the population of pluripotent stem cells with one or more chelators (e.g., calcium chelators) or chemical inhibitors; or
f. combinations thereof.
   The modified cell population is modified relative to a control cell population that has not be treated or manipulated to inhibit or bypass tight junction formation.

In some cases pluripotent stem cells can be supported on a porous surface in a culture medium that contains BMP. This method does not require genetic modification of the pluripotent stem cells to provide primordial germ cells. The porous surface can be a membrane that freely allows nutrients and morphogens (e.g., proteins such as BMP) to circulate through the membrane. One type of culture apparatus that includes a porous surface for culture of the cells is a transwell culture system. Examples of materials that can be used for the porous surface include porous polycarbonate, polyester (PET), and/or collagen-coated polytetrafluoroethylene (PTFE) materials.

The pluripotent stem cells can be induced pluripotent stem cells (iPSCs), such as human induced pluripotent stem cells (hiPSCs). Cells can be obtained from a selected subject, iPSCs can be generated from the subject's cells, and those iPSCs can then be converted into primordial germ cells. Mature germ cells can be generated from the primordial germ cells and used for in vitro fertilization to provide an embryo that can be implanted for gestation in a female. Hence, the pluripotent stem cells or the induced pluripotent stem cells can be autologous or allogenic to a subject who desires in vitro fertilization. The subject can be any mammalian or avian subject. In addition to human subjects, the methods and systems can be used to provide primordial germ cells for domesticated animals, wild animal species, endangered animal species (e.g., an animal on an endangered species list), as well as animal species that are extinct or are in danger of becoming extinct.

The pluripotent stem cells can be genetically modified. For example, the pluripotent stem cells can be genetically modified to correct a genetic defect.

In some cases the pluripotent stem cells can be genetically modified to reduce the expression or function of an endogenous tight junction gene. For example, such a tight junction gene can be at least one endogenous zonula occludens-1 (ZO1), zonula occludens-2 (ZO2), zonula occludens-3 (ZO3), OCLN, CLDN2, CLDN5, CLDN6, or CLDN7 gene. At least one tight junction allele of any of these genes can be genetically modified. In some cases, two tight junction alleles of any of these genes can be genetically modified.

The BMP used in the system can be BMP2, BMP4, or a combination thereof.

Also described herein are methods that involve incubating one or more pluripotent stem cells on a porous surface within a system comprising in a culture medium that contains BMP. The pluripotent stem cells can be induced pluripotent stem cells (iPSCs), such as human induced pluripotent stem cells (hiPSCs). The pluripotent stem cells can be genetically modified. For example, the pluripotent stem cells can be genetically modified to correct a genetic defect.

The methods and systems described herein can involve culturing cells on porous surfaces (e.g., a transwell) under conditions that provide growth of the cells. Such a porous surface (e.g., transwell) can have an apical compartment as well as a basolateral compartment. The pluripotent stem cells can be one the porous surface in the apical compartment and receive BMP from at least a basolateral compartment.

The conditions used for generating PGCs can include culturing the cells at temperatures above 30° C., or above 33° C., or above 35° C., or above 36° C. The temperature should be below 42° C., or below 40° C., or below 39° C., or below 38° C. For example, the temperature can be about 37° C. The culture medium can include a ROCK inhibitor.

### DESCRIPTION OF THE FIGURES

FIG. 1A-1H illustrate knockdown of zonula occludens-1 (ZO1) in human induced pluripotent stem cells (hiPSCs) and the functional consequences of such knockdown. FIG. 1A is a schematic illustrating the CRISPR-interference platform used to knockdown zonula occludens-1 (ZO1) in hiPSCs. Briefly, a TET-responsive dead Cas9-KRAB construct was knocked into the AAVS1 locus of the hiPSCs. dCas9-KRAB was expressed upon addition of Doxycycline (DOX). Upon constitutive expression of a ZO1 guide RNA (designed by the inventors), transcription of ZO1 was blocked. FIG. 1B shows expression of ZO1 and the nuclear marker Lamin-B1 (LMNB1) in the hiPSCs after exposure of the cells to Doxycycline (2 uM) for several days to induce knockdown of ZO1. As illustrated, by day 5, ZO1 expression was not visibly detectable in these ZO1 knockdown cells. FIG. 1C graphically illustrates the fold change of ZO1 expression after exposure of the hiPSCs to Doxycycline (2 uM) for five days to induce knockdown of ZO1. As illustrated, by day 5, ZO1 expression was substantially undetectable. FIG. 1D illustrates fluorescent measurements of media aliquots taken over time from the basolateral side of a transwell in which a wild type cell layer or a ZO1 knockdown cell layer was maintained after addition of FITC-dextran to the apical side of the transwell. As illustrated, the wild type cell layer forms a membrane that is less permeable to the FITC-dextran than is the ZO1 knockdown cell layer. This graph illustrates how barrier function and ability to preclude diffusion of molecules from one side of a cellular monolayer to the other (apical to basolateral diffusion) is disrupted by ZO1 knockdown. FIG. 1E graphically illustrates transepithelial resistance in wild type and ZO1-knockdown cells treated for 5 days with Doxycycline (2 uM), indicating loss of barrier function with ZO1 knockdown. FIG. 1F shows images of wild type and ZO1 knockdown cells immunostained for the nuclear marker Lamin-B1 (LMNB1) or for cytovillin (EZRIN), an apical polarity protein. As shown, expression of EZRIN is attenuated with ZO1 knockdown cells, indicating loss of apical/basolateral polarity. FIG. 1G shows chromosomal images illustrating the karyotype of a ZO1 WTC-LMNB1-GFP-CRISPRi (male ZO1 knockdown line). FIG. 1H illustrates karyotyping analysis of expression from chromosomal loci demonstrating that all genetically modified lines used to validate results in this study are karyotypically normal, including the ZO1 WTC-LMNB1-GFP-CRISPRi (male ZO1 knockdown line), ZO1 WTB-CRISPRi-Gen1B (female ZO1 knockdown line) and ZO1 WTC-NANOS3-mCHERRY (male ZO1 knockdown line, with PGC reporter).

FIG. 2A-2E illustrate the method by which PGCLCs (primordial germ like cells, designated “like” because they are generated in vitro) are generated from ZO1 wild type and ZO1 knockdown hiPSCs. FIG. 2A illustrates that as a result of impaired barrier function, ZO1 knockdown hiPSCs lose polarized response to BMP4, enabling activation of pSMAD1 when BMP4 is presented apically (apical presentation is typical in standard/non-transwell culture). FIG. 2B is a schematic illustrating methods for determining specification bias, which was used to assay the ZO1 knockdown cells in comparison to ZO1 wild type cells. FIG. 2C shows the results of the specification bias assay delineated in FIG. 2B, demonstrating that ZO1 knockdown cells have marked bias for expressing PGC markers (BLIMP1), but also expressed SOX17, CDX2, T-box transcription factor T (TBXT or T), and SOX2. Wild type cells exhibited more SOX2 expression while ZO1 knockdown cells exhibited more BLIMP1 and TBXT expression. FIG. 2D graphically illustrates qPCR data from monolayers of control cells (−DOX) and ZO1 knockdown cells (+5 days of DOX or +14 days of DOX), treated with BMP4 for 48 hours. These results demonstrate that BMP4-treated ZO1 knockdown cells exhibit significant increases in PGC transcription factors (T, SOX17, NANOS3, and BLIMP1), validating immunofluorescent staining data from FIG. 2C. FIG. 2E shows replicate immunofluorescent staining of control (−DOX) and ZO1 knockdown (+DOX) cells after treatment with BMP4 for 48 hours to detect a panel of PGC markers (BLIMP1, SOX17, and TFAP2C). Double positive staining was used to identify primordial germ cell like cells (PGCLCs; which are primordial germ cells generated in vitro). SOX2 is not a PGC marker and was shown as a negative control. In the original SOX2 was stained blue, TFAP2C was stained blue, BLIMP1 was stained red, and SOX17 was stained green.

FIG. 3 schematically illustrates that PGC (also called PGCLC) differentiation can be achieved via ZO1 silencing, pharmacological inhibition of ZO1, or by growth of cells on transwell membranes in the presence of BMP4. Such growth of cells on transwell membranes requires no chemical and no structural perturbation cells, and instead is mediated by basolateral stimulation by BMP. These varied methods illustrate that loss of barrier function or heightened accessibility of BMP4 to its basolateral receptors leads to high activation of the canonical BMP-SMAD1 pathway (illustrated in FIG. 2A). For comparison, a typical epithelial cell layer in culture is schematically illustrated on the left, which forms tight junctions maintained by ZO1 and which does not produce PGCs (PGCLCs) upon stimulation with BMP4.

FIG. 4 schematically illustrates the role of Zonula occludens-1 (ZO1, also called TJP1) within cells and how ZO1 maintains epithelial structure. ZO1 is a tight junction protein expressed in primed pluripotent stem cells in standard in vitro culture. ZO1 forms dual-purpose adhesion plaques that endow an epithelium with both barrier and partitioning functions (polarity/directionality), thereby attenuating responses to morphogen signals (such as BMP4).

FIG. 5A-5H illustrate that unconfined human iPSC colonies undergo radial gastrulation-like patterning with loss of ZO1 on the colony edge. FIG. 5A illustrates a method where hiPSCs were aggregated into pyramidal wells, subsequently plated, and induced with BMP4 for 48 hours. FIG. 5B illustrates that unconfined colonies of wild type hiPSCs undergo radial patterning of gastrulation-associated markers after 48 hrs of BMP4 stimulation. FIG. 5C shows immunofluorescence images of a wild type colony edge, showing loss of ZO1 and gain of pSMAD1 at the colony edge. FIG. 5D graphically illustrates quantification of ZO1 loss and pSMAD1 gain on wild type colony edges (n=3). FIG. 5E shows images of unconfined and low/high density micropatterned colonies, with a comparison of ZO1 and pSMAD1 expression in these wild type colonies. FIG. 5F shows images of unconfined wild type colonies illustrating that they maintain honeycomb ZO1 expression over time. FIG. 5G graphically illustrates cell density measurements in unconfined wild type colonies, with a projected density curve for micropatterned colonies (assuming density of 5,000 cells/mm² upon induction with BMP4). Epithelial range, based on structure of cell-cell junction pattern, was estimated to be in the range of 3,000-10,000 cells/mm². FIG. 5H shows images of wild type cellular monolayers illustrating ZO1 and pSMAD1 expression as a function of cell density in monolayer culture. The epithelial structure (honeycomb cell-cell junction pattern) is lost and pSMAD1 activation is increased as cell density increases.

FIG. 6A-6I illustrate that ZO1 knockdown (ZKD) causes ubiquitous and sustained phosphorylation of SMAD1 throughout cellular colonies over time. FIG. 6A is a schematic illustrating that CRISPRi knockdown of ZO1 increases signaling protein accessibility. FIG. 6B shows a Western blot illustrating ZO1 protein loss in the ZO1 knockdown cell lines. The WTB (female) and WTC (male) cells are parental hiPSC lines. FIG. 6C shows immunofluorescence images and brightfield images illustrating morphological differences between ZO1 wild type and ZO1 knockdown cells. FIG. 6D graphically illustrates changes in nuclear height, area, cell density, and growth rate of ZO1 wild type and ZO1 knockdown cells. FIG. 6E graphically illustrates the fraction of pSMAD1+ cells over time, normalized to expression of LMNB1 (n≥3), in populations of ZO1 wild type and ZO1 knockdown cells. FIG. 6F shows immunofluorescence images illustrating maintained and ubiquitous phosphorylation of SMAD1 in ZO1 knockdown (ZKD) cells compared to ZO1 wild type cells over the course of 48 hours. FIG. 6G is a schematic illustrating a FITC-dextran diffusion assay. ZO1 wild type (ZWT) and ZO1 knockdown (ZKD) cells were cultured on a transwell plate, 40 kDa FITC was applied to the apical side, and fluorescence measurements were taken from the basolateral compartment over time. FIG. 6H graphically illustrates the fluorescence observed from the basolateral compartment over time using the method illustrated in FIG. 6G. FIG. 6I graphically illustrates transepithelial electrical resistance (TEER) measurements in ZO1 wild type (ZWT) and ZO1 knockdown (ZKD) monolayers.

FIG. 7A-7N illustrate that ZO1 knockdown (ZKD) cells are biased toward differentiation into PGCs. FIG. 7A is a schematic showing the inventors' predictions regarding spatial emergence of distinct lineages arising in ZO1 wild type (ZWT; top) and ZO1 knockdown (ZKD; bottom) colonies exposed to BMP4 under a reaction diffusion (RD)/positional information (PI) patterning model. FIG. 7B shows immunofluorescence images of canonical germ lineage markers LMNB1, CDX2, SOX2, TBXT in ZO1 wild type (ZWT) and ZO1 knockdown (ZKD) cells after 48 hours of stimulation with BMP4. FIG. 7C graphically illustrates the fraction of cells positive for expression of the markers shown in FIG. 7B in wild type (ZWT) and ZO1 knockdown (ZKD) cells. FIG. 7D shows a volcano plot of RNA sequencing data illustrating log fold changes of SOX2, TBXT, and CDX2. FIG. 7E graphically illustrates RNA sequencing data illustrating expression levels of canonical germ layer markers in ZO1 wild type (ZWT) and ZO1 knockdown (ZKD) cells after 48 hours of stimulation with BMP4. FIG. 7F illustrates unbiased clustering of the top 16 differentially expressed genes between ZO1 wild type (ZWT) and ZO1 knockdown (ZKD) cells, highlighting increases in PGC-related genes. FIG. 7G shows immunofluorescent images of LMNB1, and PGC markers BLIMP1, SOX17, TFAP2C in ZO1 wild type (ZWT) and ZO1 knockdown cells (ZKD) after 48 hours of stimulation with BMP4. FIG. 7H illustrates that pSMAD1 expression is only activated upon basolateral (top row) BMP4 stimulation in wild type ZO1 cells, but not by apical BMP4 stimulation. However, both apical and basolateral stimulation by BMP activates pSMAD1 in ZO1 knockdown (ZKD) cells. FIG. 7I graphically illustrates levels of BMP receptor expression in ZO1 wild type and ZO1 knockdown cells as observed from RNA sequencing data. The types of BMP receptors are recited along the x-axis. FIG. 7J graphically illustrates the fold change in secreted morphogens at 12 hours of BMP4 stimulation, showing significant increases in Noggin (NOG) in the ZO1 knockdown (ZKD) cells that are not seen in ZO1 wild type cells, as detected by qPCR. FIG. 7K shows images of cells illustrating the positioning of the Golgi in ZO1 wild type (left) and ZO1 knockdown (right) cells. Z-stacks revealed that in both cell types, the Golgi sits on top of the nucleus on the apical side of the cell, indicating that polarity of the ZO1 knockdown cells is still intact. FIG. 7L graphically illustrates the fluorescence intensity of immunostained Golgi as a function of the distance from the nuclear center of ZO1 wild type and ZO1 knockdown cells, indicating that the Golgi sits on top of the nucleus on the apical side of both cell types. FIG. 7M shows images of immunofluorescent-stained ZO1 wild type cells (left) and ZO1 knockdown cells (right), illustrating that ZO1 knockdown cells lost apical Ezrin expression (dark area delineated by a white dashed line). Even in regions where Ezrin is present, the Ezrin overlaps significantly with BMPR1A (a basolateral BMP receptor). FIG. 7N graphically illustrates the ratio of EZRIN: BMPR1A in ZO1 wild type and ZO1 knockdown cells. Hence, changes occur in the amounts and localization of some apical/basolateral elements in ZO1 knockdown cells compared to wild type cells.

FIG. 8A-8H illustrate ZO1 knockdown cells have a bias for PGC differentiation. FIG. 8A shows images of immunofluorescent-stained ZO1 wild type and ZO1 knockdown cells illustrating expression of LMNB1, BLIMP1, SOX17, TFAP2C after 48 hours and 72 hours of stimulation with BMP4. FIG. 8B graphically illustrates the percent of ZO1 wild type and ZO1 knockdown cellular nuclei that exhibit expression of the indicated PGC markers (n≥3). FIG. 8C illustrates expression of canonical pluripotency markers in ZO1 wild type and ZO1 knockdown cells prior to BMP4 stimulation. FIG. 8D illustrates methylation levels of ZO1 wild type versus ZO1 knockdown cells; the data were from whole genome bisulfite sequencing data. FIG. 8E shows images of immunofluorescent-stained cells illustrating expression of LMNB1, BLIMP1, SOX17, TFAP2C in ZO1 wild type and ZO1 knockdown cells after 48 hours and 72 hours of stimulation with BMP4 in a female hiPSC line. FIG. 8F graphically illustrates the percent of ZO1 wild type and ZO1 knockdown cellular nuclei that exhibit expression of PGC markers (n≥3) in a female hiPSC line. FIG. 8G illustrates unbiased clustering of top 16 differentially expressed genes between ZO1 wild type and ZO1 knockdown cells in the pluripotent condition. FIG. 8H illustrates probe methylation levels between ZO1 wild type and ZO1 knockdown cells gathered from whole genome bisulfite sequencing data, probes with significant differences in methylation are darkly shaded.

FIG. 9A-9B illustrate that ZO1 knockdown-related PGCLC bias is a product of signaling, not changes in pluripotency. FIG. 9A shows images of immunofluorescent-stained ZO1 wild type (top) and ZO1 knockdown (bottom) cells illustrating pSMAD1 expression after basolateral BMP4 stimulation for timepoints between 0-48 hrs when the cells were grown on the transwell membranes. FIG. 9B shows images of immunofluorescent-stained ZO1 wild type and ZO1 knockdown cells illustrating expression of LMNB1, BLIMP1, SOX17, TFAP2C when the cells were grown on transwell membranes with 48 hrs of bi-directional (apical and basolateral) stimulation with BMP4 at concentrations between 5-50 ng/ml.

### DETAILED DESCRIPTION

Described herein are compositions and method for obtaining primordial germ cells (PGCs) from pluripotent stem cells (PSCs), including human induced pluripotent stem cells (hiPSCs). The compositions and methods provide useful numbers of primordial germ cells (PGCs) with an efficiency of about 50-60% and without the need for three-dimensional (3D) suspension or bioreactor culturing procedures. The epithelial barrier structure of the induced pluripotent stem cells is modified by the methods described herein either during differentiation by basolateral exposure to BMP, by exposure to tight junction inhibitors, or by using CRISPR interference (CRIPSRi) to inhibit, knock down, or knockout one or more tight junction genes or tight junction proteins.

As mentioned above, researchers generally believe that cultured primed PSCs do not have the ability to form primordial germ cells (PGCs), which are the precursors to sperm and ova, because the primed PSCs are thought to be too committed at this stage in their developmental trajectory. Hence, currently available in vitro differentiation protocols for generating PGC-like cells (PGCLCs) involve a step that causes primed PSCs to be reverted to a more naïve state first. This step is followed by a priming step, and differentiation with the morphogens BMP4 or BMP2. For example, currently available reprogramming methods involve manipulating primed PSCs to a more naïve PSC state that structurally/transcriptionally/epigenetically resembles the apolar inner cell mass/pre-implantation epiblast (E5-E9). This has been done through transient delivery of transgenes via expression vectors or by introducing RNA, or through exposure of the primed PSCs to various cytokines/histone deacetylases, and other chemicals and/or biological molecules (e.g., LIF, SCF, EGF, Activin A, CHIR99021).

However, the methods described herein do not require such genetic modification or extensive exposure to multiple chemicals and biological molecules. Instead, the methods can simply involve culturing pluripotent stem cells (e.g., human induced pluripotent stem cells (hiPSCs)) in vessels that allow BMP to basolaterally contact the pluripotent stem cells for a time sufficient for the pluripotent stem cells to differentiate into primordial germ cells. Alternatively, pluripotent stem cells (e.g., human induced pluripotent stem cells (hiPSCs)) can be cultured under conditions that transiently inhibit relevant tight junction proteins, for example, by knockdown of tight junction protein expression or through pharmacological inhibition of tight junction protein functions.

As demonstrated herein, tight junctions are assembled via the protein ZO1. Such tight junctions are used by cells to split the cell into two “sides”: the apical side and the basolateral side. Apical refers to the outward-facing side(s) of a cell, which have more tight junctions than the basolateral side of cell. Basolateral refers to the inward-facing side(s) of a cell. When cells are cultured on a plate or surface, the apical side is the side exposed to culture media, while the basolateral side is the side facing/attached to the plate or surface of the culture vessel.

Tight junctions can prevent diffusion of proteins and other small molecules between these two domains, thereby acting as a barrier. Most morphogen receptors are basolateral (facing away from the media). Hence, when cells are cultured so that at least one side rests or attaches to a surface, those cells are rendered partially or completely inaccessible to signals present in the media. Although individual free floating cells may survive briefly in suspension, they do not survive for long. Cells can be cultured for a while as aggregates in suspension but the same problems exist for aggregated cells as for cells maintained on solid surfaces-tight junctions are present on the apical sides of aggregated cells. Even when aggregated cells are disassociated, the tight junctions will quickly reassemble upon reaggregation of the cells. Aggregated cells therefore have the same barrier/receptor access problems as cells cultured on solid surfaces-morphogens in the media are not taken up, or only occasionally take up, because the tight junctions on the apical surfaces block such uptake. Under standard culture conditions using culture plates, or using flasks with cells maintained in suspension, cellular differentiation is heterogeneous because stochastic signal pathway activation occurs.

Reducing the inhibiting tight junction formation or bypassing tight junctions or as described herein, for example by ZO1 knockdown or by basolateral stimulation (e.g., by growing cells on a transwell), provides homogeneous and sustained signal pathway activation. Such reduction/removal of tight junctions is useful because signal pathway activation in the cells can specifically be controlled. The culture methods described herein therefore optimize the PGC differentiation, providing the least expensive and fastest differentiation protocol to generate PGCs.

### Basolateral BMP for Generating Primordial Germ Cells

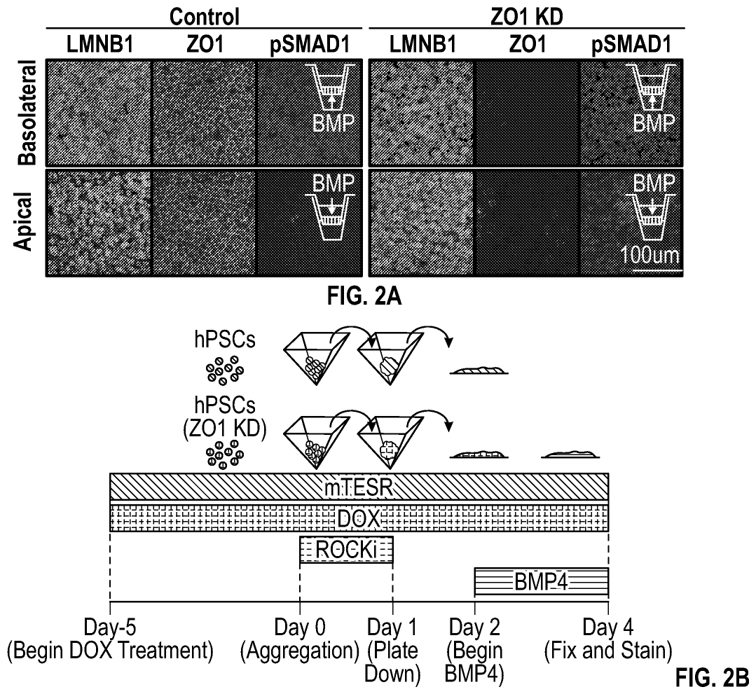

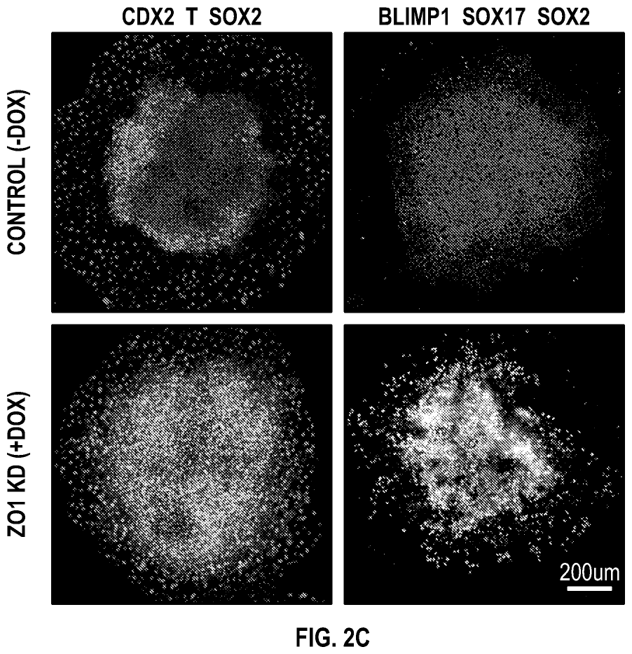

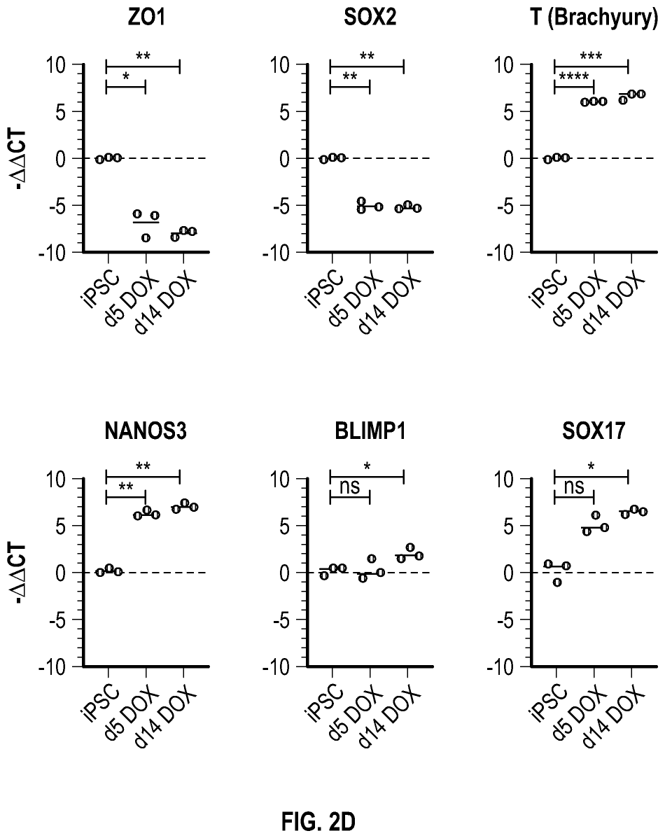

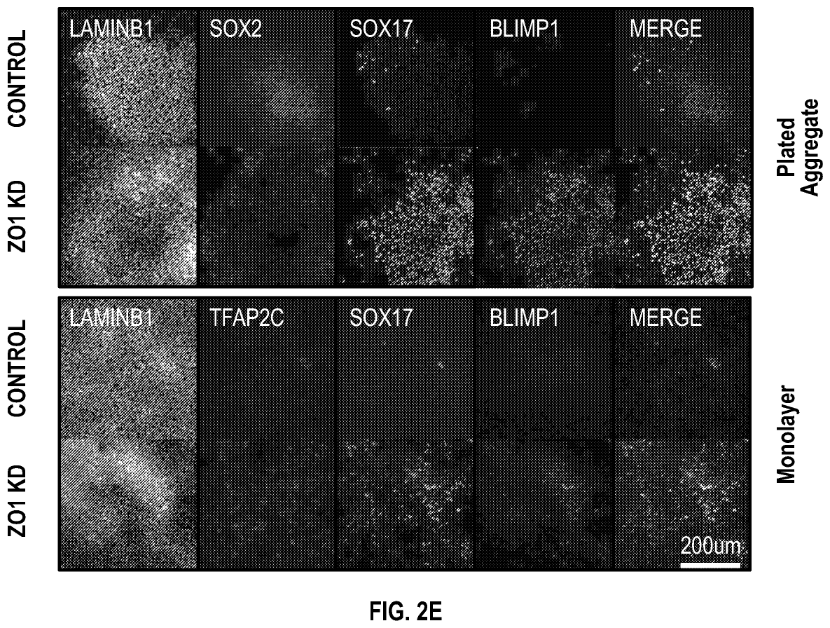

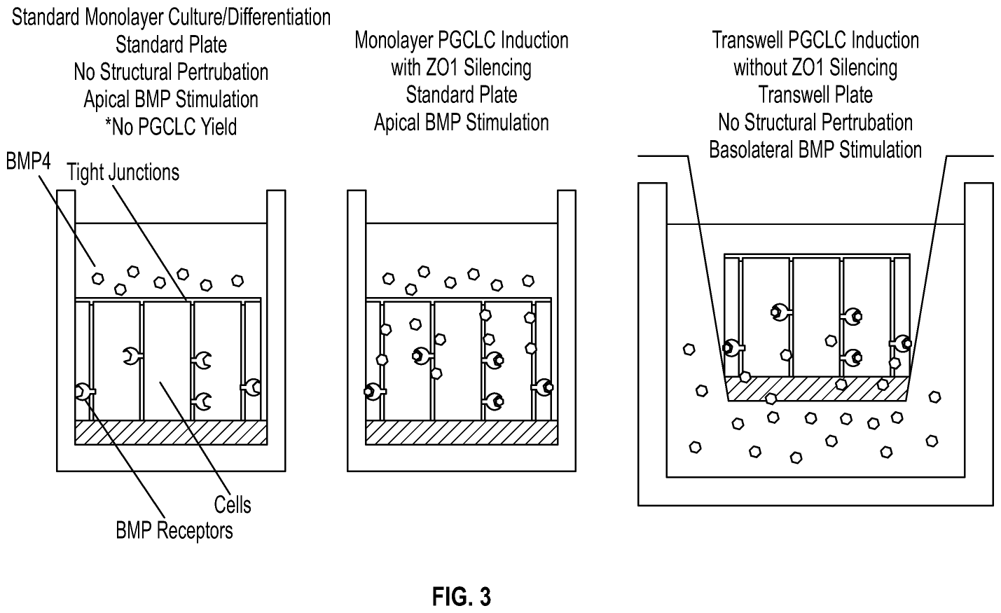

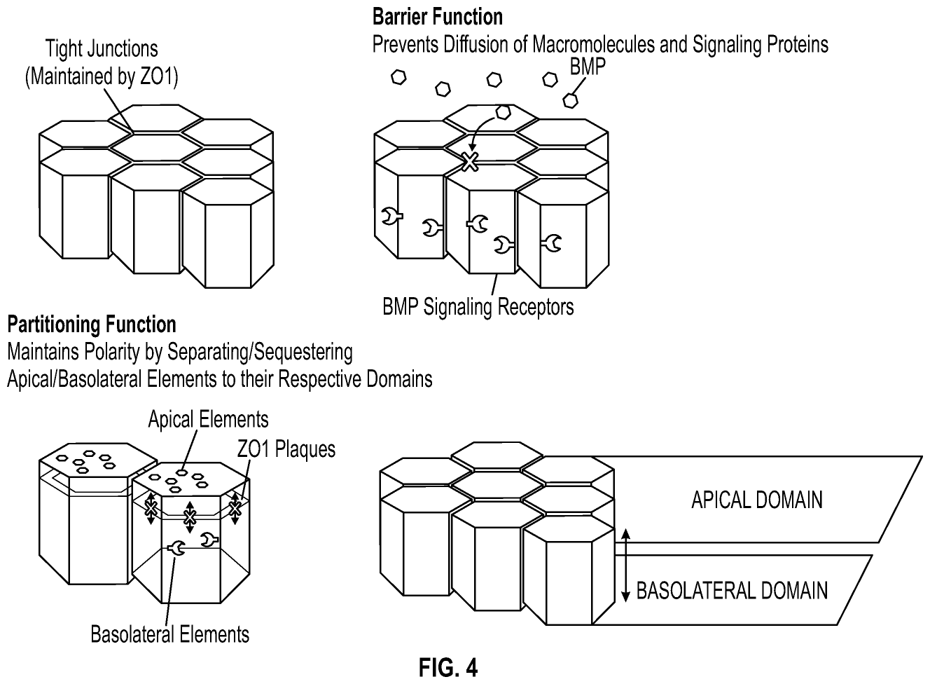

In their developmental trajectory from naïve to primed, pluripotent stem cells within the epiblast undergo epithelialization. Epithelialization is a dramatic structural change resulting in transformation of the apolar and largely disorganized mass of naïve PSCs in the inner cell mass (ICM) or early epiblast into a flat sheet-like structure (an epithelium). However, cultured cells that are in such a sheet-like structure, or in a monolayer, are less accessible to components in the culture medium (e.g., as shown in FIG. 3-4). Currently available methods typically involve contacting the apical surface of cellular monolayers. However, such methods are not effective for generating primordial germ cells, due to low activation of the canonical BMP-SMAD1 pathway (FIG. 2A).

As described herein, primordial germ cells can be generated from human induced pluripotent stem cells (e.g., hiPSCs) by incubating the PSCs in vessels that allow BMP to basolaterally contact the PSCs. A variety of pluripotent stem cells can be used, including induced pluripotent stem cells (iPSCs), embryonic stem cells, embryonic stem cells made by somatic cell nuclear transfer (ntES cells), or embryonic stem cells from unfertilized eggs (parthenogenesis embryonic stem cells, pES cells).

As used herein, the apical cell surface refers to the surface of a monolayer of cells that faces the culture medium. The apical surface does not include the cell surface that contacts the culture plate or the culture vessel or that contacts an aggregated cell mass.

As used herein, the basolateral cell surface refers to everything below the apical surface that can freely contact cell media. Hence the basolateral cell surface does not include the sides or the surfaces upon which the cells rest or that contact a solid surface or an aggregated cell mass. When cells are grown/maintained in a monolayer, the basolateral surface does not include the base of the cells that rest on a solid surface, or where the cells are laterally in contact with each other. The cell base and the cell apical surfaces are generally on opposite sides of the cells.

When generating primordial germ cells using the methods described herein, the base of the PSCs can rest upon a porous surface. The porous surface supports the cells. The porous surface can have pores of any pore size so long as the cells cannot pass through the pores. An example of a pore size range that can be used is about 0.4 μm to about 8.0 μm. Such a porous surface can be a membrane.

For example, culture medium containing BMP can be placed in a vessel or in wells of a culture plate. A membrane (e.g., transwell insert) can then be added and the PSCs can be seeded onto the membrane (e.g., of a transwell plate compartment). The cell medium below the cells (the basolateral compartment) therefore contains BMP.

In some cases the membrane can be conditioned prior to use. For example, the membrane can be incubated with extracellular matrix protein (e.g., Matrigel), and the extracellular matrix protein can be removed (e.g., by aspiration) from the membrane prior to seeding the PSCs onto the membrane.

The PSCs can be seeded at various densities. For example, the PSCs can be seeded at cell densities of about 10 cells/mm² to 10,000 cells/mm², or about 100 cells/mm² to 9,000 cells/mm², or about 200 cells/mm² to 8,000 cells/mm², or about 400 cells/mm² to 6,000 cells/mm², or about 500 cells/mm² to 5,000 cells/mm². In some cases, the PSCs can be seeded at cell densities of at least about 100 cells/mm², or at least about 300 cells/mm², or at least about 700 cells/mm².

A variety of primed pluripotent cell culture medias can be used. Examples include mTESR, MEF conditioned media, StemFit, StemPro, or E8.

The culture media used in the apical compartment need not contain BMP. However, the culture media used in the basolateral compartment does contain BMP2, BMP4, or a combination thereof. Depending on pore size of the transwell membranes used, BMP4 can sometimes diffuse to the apical compartment, however this does not affect PGCLC differentiation.

The BMP can be used in the basolateral culture media in various amounts. For example, BMP can be included in the basolateral culture media in amounts of at least 0.1 ng/ml, or at least 1 ng/ml, or at about 2 ng/ml or at least 5 ng/ml, or at least 10 ng/ml, or at least 20 ng/ml, or at least 25 ng/ml, or at least 30 ng/ml, or at least 35 ng/ml, or at least 40 ng/ml, or at least 50 ng/ml. In general, the BMP is used in the culture media in amounts less than 200 ng/ml, or less than 150 ng/ml, or less than 100 ng/ml, or less than 75 ng/ml, or less than 60 ng/ml.

The time for conversion of starting PSCs into primordial germ cells in the BMP-containing media can vary. For example, the starting cells can be incubated in vessels that provide basolateral BMP for at least about 1 day, or for at least about 2 days, or for at least about 3 days, or for at least about 4 days, or for at least about 5 days, or for at least about 6 days, or for at least about 7 days, or for at least about 8 days, or for at least about 9 days, or for at least about 10 days, or for at least about 11 days, or for at least about 12 days, or for at least about 13 days, or for at least about 14 days.

Use of BMP in contact with the basolateral sides of cells modifies epithelial structures those cells to thereby facilitate their differentiation into primordial germ cells.

Human Induced Pluripotent Stem Cells (hiPSCs)

As described herein a variety of different sources or types of pluripotent stem cells can be used to generate primordial stem cells. However, in some cases induced pluripotent stem cells (iPSCs) can be used.

Cells for that are used generating iPSCs are collected from a subject and referred to herein as “starting cells.” A selected starting population of cells may be derived from essentially any source and may be heterogeneous or homogeneous. The term “selected cell” or “selected cells” is also used to refer to starting cells. In certain embodiments, the selected starting cells to be treated as described herein are adult cells, including essentially any accessible adult cell type(s). In other embodiments, the selected starting cells treated according to the invention are adult stem cells, progenitor cells, or somatic cells. In some embodiments, the starting population of cells does not include pluripotent stem cells. In other embodiments, the starting population of cells can include pluripotent stem cells. Accordingly, a starting population of cells that is reprogrammed by the compositions and/or methods described herein, can be essentially any live cell type, particularly a somatic cell type.

The starting cells can be treated for a time and under conditions sufficient to convert the starting cells across lineage and/or differentiation boundaries to form induced pluripotent stem cells (iPSCs). Induced pluripotent stem cells are reprogrammed mature cells that have the capacity to differentiate into different mature cell type.

The starting cells can be induced to form pluripotent stem cells using either genetic or chemical induction methods. Examples of methods for generating human induced pluripotent stem cells include those described by U.S. Pat. No. 8,058,065 (Yamanaka et al.), WO/2019/165988 by Pei et al., and U.S. Patent Application No. 20190282624 by Deng et al. Induced PSC can also be generated through chemical reprogramming, via JNK pathway inhibition as illustrated by Guan et al. (Nature 605:325-331 (2022)).

The iPSCs so obtained can be incubated in any convenient primed pluripotent media. Examples of culture media that can be used include mTESR, MEF conditioned media, StemFit, StemPro, E8, and others.

A ROCK inhibitor can be used in the iPSC culture medium, especially prior to incubation with BMP. The ROCK inhibitor can be Y-27632, which is a cell-permeable, highly potent and selective inhibitor of Rho-associated, coiled-coil containing protein kinase (ROCK). Y-27632 inhibits both ROCK1 (Ki=220 nM) and ROCK2 (Ki=300 nM). A structure for Y-27632 is shown below.

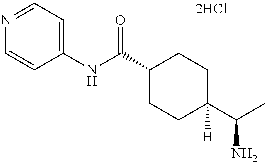 [SMILES: C[C@@H](N)[C@H]1CC[C@H](C(=O)Nc2ccncc2)CC1]

Use of Y-27632 can improve survival of stem cells when they are dissociated to single cells and after thawing the stem cells. Y-27632 can also reduce or block apoptosis of stem cells.

The ROCK inhibitor can be used in the culture media in amounts of at least 0.5 uM, or at least 1.0 uM, or at least 2.0 uM, or at least 3.0 uM, or at least 4.0 uM, or at least 5.0 uM, or at least 6.0 uM, or at least 7.0 uM, or at least 8.0 uM, or at least 9.0 uM, or at about 10 uM. In general, the ROCK inhibitor is used in the culture media in amounts less than 30 uM, or less than 25 uM, or less than 20 uM, or less than 15 uM.

The ROCK inhibitor can be used in the culture media when the hiPSCs are initially seeded into the vessel (e.g., wells) where the primordial germ cells will be generated. However, the ROCK inhibitor can be removed when the culture media is replaced with media containing BMP.

### Inhibiting Tight Junction Proteins

Epithelial structures are maintained by tight junctions, via key tight junction scaffolding proteins, such as the Zonula-occludens (ZO) family of proteins. Tight junctions form dual-purpose adhesion plaques that endow an epithelium with both barrier and partitioning functions (polarity/directionality) (see FIG. 4). Disruption of epithelial tissue structure and apical/basolateral polarity specifically, as illustrated herein, is a key method for generating primordial germ cells.

In some cases, tight junction proteins in the PSCs can be inhibited or modified (knocked down or knocked out) to facilitate generation of primordial germ cells. For example, the PSCs or incipient mesoderm-like cells (iMeLCs) can first be genetically modified or pre-treated with a tight junction inhibitor and then the cells can be cultured with BMP. As proof of principle, experiments described herein show that treatment of adherent cultures of ZO1/TIP1 knockdown cells with BMP-4 for 48 hours yielded high numbers of PGC like-cells (PGCLCs).

Examples of tight junction inhibitors that can be used include PTPN1 (Tyrosine-protein phosphatase non-receptor type 1), acetylaldehyde, genistein, protein phosphatase 2 (PP2), Clostridium perfringens enterotoxins (and their derived mutants), monoclonal antibodies against Claudin-1 (75A, OM-7D3-B3, 3A2, 6F6), monoclonal antibodies against Claudin-6 (IMAB027), Claudin-2 (1A2), monoclonal antibodies against Claudin-5 (R9, R2, 2B12), monoclonal antibodies against Occludin (1-3, 67-2), and combinations thereof.

Chelators can also be used as tight junction inhibitors, including calcium chelators. In some cases one or more of the following chelators can be used: chelator is ethylenediaminetetraacetic acid (EDTA), ethylene glycol-bis(β-aminoethyl ether)-N,N,N′,N′-tetraacetic acid (EGTA), dimercaptosuccinic acid, dimercaprol, or a combination thereof.

In some cases, tight junction proteins can be knocked down or knocked out before BMP treatment to facilitate generation of primordial germ cells. Examples of tight junction genes or tight junction proteins to be modified, inhibited, knocked down or knocked out include zonula occludens-1 (ZO1), zonula occludens-2 (ZO2), zonula occludens-3 (ZO3), OCLN, CLDN2, CLDN5, CLDN6, CLDN7. Pluripotent stem cells primarily express ZO1.

The following provides information about some tight junction genes and gene products that can be modified to reduce their expression or functioning.

### Zonula Occludens

Silencing of ZO-1 is sufficient to disrupt the epithelial structure of the pluripotent stem cells. Such epithelial structure serves two purposes: (a) to form a barrier that shields cells from the external (apical) signaling milieu and prevent paracellular diffusion of macromolecules, and (b) to sequester apical/basolateral intracellular components to their respective domains. Therefore, disruption leads to (a) increases in accessibility of the external (apical) signaling milieu to the cells/signaling receptors and (b) loss of sequestration of apical/basolateral cellular components.

Loss of ZO1 results in increased sensitivity to the morphogen BMP4, leading to more uniform and prolonged activation of the downstream signaling effector pSMAD1/5. As a result of this change in pSMAD1 signaling dynamics, treatment of adherent cultures of ZO1 knockdown (KD) cells with BMP-4 for 48 hours yields high numbers of PGC like-cells (PGCLCs), which is a name for in vitro derived PGCs that are transcriptionally similar to PGCs derived from human embryos.

ZO1 loss at the border between the epiblast and the extraembryonic ectoderm (ExE) in mice has been demonstrated to heighten activation of pSMAD1/5 in that location (Zhang et al. Nat. Commun. 2019), correlating to the location of future PGC specification (Irie et al., Reprod. Med. Biol. 2014).

The human ZO1 (TJP1) gene is located on chromosome 15 (location 15q13.1; NC_000015.10 (29699367 . . . 29969049, complement; NC_060939.1 (27490136 . . . 27760675, complement). An amino acid sequence for a human zonula occludens-1 (ZO1) polypeptide is available in the NCBI and UNIPROT databases (NCBI accession no. Q07157.3; UNIPROT accession no. Q07157) and shown below as SEQ ID NO:1.

| | |
|---|---|
| 1 | MSARAAAAKS TAMEETAIWE QHTVTLHRAP GFGFGIAISG |
| 41 | GRDNPHFQSG ETSIVISDVL KGGPAEGQLQ ENDRVAMVNG |
| 81 | VSMDNVEHAF AVQQLRKSGK NAKITIRRKK KVQIPVSRPD |
| 121 | PEPVSDNEED SYDEEIHDPR SGRSGVVNRR SEKIWPRDRS |
| 161 | ASRERSLSPR SDRRSVASSQ PAKPTKVTLV KSRKNEEYGL |
| 201 | RLASHIFVKE ISQDSLAARD GNIQEGDVVL KINGTVTENM |
| 241 | SLTDAKTLIE RSKGKLKMVV QRDERATLLN VPDLSDSIHS |
| 281 | ANASERDDIS EIQSLASDHS GRSHDRPPRR SRSRSPDQRS |
| 321 | EPSDHSRHSP QQPSNGSLRS RDEERISKPG AVSTPVKHAD |
| 361 | DHTPKTVEEV TVERNEKQTP SLPEPKPVYA QVGQPDVDLP |
| 401 | VSPSDGVLPN STHEDGILRP SMKLVKFRKG DSVGLRLAGG |
| 441 | NDVGIFVAGV LEDSPAAKEG LEEGDQILRV NNVDFTNIIR |
| 481 | EEAVLFLLDL PKGEEVTILA QKKKDVYRRI VESDVGDSFY |
| 521 | IRTHEEYEKE SPYGLSFNKG EVFRVVDTLY NGKLGSWLAI |
| 561 | RIGKNHKEVE RGIIPNKNRA EQLASVQYTL PKTAGGDRAD |
| 601 | FWRFRGLRSS KRNLRKSRED LSAQPVQTKF PAYERVVLRE |
| 641 | AGFLRPVTIF GPIADVAREK LAREEPDIYQ IAKSEPRDAG |
| 681 | TDQRSSGIIR LHTIKQIIDQ DKHALLDVTP NAVDRLNYAQ |
| 721 | WYPIVVFLNP DSKQGVKTMR MRLCPESRKS ARKLYERSHK |
| 761 | LRKNNHHLFT TTINLNSMND GWYGALKEAI QQQQNQLVWV |
| 801 | SEGKADGATS DDLDLHDDRL SYLSAPGSEY SMYSTDSRHT |
| 841 | SDYEDTDTEG GAYTDQELDE TLNDEVGTPP ESAITRSSEP |
| 881 | VREDSSGMHH ENQTYPPYSP QAQPQPIHRI DSPGFKPASQ |
| 921 | QKAEASSPVP YLSPETNPAS STSAVNHNVN LTNVRLEEPT |
| 961 | PAPSTSYSPQ ADSLRTPSTE AAHIMLRDQE PSLSSHVDPT |
| 1001 | KVYRKDPYPE EMMRQNHVLK QPAVSHPGHR PDKEPNLTYE |
| 1041 | PQLPYVEKQA SRDLEQPTYR YESSSYTDQF SRNYEHRLRY |
| 1081 | EDRVPMYEEQ WSYYDDKQPY PSRPPFDNQH SQDLDSRQHP |
| 1121 | EESSERGYFP RFEEPAPLSY DSRPRYEQAP RASALRHEEQ |
| 1161 | PAPGYDTHGR LRPEAQPHPS AGPKPAESKQ YFEQYSRSYE |
| 1201 | QVPPQGFTSR AGHFEPLHGA AAVPPLIPSS QHKPEALPSN |
| 1241 | TKPLPPPPTQ TEEEEDPAMK PQSVLTRVKM FENKRSASLE |
| 1281 | TKKDVNDTGS FKPPEVASKP SGAPIIGPKP TSQNQFSEHD |
| 1321 | KTLYRIPEPQ KPQLKPPEDI VRSNHYDPEE DEEYYRKQLS |
| 1361 | YFDRRSFENK PPAHIAASHL SEPAKPAHSQ NQSNFSSYSS |
| 1401 | KGKPPEADGV DRSFGEKRYE PIQATPPPPP LPSQYAQPSQ |
| 1441 | PVTSASLHIH SKGAHGEGNS VSLDFQNSLV SKPDPPPSQN |
| 1448 | KPATFRPPNR EDTAQAAFYP QKSFPDKAPV NGTEQTQKTV |
| 1521 | TPAYNRFTPK PYTSSARPFE RKFESPKFNH NLLPSETAHK |
| 1561 | PDLSSKTPTS PKTLVKSHSL AQPPEFDSGV ETFSIHAEKP |
| 1601 | KYQINNISTV PKAIPVSPSA VEEDEDEDGH TVVATARGIF |
| 1641 | NSNGGVLSSI ETGVSIIIPQ GAIPEGVEQE IYFKVCRDNS |
| 1681 | ILPPLDKEKG ETLLSPLVMC GPHGLKFLKP VELRLPHCDP |
| 1721 | KTWQNKCLPG DPNYLVGANC VSVLIDHF |

The TJP1 gene encodes the ZO1 polypeptide with SEQ ID NO: 1. The TIPI gene is on chromosome 15 (location 15q13.1; NC_000015.10 (29699367 . . . 29969049, complement). A nucleotide sequence that encodes the ZO1 polypeptide with SEQ ID NO: 1 is available as European Nucleotide Archive accession no. L14837, provided below as SEQ ID NO:2.

| | |
|---|---|
| 1 | TCCGGGTATG GATGTCAATC TTTTGTCTAC AATGTGAATA |
| 41 | CATTTATCCT TCGGGGACCA TCAAGACTTT CAGGAAAGGC |
| 81 | CCCGCCTGTC TCTGCGCGGC CACTTTGCTG GGACAAAGGT |
| 121 | CAACTGAAGA AGTGGGCAGG CCCGAGGCAG GAGAGATGCT |
| 161 | GAGGAGTCCA TGTGCAGGGG AGGGAAAGGG AGAGGCAGTC |
| 201 | AGGGAGAGGA GGAGGAGGTA CCGCCAGAAG GGGATCCTCC |
| 241 | CGCTCCGAAA ACCAGACACC GGGTCTTGCC CTGTGGTCCA |
| 281 | GGCAGGAGTG CAGTGGTGCA ACCTCAGCTC ACTGCAGCCT |
| 321 | TGACCTCCCC GGGCTCAAGC GATCCTCCGG CCACAGCACT |
| 361 | TGGCTGTTCA GCGGCTGGAG GAGCAGGGCC CCAGGTCCTC |
| 401 | CCCACCCTCA CCTGCTGCTC CCAGGTCGTG GCCGTCTTGC |
| 441 | TCTTCCAGGT CCTTCTCTAG GGATGCAATA TTCACATTGC |
| 481 | TAAGATGCAG GTCTAACGCA GAACCTGTCA ACAGAGCCCC |
| 521 | CCATCATCCA CAGCCCACCC AGCGCTGCAG AGCTCAGGAA |
| 561 | GCCTAGCTGA GGAGGACGAC CGTCCCACCT GGGCTTAGAG |
| 601 | TGAGACCAAG GGCAGAAGGC GTGGGAGTTG CTGGGGCAGC |
| 641 | CAGGGAAGGA CACCCCCAGC CCGTCCTCGC AGCCCCCCAC |
| 681 | AGGCAGTGGG AGGCTTGGCT GTTCCTCCGG CAAAACGGGC |
| 721 | ATGCTCAGTG GGCCGGGCCG GCAGGTTTGC GTGGCCGCTG |
| 761 | AGTTGCCGGC GCCGGCTGAG CCAGCGGACG CCGCGTTCCT |
| 801 | TGGCGGCCGC CGGTTCCCGG GAAGTTACGT GGCGAAGCCG |
| 841 | GCTTCCGAGG AGACGCCGGG AGGCCACGGG TGCTGCTGAC |
| 881 | GGGCGGGCGA CCGGGCGAGG CCGACGTGGC CGGGCTGCGA |
| 921 | AAGCTGCGGG AGGCCGAGTG GGTGACCGCG CTCGGAGGGA |
| 961 | GGTGCCGGTC GGGCGCGCCC CGTGGAGAAG ACCCGGGGGG |
| 1001 | GGCGGGCGCT TCCCGGACTT TTGTCCGAGT TGAATTCCCT |
| 1041 | CCCCCTGGGC CGGGCCCTTC CGTCCGCCCC CGCCCGTGCC |
| 1081 | CCGCTCGCTC TCGGGAGATG TTTATTTGGG CTGTGGCGTG |
| 1121 | AGGAGCGGGG GGGCCAGCGC CGCGGAGTTT CGGGTCCGAG |
| 1161 | GAGCCTCGCG CGGCGCTGGA GAGAGACAAG ATGTCCGCCA |
| 1201 | GAGCTGCGGC CGCCAAGAGC ACAGCAATGG AGGAAACAGC |
| 1241 | TATATGGGAA CAACATACAG TGACGCTTCA CAGGGCTCCT |
| 1281 | GGATTTGGAT TTGGAATTGC AATATCTGGT GGACGAGATA |
| 1321 | ATCCTCATTT TCAGAGTGGG GAAACGTCAA TAGTGATTTC |
| 1361 | AGATGTGCTG AAAGGAGGAC CAGCTGAAGG ACAGCTACAG |
| 1401 | GAAAATGACC GAGTTGCAAT GGTTAACGGA GTTTCAATGG |
| 1441 | ATAATGTTGA ACATGCTTTT GCTGTTCAGC AACTAAGGAA |
| 1481 | AAGTGGGAAA AATGCAAAAA TTACAATTAG AAGGAAGAAG |
| 1521 | AAAGTTCAAA TACCAGTAAG TCGTCCTGAT CCTGAACCAG |
| 1561 | TATCTGATAA TGAAGAAGAT AGTTATGATG AGGAAATACA |
| 1601 | TGATCCAAGA AGTGGCCGGA GTGGTGTGGT TAACAGAAGG |
| 1641 | AGTGAGAAGA TTTGGCCGAG GGATAGAAGT GCAAGTAGAG |
| 1681 | AGAGGAGCTT GTCCCCGCGG TCAGACAGGC GGTCAGTGGC |
| 1721 | TTCCAGCCAG CCTGCTAAAC CTACTAAAGT CACACTGGTG |
| 1761 | AAATCCCGGA AAAATGAAGA ATATGGTCTT CGATTGGCAA |
| 1801 | GCCATATATT TGTTAAGGAA ATTTCACAAG ATAGTTTGGC |
| 1841 | AGCAAGAGAT GGCAATATTC AAGAAGGTGA TGTTGTATTG |
| 1881 | AAGATAAATG GTACTGTGAC AGAAAATATG TCATTGACAG |
| 1921 | ATGCAAAGAC ATTGATAGAA AGGTCTAAAG GCAAATTAAA |
| 1961 | AATGGTAGTT CAAAGAGATG AACGGGCTAC GCTATTGAAT |
| 2001 | GTCCCTGATC TTTCTGACAG CATCCACTCT GCTAATGCCT |
| 2041 | CTGAGAGAGA CGACATTTCA GAAATTCAGT CACTGGCATC |
| 2081 | AGATCATTCT GGTCGATCAC ACGATAGGCC TCCCCGCCGC |
| 2121 | AGCCGGTCAC GATCTCCTGA CCAGCGGTCA GAGCCTTCTG |
| 2161 | ATCATTCCAG GCACTCGCCG CAGCAGCCAA GCAATGGCAG |
| 2201 | TCTCCGGAGT AGAGATGAAG AGAGAATTTC TAAACCTGGG |
| 2241 | GCTGTCTCAA CTCCTGTAAA GCATGCTGAT GATCACACAC |
| 2281 | CTAAAACAGT GGAAGAAGTT ACAGTTGAAA GAAATGAGAA |
| 2321 | ACAAACACCT TCTCTTCCAG AACCAAAGCC TGTGTATGCC |
| 2361 | CAAGTTGGCA ACCAGATGTG GATTTACCTG TCAGTCCATC |
| 2401 | TGATGGTGTC CTACCTAATT CAACTCATGA AGATGGGATT |
| 2441 | TCTTCGGCCC AGCATGAAAT TGGTAAAATT CAGAAAAGGA |
| 2481 | GATAGTGTGG GTTTGCGGCT GGCTGGTGGA AATGATGTTG |
| 2521 | GAATATTTGT AGCTGGCGTT CTAGAAGATA GCCCTGCAGC |
| 2561 | CAAGGAAGGC TTAGAGGAAG GTGATCAAAT TCTCAGGGTA |
| 2601 | AACAACGTAG ATTTTACAAA TATCATAAGA GAAGAAGCCG |
| 2641 | TCCTTTTCCT GCTTGACCTC CCTAAAGGAG AAGAAGTGAC |
| 2681 | CATATTGGCT CAGAAGAAGA AGGATGTTTA TCGTCGCATT |
| 2721 | GTAGAATCAG ATGTAGGAGA TTCTTTCTAT ATTAGAACCC |
| 2761 | ATTTTGAATA TGAAAAGGAA TCTCCCTATG GACTTAGTTT |
| 2801 | TAACAAAGGA GAGGTGTTCC GTGCTGTGGA TACCTTGTAC |
| 2841 | AATGGAAAAC TGGGCTCTTG GCTTGCTATT CGAATTGGTA |
| 2881 | AAAATCATAA GGAGGTAGAA CGAGGCATCA TCCCTAATAA |
| 2921 | GAACAGAGCT GAGCAGCTAG CCAGTGTACA GTATACACTT |
| 2961 | CCAAAAACAG CAGGCGGAGA CCGTGCTGAC TTCTGGAGAT |
| 3001 | TCAGAGGTCT TCGCAGCTCC AAGAGAAATC TTCGAAAAAG |
| 3041 | CAGAGAGGAT TTGTCCGCTC AGCCTGTTCA AACAAAGTTT |
| 3081 | CCAGCTTATG AAAGAGTGGT TCTTCGAGAA GCTGGATTTC |
| 3121 | TGAGGCCTGT AACCATTTTT GGACCAATAG CTGATGTTGC |
| 3161 | CAGAGAAAAG CTGGCAAGAG AAGAACCAGA TATTTATCAA |
| 3201 | ATTGCAAAGA GTGAACCACG AGACGCTGGA ACTGACCAAC |
| 3241 | GTAGCTCTGG CTATATTCGC CTGCATACAA TAAAGCAAAT |
| 3281 | CATAGATCAA GACAAACATG CTTTATTAGA TGTAACACCA |
| 3321 | AATGCAGTTG ATCGTCTTAA CTATGCCCAG TGGTATCCAA |
| 3361 | TTGTTGTATT TCTTAACCCT GATTCTAAGC AAGGAGTAAA |
| 3401 | AACAATGAGA ATGAGGTTAT GTCCAGAATC TCGGAAAAGT |
| 3441 | GCCAGGAAGT TATACGAGCG ATCTCATAAA CTTGCTAAAA |
| 3481 | ATAATCACCA TCTTTTTACA ACTACAATTA ACTTAAATTC |
| 3521 | AATGAATGAT GGTTGGTATG GTGCGCTGAA AGAAGCAGTT |
| 3561 | CAACAACAGC AAAACCAGCT GGTATGGGTT TCCGAGGGAA |
| 3601 | AGGCGGATGG TGCTACAAGT GATGACCTTG ATTTGCATGA |
| 3641 | TGATCGTCTG TCCTACCTGT CAGCTCCAGG TAGTGAATAC |
| 3681 | TCAATGTATA GCACGGACAG TAGACACACT TCTGACTATG |
| 3721 | AAGACACAGA CACAGAAGGC GGGGCCTACA CTGATCAAGA |
| 3761 | ACTAGATGAA ACTCTTAATG ATGAGGTTGG GACTCCACCG |
| 3801 | GAGTCTGCCA TTACACGGTC CTCTGAGCCT GTAAGAGAGG |
| 3841 | ACTCCTCTGG AATGCATCAT GAAAACCAAA CATATCCTCC |
| 3881 | TTACTCACCA CAAGCGCAGC CACAACCAAT TCATAGAATA |
| 3921 | GACTCCCCTG GATTTAAGCC AGCCTCTCAA CAGAAAGCAG |
| 3961 | AAGCTTCATC TCCAGTCCCT TACCTTTCGC CTGAAACAAA |
| 4001 | CCCAGCATCA TCAACCTCTG CTGTTAATCA TAATGTAAAT |
| 4041 | TTAACTAATG TCAGACTGGA GGAGCCCACC CCAGCTCCTT |
| 4081 | CCACCTCTTA CTCACCACAA GCTGATTCTT TAAGAACACC |
| 4121 | AAGTACTGAG GCAGCTCACA TAATGCTAAG AGATCAAGAA |
| 4161 | CCATCATTGT CGTCGCATGT AGATCCAACA AAGGTGTATA |
| 4201 | GAAAGGATCC ATATCCCGAG GAAATGATGA GGCAGAACCA |
| 4241 | TGTTTTGAAA CAGCCAGCCG TTAGTCACCC AGGGCACAGG |
| 4281 | CCAGACAAAG AGCCTAATCT GACCTATGAA CCCCAACTCC |
| 4321 | CATACGTAGA GAAACAAGCC AGCAGAGACC TCGAGCAGCC |
| 4361 | CACATACAGA TACGAGTCCT CAAGCTATAC GGACCAGTTT |
| 4401 | TCTCGAAACT ATGAACATCG TCTGCGATAC GAAGATCGCG |
| 4441 | TCCCCATGTA TGAAGAACAG TGGTCATATT ATGATGACAA |
| 4481 | ACAGCCCTAC CCATCTCGGC CACCTTTTGA TAATCAGCAC |
| 4521 | TCTCAAGACC TTGACTCCAG ACAGCATCCC GAAGAGTCCT |
| 4561 | CAGAACGAGG GTACTTTCCA CGTTTTGAAG AGCCAGCCCC |
| 4601 | TCTGTCTTAC GACAGCAGAC CACGTTACGA ACAGGCACCT |
| 4641 | AGAGCATCCG CCCTGCGGCA CGAAGAGCAG CCAGCTCCTG |
| 4681 | GGTATGACAC ACATGGTAGA CTCAGACCGG AAGCCCAGCC |
| 4721 | CCACCCTTCA GCAGGGCCCA AGCCTGCAGA GTCCAAGCAG |
| 4761 | TATTTTGAGC AATATTCACG CAGTTACGAG CAAGTACCAC |
| 4801 | CCCAAGGATT TACCTCTAGA GCAGGTCATT TTGAGCCTCT |
| 4841 | CCATGGTGCT GCAGCTGTCC CTCCGCTGAT ACCTTCATCT |
| 4881 | CAGCATAAGC CAGAAGCTCT GCCTTCAAAC ACCAAACCAC |
| 4921 | TGCCTCCACC CCCAACTCAA ACCGAAGAAG AGGAAGATCC |
| 4961 | AGCAATGAAG CCACAGTCTG TACTCACCAG AGTTAAGATG |
| 5001 | TTTGAAAACA AAAGATCTGC ATCCTTAGAG ACCAAGAAGG |
| 5041 | ATGTAAATGA CACTGGCAGT TTTAAGCCTC CAGAAGTAGC |
| 5081 | ATCTAAACCT TCAGGTGCTC CCATCATTGG TCCCAAACCC |
| 5121 | ACTTCTCAGA ATCAATTCAG TGAACATGAC AAAACTCTGT |
| 5161 | ACAGGATCCC AGAACCTCAA AAACCTCAAC TGAAGCCACC |
| 5201 | TGAAGATATT GTTCGGTCCA ATCATTATGA CCCTGAAGAA |
| 5241 | GATGAAGAAT ATTATCGAAA ACAGCTGTCA TACTTTGACC |
| 5281 | GAAGAAGTTT TGAGAATAAG CCTCCTGCAC ACATTGCTGC |
| 5321 | CAGCCATCTC TCCGAGCCTG CAAAGCCAGC TCATTCTCAG |
| 5361 | AATCAATCAA ATTTTTCTAG TTATTCTTCA AAGGGAAAGC |
| 5401 | CTCCTGAAGC TGATGGTGTG GATAGATCAT TTGGCGAGAA |
| 5441 | ACGCTATGAA CCCATCCAGG CCACTCCCCC TCCTCCTCCA |
| 5481 | TTGCCCTCGC AGTATGCCCA GCCATCTCAG CCTGTCACCA |
| 5521 | GCGCGTCTCT CCACATACAT TCTAAGGGAG CACATGGTGA |
| 5561 | AGGTAATTCA GTGTCATTGG ATTTTCAGAA TTCCTTAGTG |
| 5601 | TCCAAACCAG ACCCACCTCC ATCTCAGAAT AAGCCAGCAA |
| 5641 | CTTTCAGACC ACCAAACCGA GAAGATACTG CTCAGGCAGC |
| 5681 | TTTCTATCCC CAGAAAAGTT TTCCAGATAA AGCCCCAGTT |
| 5721 | AATGGAACTG AACAGACTCA GAAAACAGTC ACTCCAGCAT |
| 5761 | ACAATCGATT CACACCAAAA CCATATACAA GTTCTGCCCG |
| 5801 | ACCATTTGAA CGCAAGTTTG AAAGTCCTAA ATTCAATCAC |
| 5841 | AATCTTCTGC CAAGTGAAAC TGCACATAAA CCTGACTTGT |
| 5881 | CTTCAAAAAC TCCCACTTCT CCAAAAACTC TTGTGAAATC |
| 5921 | GCACAGTTTG GCACAGCCTC CTGAGTTTGA CAGTGGAGTT |
| 5961 | GAAACTTTCT CTATCCATGC AGAGAAGCCT AAATATCAAA |
| 6001 | TAAATAATAT CAGCACAGTG CCTAAAGCTA TTCCTGTGAG |
| 6041 | TCCTTCAGCT GTGGAAGAGG ATGAAGATGA AGATGGTCAT |
| 6081 | ACTGTGGTGG CCACAGCCCG AGGCATATTT AACAGCAATG |
| 6121 | GGGGCGTGCT GAGTTCCATA GAAACTGGTG TTAGTATAAT |
| 6161 | TATCCCTCAA GGAGCCATTC CCGAAGGAGT TGAGCAGGAA |
| 6201 | ATCTATTTCA AGGTCTGCCG GGACAACAGC ATCCTTCCAC |
| 6241 | CTTTAGATAA AGAGAAAGGT GAAACACTGC TGAGTCCTTT |
| 6281 | GGTGATGTGT GGTCCCCATG GCCTCAAGTT CCTGAAGCCT |
| 6321 | GTGGAGCTGC GCTTACCACA CTGTGATCCT AAAACCTGGC |
| 6361 | AAAACAAGTG TCTTCCCGGA GATCCAAATT ATCTCGTTGG |
| 6401 | AGCAAACTGT GTTTCTGTCC TTATTGACCA CTTTTAACTC |
| 6441 | TTGAAATATA GGAACTTAAA TAATGTGAAA CTGGATTAAA |
| 6481 | CTTAATCTAA ATGGAACCAC TCTATCAAGT ATTATACCTT |
| 6521 | TTTTAGAGTT GATACTACAG TTTGTTAGTA TGAGGCATTT |
| 6561 | GTTTGAACTG ATAAAGATGA GTGAGCATGC CCCTGAACCA |
| 6601 | TGGTCGGAAA ACATGCTACA CACTGCATGT TTGTGATTGA |
| 6641 | CGGGACTGTT GGTATTGGCT AGAGGTTCAA AGATATTTTG |
| 6681 | CTTTGTGATT TTTGTAATTT TTTTATCGTC ACTGCTTAAC |
| 6721 | TTCACATATT GATTTCCGTT AAAATACCAG CCAGTAAATG |
| 6761 | GGGGTGCATT TGAGGTCTGT TCTTTCCAAA GTACACTGTT |
| 6801 | TCAAACTTTA CTATGGCCCT GGCCTAGCAT ACGTACACAT |
| 6841 | TTTATTTTAT TATGCATGAA GTAATATGCA CACATTTTTT |
| 6881 | AAATGCACCT GGAATATATA ACCAGTGTTG TGGATTTAAC |
| 6921 | AGAAATGTAC AGCAAGGAGA TTTACAACTG GGGGAGGGTG |
| 6961 | AAGTGAAGAC AATGACTTAC TGTACATGAA AACACATTTT |
| 7001 | TCTTAGGGAA GGATACAAAA GCATGTGAGA CTGGTTCCAT |
| 7041 | GGCCTCTTCA GATCTCTAAC TTCACCATAT TACCACAGAC |
| 7081 | ATACTAACCA GCAGAAATGC CTTACCCTCA TGTTCTTAAT |
| 7121 | TCTTAGCTCA TTCTCCTTGT GTTACTAAGT TTTTATGGCT |
| 7161 | TTTGTGCATT ATCTAGATAC TGTATCATGA CAAAGACTGA |
| 7201 | GTACGTTGTG CATTTGGTGG TTTCAGAAAT GTGTTATCAC |
| 7241 | CCAGAAGAAA ATAGTGGTGT GATTTGGGGA TATTTTTTTC |
| 7281 | TTTTCTTTTC TTTTCTTTTT TTTTTTTTTT TGACAAGGGG |
| 7321 | CAGTGGTGGT TTTCTGTTCT TTCTGGCTAT GCATTTGAAA |
| 7361 | ATTTTGATGT TTTAAGGATG CTTGTACATA ATGCGTGCAT |
| 7401 | ACCACTTTTG TTCTTGGTTT GTAAATTAAC TTTTATAAAC |
| 7441 | TTTACCTTTT TTATACATAA ACAAGACCAC GTTTCTAAAG |
| 7481 | GCTACCTTTG TATTCTCTCC TGTACCTCTT GAGCCTTGAA |
| 7521 | CTTTGACCTC TGCAGCAATA AAGCAGCGTT TCTATGACAC |
| 7561 | ATGCAAGGTC ATTTTTTTTA AGAAAAAGGA TGCACAGAGT |
| 7601 | TGTTACATTT TTAAGTGCTG CATTTAAAAG ATACAGTTAC |
| 7641 | TCAGAATTCT CTAGTTTGAT TAAATTCTTG CAAAGTATCC |
| 7681 | CTACTGTAAT TTGTGATACA ATGCTGTGCC CTAAAGTGTA |
| 7721 | TTTTTTTACT AATAGACAAT TTATTATGAC ACATCAGCAC |
| 7761 | GATTTCTGTT TAAATAATAC ACCACTACAT TCTGTTAATC |
| 7800 | ATTAGGTGTG ACTGAATTTC TTTTGCCGTT ATTAAAAATC |
| 7841 | TCAAATTTCT AAATCTCCAA AATAAAACTT TTTAAAATAA |
| 7881 | AAAAAAAT |

An amino acid sequence for a human zonula occludens-2 (Z (2) polypeptide is available in the NCBI and UNIPROT databases (NCBI accession no. Q9UDY2.2; UNIPROT accession no. Q9UDY2) and shown below as SEQ ID NO:3.

| | |
|---|---|
| 1 | MPVRGDRGFP PRRELSGWLR APGMEELIWE QYTVTLQKDS |
| 41 | KRGFGIAVSG GRDNPHFENG ETSIVISDVL PGGPADGLLQ |
| 81 | ENDRVVMVNG TPMEDVLHSF AVQQLRKSGK VAAIVVKRPR |
| 121 | KVQVAALQAS PPLDQDDRAF EVMDEFDGRS FRSGYSERSR |
| 161 | LNSHGGRSRS WEDSPERGRP HERARSRERD LSRDRSRGRS |
| 201 | LERGLDQDHA RTRDRSRGRS LERGLDHDFG PSRDRDRDRS |
| 241 | RGRSIDQDYE RAYHRAYDPD YERAYSPEYR RGARHDARSR |
| 281 | GPRSRSREHP HSRSPSPEPR GRPGPIGVLL MKSRANEEYG |
| 321 | LRLGSQIFVK EMTRTGLATK DGNLHEGDII LKINGTVTEN |
| 361 | MSLTDARKLI EKSRGKLQLV VLRDSQQTLI NIPSLNDSDS |
| 401 | EIEDISEIES NRSFSPEERR HQYSDYDYHS SSEKLKERPS |
| 441 | SREDTPSRLS RMGATPTPEK STGDIAGTVV PETNKEPRYQ |
| 481 | EDPPAPQPKA APRTFLRPSP EDEAIYGPNT KMVRFKKGDS |
| 521 | VGLRLAGGND VGIFVAGIQE GTSAEQEGLQ EGDQILKVNT |
| 561 | QDFRGLVRED AVLYLLEIPK GEMVTILAQS RADVYRDILA |
| 601 | CGRGDSFFIR SHFECEKETP QSLAFTRGEV FRVVDTLYDG |
| 641 | KLGNWLAVRI GNELEKGLIP NKSRAEQMAS VQNAQRDNAG |
| 681 | DRADFWRMRG QRSGVKKNLR KSREDLTAVV SVSTKFPAYE |
| 721 | RVLLREAGFK RPVVLEGPIA DIAMEKLANE LPDWFQTAKT |
| 761 | EPKDAGSEKS TGVVRLNTVR QIIEQDKHAL LDVTPKAVDL |
| 801 | LNYTQWFPIV IFFNPDSRQG VKTMRQRLNP TSNKSSRKLF |
| 841 | DQANKLKKTC AHLFTATINL NSANDSWFGS LKDTIQHQQG |
| 881 | EAVWVSEGKM EGMDDDPEDR MSYLTAMGAD YLSCDSRLIS |
| 921 | DFEDTDGEGG AYTDNELDEP AEEPLVSSIT RSSEPVQHEE |
| 961 | SIRKPSPEPR AQMRRAASSD QLRDNSPPPA FKPEPPKAKT |
| 1001 | QNKEESYDFS KSYEYKSNPS AVAGNETPGA STKGYPPPVA |
| 1041 | AKPTFGRSIL KPSTPIPPQE GEEVGESSEE QDNAPKSVLG |
| 1081 | KVKIFEKMDH KARLQRMQEL QEAQNARIEI AQKHPDIYAV |
| 1121 | PIKTHKPDPG TPQHTSSRPP EPQKAPSRPY QDTRGSYGSD |
| 1161 | AEEEEYRQQL SEHSKRGYYG QSARYRDTEL |

The TJP2 gene encodes the ZO2 polypeptide with SEQ ID NO:3. The TJP2 gene is on chromosome 9 (location NC_000009.12 (69121006 . . . 69255208)). A nucleotide sequence that encodes the ZO2 polypeptide with SEQ ID NO:3 is available as European Nucleotide Archive accession no. L27476, provided below as SEQ ID NO: 4.

| | |
|---|---|
| 1 | TGCCCAGGAG GAGTAGGAGC AGGAGCAGAA GCAGAAGCGG |
| 41 | GGTCCGGAGC TGCGCGCCTA CGCGGGACCT GTGTCCGAAA |
| 81 | TGCCGGTGCG AGGAGACCGC GGGTTTCCAC CCCGGCGGGA |
| 121 | GCTGTCAGGT TGGCTCCGCG CCCCAGGCAT GGAAGAGCTG |
| 161 | ATATGGGAAC AGTACACTGT GACCCTACAA AAGGATTCCA |
| 201 | AAAGAGGATT TGGAATTGCA GTGTCCGGAG GCAGAGACAA |
| 241 | CCCCCACTTT GAAAATGGAG AAACGTCAAT TGTCATTTCT |
| 281 | GATGTGCTCC CGGGTGGGCC TGCTGATGGG CTGCTCCAAG |
| 321 | AAAATGACAG AGTGGTCATG GTCAATGGCA CCCCCATGGA |
| 361 | GGATGTGCTT CATTCGTTTG CAGTTCAGCA GCTCAGAAAA |
| 401 | AGTGGGAAGG TCGCTGCTAT TGTGGTCAAG AGGCCCCGGA |
| 441 | AGGTCCAGGT GGCCGCACTT CAGGCCAGCC CTCCCCTGGA |
| 481 | TCAGGATGAC CGGGCTTTTG AGGTGATGGA CGAGTTTGAT |
| 521 | GGCAGAAGTT TCCGGAGTGG CTACAGCGAG AGGAGCCGGC |
| 561 | TGAACAGCCA TGGGGGGCGC AGCCGCAGCT GGGAGGACAG |
| 601 | CCCGGAAAGG GGGCGTCCCC ATGAGCGGGC CCGGAGCCGG |
| 641 | GAGCGGGACC TCAGCCGGGA CCGGAGCCGT GGCCGGAGCC |
| 681 | TGGAGCGGGG CCTGGACCAA GACCATGCGC GCACCCGAGA |
| 721 | CCGCAGCCGT GGCCGGAGCC TGGAGCGGGG CCTGGACCAC |
| 761 | GACTTTGGGC CATCCCGGGA CCGGGACCGT GACCGCAGCC |
| 801 | GCGGCCGGAG CATTGACCAG GACTACGAGC GAGCCTATCA |
| 841 | CCGGGCCTAC GACCCAGACT ACGAGCGGGC CTACAGCCCG |
| 881 | GAGTACAGGC GCGGGGCCCG CCACGATGCC CGCTCTCGGG |
| 921 | GACCCCGAAG CCGCAGCCGC GAGCACCCGC ACTCACGGAG |
| 961 | CCCCAGCCCC GAGCCTAGGG GGCGGCCGGG GCCCATCGGG |
| 1001 | GTCCTCCTGA TGAAAAGCAG AGCGAACGAA GAGTATGGTC |
| 1041 | TCCGGCTTGG GAGTCAGATC TTCGTAAAGG AAATGACCCG |
| 1081 | AACGGGTCTG GCAACTAAAG ATGGCAACCT TCACGAAGGA |
| 1121 | GACATAATTC TCAAGATCAA TGGGACTGTA ACTGAGAACA |
| 1161 | TGTCTTTAAC GGATGCTCGA AAATTGATAG AAAAGTCAAG |
| 1201 | AGGAAAACTA CAGCTAGTGG TGTTGAGAGA CAGCCAGCAG |
| 1241 | ACCCTCATCA ACATCCCGTC ATTAAATGAC AGTGACTCAG |
| 1281 | AAATAGAAGA TATTTCAGAA ATAGAGTCAA CCCGATCATT |
| 1321 | TTCTCCAGAG GAGAGACGTC ATCAGTATTC TGATTATGAT |
| 1361 | TATCATTCCT CAAGTGAGAA GCTGAAGGAA AGGCCAAGTT |
| 1401 | CCAGAGAGGA CACGCCGAGC AGATTGTCCA GGATGGGTGC |
| 1441 | GACACCCACT CCCTTTAAGT CCACAGGGGA TATTGCAGGC |
| 1481 | ACAGTTGTCC CAGAGACCAA CAAGGAACCC AGATACCAAG |
| 1521 | AGGAACCCCC AGCTCCTCAA CCAAAAGCAG CCCCGAGAAC |
| 1561 | TTTTCTTCGT CCTAGTCCTG AAGATGAAGC AATATATGGC |
| 1601 | CCTAATACCA AAATGGTAAG GTTCAAGAAG GGAGACAGCG |
| 1641 | TGGGCCTCCG GTTGGCTGGT GGCAATGATG TCGGGATATT |
| 1681 | TGTTGCTGGC ATTCAAGAAG GGACCTCGGC GGAGCAGGAG |
| 1721 | GGCCTTCAAG AAGGAGACCA GATTCTGAAG GTGAACACAC |
| 1761 | AGGATTTCAG AGGATTAGTG CGGGAGGATG CCGTTCTCTA |
| 1801 | CCTGTTAGAA ATCCCTAAAG GTGAAATGGT GACCATTTTA |
| 1841 | GCTCAGAGCC GAGCCGATGT GTATAGAGAC ATCCTGGCTT |
| 1881 | GTGGCAGAGG GGATTCGTTT TTTATAAGAA GCCACTTTGA |
| 1921 | ATGTGAGAAG GAAACTCCAC AGAGCCTGGC CTTCACCAGA |
| 1961 | GGGGAGGTCT TCCGAGTGGT AGACACACTG TATGACGGCA |
| 2001 | AGCTGGGCAA CTGGCTGGCT GTGAGGATTG GGAACGAGTT |
| 2041 | GGAGAAAGGC TTAATCCCCA ACAAGAGCAG AGCTGAACAA |
| 2081 | ATGGCCAGTG TTCAAAATGC CCAGAGAGAC AACGCTGGGG |
| 2121 | ACCGGGCAGA TTTCTGGAGA ATGCGTGGCC AGAGGTCTGG |
| 2161 | GGTGAAGAAG AACCTGAGGA AAAGTCGGGA AGACCTCACA |
| 2201 | GCTGTTGTGT CTGTCAGCAC CAAGTTCCCA GCTTATGAGA |
| 2241 | GGGTTTTGCT GCGAGAAGCT GGTTTCAAGA GACCTGTGGT |
| 2281 | CTTATTCGGC CCCATAGCTG ATATAGCAAT GGAAAAATTG |
| 2321 | GCTAATGAGT TACCTGACTG GTTTCAAACT GCTAAAACGG |
| 2361 | AACCAAAAGA TGCAGGATCT GAGAAATCCA CTGGAGTGGT |
| 2401 | CCGGTTAAAT ACCGTGAGGC AAGTTATTGA ACAGGATAAG |
| 2441 | CATGCACTAC TGGATGTGAC TCCGAAAGCT GTGGACCTGT |
| 2481 | TGAATTACAC CCAGTGGTTC TCAATTGTGA TTTCTTTCAC |
| 2521 | GCCAGACTCC AGACAAGGTG TCAACACCAT GAGACAAAGG |
| 2561 | TTAGACCCAA CGTCCAACAA TAGTTCTCGA AAGTTATTTG |
| 2601 | ATCACGCCAA CAAGCTTAAA AAAACGTGTG CACACCTTTT |
| 2641 | TACAGCTACA ATCAACCTAA ATTCAGCCAA TGATAGCTGG |
| 2681 | TTTGGCAGCT TAAAGGACAC TATTCAGCAT CAGCAAGGAG |
| 2721 | AAGCGGTTTG GGTCTCTGAA GGAAAGATGG AAGGGATGGA |
| 2761 | TGATGACCCC GAAGACCGCA TGTCCTACTT AACTGCCATG |
| 2801 | GGCGCAGACT ATCTGAGTTG CGACAGCCGC CTCATCAGTG |
| 2841 | ACTTTGAAGA CACGGACGGT GAAGGAGGCG CCTACACTGA |
| 2881 | CAATGAGCTG GATGAGCCAG CCGAGGAGCC GCTGGTGTCG |
| 2921 | TCCATCACCC GCTCCTCGGA GCCGGTGCAG CACGAGGAGA |
| 2961 | GCATAAGGAA ACCCAGCCCA GAGCCACGAG CTCAGATGAG |
| 3001 | GAGGGCTGCT AGCAGCGATC AACTTAGGGA CAATAGCCCG |
| 3041 | CCCCCAGCAT TCAAGCCAGA GCCGTCCAAG GCCAAAACCC |
| 3081 | AGAACAAAGA AGAATCCTAT GACTTCTCCA AATCCTATGA |
| 3121 | ATATAAGTCA AACCCCTCTG CCGTTGCTGG TAATGAAACT |
| 3161 | CCTGGGGCAT CTACCAAAGG TTATCCTCCT CCTGTTGCAG |
| 3201 | CAAAACCTAC CTTTGGGGGG TCTATACTGA AGCCCTCCAC |
| 3241 | TCCCATCCCT CCTCAAGAGG GTGAGGAGGT GGGAGAGAGC |
| 3281 | AGTGAGGAGC AAGATAATGC TCCCAAATCA GTCCTGGGCA |
| 3321 | AAGTCAAAAT ATTTGGAGAA GATGGATCAC AAGGGCCAGG |
| 3361 | GTTACAAGAG AATGCAGGAG CTCCAGGAAG CACAGAATGC |
| 3401 | AAGGATCGAA ATTGCCCAGA AGCATCCTGA TATCTATGCA |
| 3441 | GTTCCAATCA AAACGCACAA GCCAGACCCT GGCACGCCCC |
| 3481 | AGCACACGAG TTCCAGACCC CCTGAGCCAC AGAAAGCTCC |
| 3521 | TTCCAGACCT TATCAGGATA CCAGAGGAAG TTATGGCAGT |
| 3561 | GATGCCGAGG AGGAGGAGTA CCGCCAGCAG CTGTCAGAAC |
| 3601 | ACTCCAAGCG CGGTTACTAT GGCCAGTCTG CCCGATACCG |
| 3641 | GGACACAGAA TTATAGATGT CTGAGCACGG ACTCTCCCAG |
| 3681 | GCCTGCCTGC ATGGCATCAG ACTAGCCACT CCTGCCAGGC |
| 3721 | CGCCGGGATG GTTCTTCTCC AGTTAGAATG CACCATGGAG |
| 3761 | ACGTGGTGGG ACTCCAGCTC GTGTGTCCTC ATGGAGAACC |
| 3801 | CAGGGGACAG CTGGTGCAAA TTCAGAACTG AGGGCTCTGT |
| 3841 | TTGTGGGACT GGGTTAGAGG AGTCTGTGGC TTTTTGTTCA |
| 3881 | GAATTAAGCA GAACACTGCA GTCAGATCCT GTTACTTGCT |
| 3921 | TCAGTGGACC GAAATCTGTA TTCTGTTTGC GTACTTGTAA |
| 3961 | TATGTATATT AAGAAGCAAT AACTATTTTT CCTCATTAAT |
| 4001 | AGCTGCCTTC AAGGACTGTT TCAGTGTGAG TCAGAATGTG |
| 4041 | AAAAAGGAAT AAAAAATACT GTTGGGCTCA AACTAAATTC |
| 4081 | AAAGAAGTAC TTTATTGCAA CTCTTTTAAG TGCCTTGGAT |
| 4121 | GAGAAGTGTC TTAAATTTTC TTCCTTTGAA GCTTTAGGCA |
| 4161 | GAGCCATAAT GGACTAAAAC ATTTTGACTA AGTTTTTATA |
| 4201 | CCAGCTTAAT AGCTGTAGTT TTCCCTGCAC TGTGTCATCT |
| 4241 | TTTCAAGGCA TTTGTCTTTG TAATATTTTC CATAAATTTG |
| 4281 | GACTGTCTAT ATCATAACTA TACTTGATAG TTTGGCTATA |
| 4321 | AGTGCTCAAT AGCTTGAAGC CCAAGAAGTT GGTATCGAAA |
| 4361 | TTTGTTGTTT GTTTAAACCC AAGTGCTGCA CAAAAGCAGA |
| 4401 | TACTTGAGGA AAACACTATT TCCAAAAGCA CATGTATTGA |
| 4441 | CAACAGTTTT ATAATTTAAT AAAAAGGAAT ACATTGCAAT |
| 4481 | CCGT |

An amino acid sequence for a human zonula occludens-3 (ZO3) polypeptide is available in the NCBI and UNIPROT databases (NCBI accession no. EAW69293.1; UNIPROT accession no. 095049) and shown below as SEQ ID NO:5.

| | |
|---|---|
| 1 | MEELTIWEQH TATLSKDPRR GFGIAISGGR DRPGGSMVVS |
| 41 | DVVPGGPAEG RLQTGDHIVM VNGVSMENAT SAFAIQILKT |
| 81 | CTKMANITVK RPRRIHLPAT KASPSSPGRQ DSDEDDGPQR |
| 121 | VEEVDQGRGY DGDSSSGSGR SWDERSRRPR PGRRGRAGSH |
| 161 | GRRSPGGGSE ANGLALVSGF KRLPRQDVQM KPVKSVLVKR |
| 201 | RDSEEFGVKL GSQIFIKHIT DSGLAARHRG LQEGDLILQI |
| 241 | NGVSSQNLSL NDTRRLIEKS EGKLSLLVLR DRGQFLVNIP |
| 281 | PAVSDSDSSP LEEGVTMADE MSSPPADISD LASELSQAPP |
| 321 | SHIPPPPRHA QRSPEASQTD SPVESPRLRR ESSVDSRTIS |
| 361 | EPDEQRSELP RESSYDIYRV PSSQSMEDRG YSPDTRVVRF |
| 401 | LKGKSIGLRL AGGNDVGIFV SGVQAGSPAD GQGIQEGDQI |
| 441 | LQVNDVPFQN LTREEAVQFL LGLPPGEEME LVTQRKQDIF |
| 481 | WKMVQSRVGD SFYIRTHFEL EPSPPSGLGF TRGDVFHVLD |
| 521 | TLHPGPGQSH ARGGHWLAVR MGRDLREQER GIIPNQSRAE |
| 561 | QLASLEAAQR AVGVGPGSSA GSNARAEFWR LRGLRRGAKK |
| 601 | TTQRSREDLS ALTRQGRYPP YERVVLREAS FKRPVVILGP |
| 641 | VADIAMQKLT AEMPDQFEIA ETVSRTDSPS KIIKLDTVRV |
| 681 | IAEKDKHALL DVTPSAIERL NYVQYYPIVV FFIPESRPAL |
| 721 | KALRQWLAPA SRRSTRRLYA QAQKLRKHSS HLFTATIPLN |
| 761 | GTSDTWYQEL KAIIREQQTR PIWTAEDQLD GSLEDNLDLP |
| 801 | HHGLADSSAD LSCDSRVNSD YETDGEGGAY TDGEGYTDGE |
| 841 | GGPYTDVDDE PPAPALARSS EPVQADESQS PRDRGRISAH |
| 881 | QGAQVDSRHP QGQWRQDSMR TYEREALKKK FMRVHDAESS |
| 921 | DEDGYDWGPA TDL |

The TJP3 gene encodes the ZO3 polypeptide with SEQ ID NO:5. The TJP3 gene is on chromosome 19 (location NC_000019.10 (3708384 . . . 3750813)). A nucleotide sequence that encodes the ZO3 polypeptide with SEQ ID NO:5 is available as European Nucleotide Archive accession no. AK091118, provided below as SEQ ID NO: 6.

| | |
|---|---|
| 1 | AGTTCCACTG GCAGGCGACC TGCCTCCCTG TTGCCACCAC |
| 41 | AAGAGAGGAA AAGTTGGTCA AACAGGTGGG GAGGCCAGAG |
| 61 | CTACAAGCCT CGGGTTCCCT CCCCACCACC CGTGCCAGGC |
| 121 | AGGCACCCGG GCCCTGGCAC CTGCTGCCTG CCCAGAGGCC |
| 161 | ACCCAGCCTC CTAGACAGGT GGCTGACATG GAGGAGCTGA |
| 201 | CCATCTGGGA ACAGCACACG GCCACACTGT CCAAGGACCC |
| 241 | CCGCCGGGGC TTTGGCATTG CGATCTCTGG AGGCCGAGAC |
| 281 | CGGCCCGGTG GATCCATGGT TGTATCTGAC GTGGTACCTG |
| 321 | GAGGGCCGGC GGAGGGCAGG CTACAGACAG GCGACCACAT |
| 361 | TGTCATGGTG AACGGGGTTT CCATGGAGAA TGCCACCTCC |
| 401 | GCGTTTGCCA TTCAGATACT CAAGACCTGC ACCAAGATGG |
| 441 | CCAACATCAC AGTGAAACGT CCCCGGAGGA TCCTCCTGCC |
| 481 | CGCCACCAAA GCCAGCCCCT CCAGCCCAGG GCGCCAGGAC |
| 521 | TCGGATGAAG ACGATGGGCC CCAGCGGGTG GAGGAGGTGG |
| 561 | ACCAGGGCCG GGGCTATGAC GGCGACTCAT CCAGTGGCTC |
| 601 | CGGCCGCTCC TGGGACGAGC GCTCCCGCCG GCCGAGGCCT |
| 641 | GGTCGCCGGG GCCGGGCCGG CAGCCATGGG CGTAGGAGCC |
| 681 | CAGGTGGTGG CTCTGAGGCC AACGGGCTGG CCCTGGTGTC |
| 721 | CGGCTTTAAG CGGCTGCCAC GGCAGGACGT GCAGATGAAG |
| 761 | CCTGTGAAGT CAGTGCTGGT GAAGAGGAGA GACAGCGAAG |
| 801 | AGTTTGGCGT CAAGCTGGGC AGTCAGATCT TCATCAAGCA |
| 841 | CATTACAGAT TCGGGCCTGG CTGCCCGGCA CCGTGGGCTG |
| 881 | CAGGAAGGAG ATCTCATTCT ACAGATCAAC GGGGTGTCTA |
| 921 | GCCAGAACCT GTCACTGAAC GACACCCGGC GACTGATTGA |
| 961 | GAAGTCAGAA GGGAAGCTAA GCCTGCTGGT GCTGAGAGAT |
| 1001 | CGTGGGCAGT TCCTGGTGAA CATTCCGCCT GCTGTCAGTG |
| 1041 | ACAGCGACAG CTCGCCATTG GAGGACATCT CGGACCTCGC |
| 1081 | CTCGGAGCTA TCGCAGGCAC CACCATCCCA CATCCCACCA |
| 1121 | CCACCCCGGC ATGCTCAGCG GAGCCCCGAG GCCAGCCAGA |
| 1161 | CCGACTCTCC CGTGGAGAGT CCCCGGCTTC GGCGGGAAAG |
| 1201 | TTCAGTAGAT TCCAGAACCA TCTCGGAACC AGATGAGCAA |
| 1241 | CGGTCAGAGT TGCCCAGGGA AAGCAGCTAT GACATCTACA |
| 1281 | GAGTGCCCAG CAGTCAGAGC ATGGAGGATC GTGGGTACAG |
| 1321 | CCCCGACACG CGTGTGGTCC GCTTCCTCAA GGGCAAGAGC |
| 1361 | ATCGGGCTGC GGCTGGCAGG GGGCAATGAC GTGGGCATCT |
| 1401 | TCGTGTCCGG GGTGCAGGCG GGCAGCCCGG CCGACGGGCA |
| 1441 | GGGCATCCAG GAGGGAGATC AGATTCTGCA GGTGAATGAC |
| 1481 | GTGCCATTCC AGAACCTGAC ACGGGAGGAG GCAGTGCAGT |
| 1521 | TCCTGCTGGG GCTGCCACCA GGCGAGGAGA TGGAGCTGGT |
| 1561 | GACGCAGAGG AAGCAGGACA TTTTCTGGAA AATGGTGCAG |
| 1601 | TCCCGCGTGG GTGACTCCTT CTACATCCGC ACTCACTTTG |
| 1641 | AGCTGGAGCC CAGTCCACCG TCTGGCCTGG GCTTCACCCG |
| 1681 | TGGCGACGTC TTCCACGTGC TGGACACGCT GCACCCCGGC |
| 1721 | CCCGGGCAGA GCCACGCACG AGGAGGCCAC TGGCTGGCGG |
| 1761 | TGCGCATGGG TCGTGACCTG CGGGAGCAAG AGCGGGGCAT |
| 1801 | CATTCCCAAC CAGAGCAGGG CGGAGCAGCT GGCCAGCCTG |
| 1841 | GAAGCTGCCC AGAGGGCCGT GGGAGTCGGG CCCGGCTCCT |
| 1881 | CCGCGGGCTC CAATGCTCGG GCCGAGTTCT GGCGGCTGCG |
| 1921 | GGGTCTTCGT CGAGGAGCCA AGAAGACCAC TCAGCGGAGC |
| 1961 | CGTGAGGACC TCTCAGCTCT GACCCGACAG GGCCGCTACC |
| 2001 | CGCCCTACGA ACGAGTGGTG TTGCGAGAAG CCAGTTTCAA |
| 2041 | GCGCCCGGTA GTGATCCTGG GACCCGTGGC CGACATTGCT |
| 2081 | ATGCAGAAGT TGACTGCTGA GATGCCTGAC CAGTTTGAAA |
| 2121 | TCGCAGAGAC TGTGTCCAGG ACCGACAGCC CCTCCAAGAT |
| 2161 | CATCAAACTA GACACCGTGC GGGTGATTGC AGAAAAAGAC |
| 2201 | AAGCATGCGC TCCTGGATGT GACCCCCTCC GCCATCGAGC |
| 2241 | GCCTCAACTA TGTGCAGTAC TACCCCATTG TGGTCTTCTT |
| 2281 | CATCCCCGAG AGCCGGCCGG CCCTCAAGGC ACTGCGCCAG |
| 2321 | TGGCTGGCGC CTGCCTCCCG CCGCAGCACC CGTCGCCTCT |
| 2361 | ACGCACAAGC CCAGAAGCTG CGAAAACACA GCAGCCACCT |
| 2401 | CTTCACAGCC ACCATCCCTC TGAATGGCAC GAGTGACACC |
| 2441 | TGGTACCAGG AGCTCAAGGC CATCATTCGA GAGCAGCAGA |
| 2481 | CGCGGCCCAT CTGGACGGCG GAAGATCAGC TGGATGGCTC |
| 2521 | CTTGGAGGAC AACCTAGACC TCCCTCACCA CGGCCTGGCC |
| 2561 | GACAGCTCCG CTGACCTCAG CTGCGACAGC CACGTTAACA |
| 2601 | GCGACTACGA GACGGACGGC GAGGGCGGCG CGTACACGGA |
| 2641 | TGGCGAGGGC TACACAGACG GCGAGGGGGG GCCCTACACG |
| 2681 | GATGTGGATG ATGAGCCCCC GGCTCCAGCC CTGGCCCGGT |
| 2721 | CCTCGGAGCC CGTGCAGGCA GATGAGTCCC AGAGCCCGAG |
| 2761 | GGATCGTGGG AGAATCTCGG CTCATCAGGG GGCCCAGGTG |
| 2801 | GACAGCCGCC ACCCCCAGGG ACAGTGGCGA CAGGACAGCA |
| 2841 | TGCGAACCTA TGAACGGGAA GCCCTGAAGA AAAAGTTTAC |
| 2881 | GCGAGTCCGT GATGCGGAGT CCTCCGATGA AGACGGCTAT |
| 2921 | GACTGGGGTC CGGCCACTGA CCTGTGACCT CTCGCAGGCT |
| 2961 | GCCAGCTGGT CCGTCCTCCT TCTCCCTCCC TGGGGCTGGG |
| 3001 | ACTCAGTTTC CCATACAGAA CCCACAACCT TACCTCCCTC |
| 3041 | CGCCTGGTCT TTAATAAACA GAGTATTTTC ACAGC |

### Occludin (OCLN)

An amino acid sequence for a human OCLN polypeptide is available in the NCBI and UNIPROT databases (NCBI accession no. AAH29886; see also UNIPROT accession no. Q16625) and shown below as SEQ ID NO:7.

| | |
|---|---|
| 1 | MSSRPLESPP PYRPDEFKPN HYAPSNDIYG GEMHVRPMLS |
| 41 | QPAYSFYPED EILHFYKWTS PPGVIRILSM LIIVMCIAIF |
| 81 | ACVASTLAWD RGYGTSLLGG SVGYPYGGSG FGSYGSGYGY |
| 121 | GYGYGYGYGG YTDPRAAKGF MLAMAAFCFI AALVIFVTSV |
| 161 | IRSEMSRTRR YYLSVIIVSA ILGIMVFIAT IVYIMGVNPT |
| 201 | AQSSGSLYGS QIYALCNQFY TPAATGLYVD QYSYHYCVVD |
| 241 | PQEAIAIVLG FMIIVAFALI IFFAVKTRRK MDRYDKSNIL |
| 281 | WDKEHIYDEQ PPNVEEWVKN VSAGTQDVPS PPSDYVERVD |
| 321 | SPMAYSSNGK VNDKRFYPES SYKSTPVPEV VQELPLTSPV |
| 361 | DDFRQPRYSS GGNFETPSKR APAKGRAGRS KRTEQDHYET |
| 401 | DYTTGGESCD ELEEDWIREY PPITSDQQRQ LYKRNFDTGL |
| 441 | QEYKSLQSEL DEINKELSRL DKELDDYREE SEEYMAAADE |
| 481 | YNRLKQVKGS ADYKSKKNHC KQLKSKLSHI KKMVGDYDRQ |
| 521 | KT |

The OCLN gene encodes the OCLN polypeptide with SEQ ID NO:7. The OCLN gene is on chromosome 5 (location NC_000005.10 (69492547 . . . 69558104)). A nucleotide sequence that encodes the OCLN polypeptide with SEQ ID NO:7 is available as NCBI accession no. NG_028291.1. A cDNA sequence encoding the polypeptide having UNIPROT accession no. Q16625 is available as European Nucleotide Archive accession no. U49184, provided below as SEQ ID NO:8.

| | |
|---|---|
| 1 | CTCCCGCGTC CACCTCTCCC TCCCTGCTTC CTCTGGCGGA |
| 41 | GGCGGCAGGA ACCGAGAGCC AGGTCCAGAG CGCCGAGGAG |
| 81 | CCGGTCTAGG ACGCAGCAGA TTGGTTTATC TTGGAAGCTA |
| 121 | AAGGGCATTG CTCATCCTGA AGATCAGCTG ACCATTGACA |
| 161 | ATCAGCCATG TCATCCAGGC CTCTTGAAAG TCCACCTCCT |
| 201 | TACAGGCCTG ATGAATTCAA ACCGAATCAT TATGCACCAA |
| 241 | GCAATGACAT ATATGGTGGA GAGATGCATG TTCGACCAAT |
| 281 | GCTCTCTCAG CCAGCCTACT CTTTTTACCC AGAAGATGAA |
| 321 | ATTCTTCACT TCTACAAATG GACCTCTCCT CCAGGAGTGA |
| 361 | TTCGGATCCT GTCTATGCTC ATTATTGTGA TGTGCATTGC |
| 401 | CATCTTTGCC TGTGTGGCCT CCACGCTTGC CTGGGACAGA |
| 441 | GGCTATGGAA CTTCCCTTTT AGGAGGTAGT GTAGGCTACC |
| 481 | CTTATGGAGG AAGTGGCTTT GGTAGCTACG GAAGTGGCTA |
| 521 | TGGCTATGGC TATGGTTATG GCTATGGCTA CGGAGGCTAT |
| 561 | ACAGACCCAA GAGCAGCAAA GGGCTTCATG TTGGCCATGG |
| 601 | CTGCCTTTTG TTTCATTGCC GCGTTGGTGA TCTTTGTTAC |
| 641 | CAGTGTTATA AGATCTGAAA TGTCCAGAAC AAGAAGATAC |
| 681 | TACTTAAGTG TGATAATAGT GAGTGCTATC CTGGGCATCA |
| 721 | TGGTGTTTAT TGCCACAATT GTCTATATAA TGGGAGTGAA |
| 761 | CCCAACTGCT CAGTCTTCTG GATCTCTATA TGGTTCACAA |
| 801 | ATATATGCCC TCTGCAACCA ATTTTATACA CCTGCAGCTA |
| 841 | CTGGACTCTA CGTGGATCAG TATTTGTATC ACTACTGTGT |
| 881 | TGTGGATCCC CAGGAGGCCA TTGCCATTGT ACTGGGGTTC |
| 921 | ATGATTATTG TGGCTTTTGC TTTAATAATT TTCTTTGCTG |
| 961 | TGAAAACTCG AAGAAAGATG GACAGGTATG ACAAGTCCAA |
| 1001 | TATTTTGTGG GACAAGGAAC ACATTTATGA TGAGCAGCCC |
| 1041 | CCCAATGTCG AGGAGTGGGT TAAAAATGTG TCTGCAGGCA |
| 1081 | CACAGGACGT GCCTTCACCC CCATCTGACT ATGTGGAAAG |
| 1121 | AGTTGACAGT CCCATGGCAT ACTCTTCCAA TGGCAAAGTG |
| 1161 | AATGACAAGC GGTTTTATCC AGAGTCTTCC TATAAATCCA |
| 1201 | CGCCGGTTCC TGAAGTGGTT CAGGAGCTTC CATTAACTTC |
| 1241 | GCCTGTGGAT GACTTCAGGC AGCCTCGTTA CAGCAGCGGT |
| 1281 | GGTAACTTTG AGACACCTTC AAAAAGAGCA CCTGCAAAGG |
| 1321 | GAAGAGCAGG AAGGTCAAAG AGAACAGAGC AAGATCACTA |
| 1361 | TGAGACAGAC TACACAACTG GCGGCGAGTC CTGTGATGAG |
| 1401 | CTGGAGGAGG ACTGGATCAG GGAATATCCA CCTATCACTT |
| 1441 | CAGATCAACA AAGACAACTG TACAAGAGGA ATTTTGACAC |
| 1481 | TGGCCTACAG GAATACAAGA GCTTACAATC AGAACTTGAT |
| 1521 | GAGATCAATA AAGAACTCTC CCGTTTGGAT AAAGAATTGG |
| 1561 | ATGACTATAG AGAAGAAAGT GAAGAGTACA TGGCTGCTGC |
| 1601 | TGATGAATAC AATAGACTGA AGCAAGTGAA GGGATCTGCA |
| 1641 | GATTACAAAA GTAAGAAGAA TCATTGCAAG CAGTTAAAGA |
| 1681 | GCAAATTGTC ACACATCAAG AAGATGGTTG GAGACTATGA |
| 1721 | TAGACAGAAA ACATAGAAGG CTGATGCCAA GTTGTTTGAG |
| 1761 | AAATTAAGTA TCTGACATCT CTGCAATCTT CTCAGAAGGC |
| 1801 | AAATGACTTT GGACCATAAC CCCGGAAGCC AAACCTCTGT |
| 1841 | GAGCATCACA AAGTTTTGGT TGCTTTAACA TCATCAGTAT |
| 1881 | TGAAGCATTT TATAAATCGC TTTTGATAAT CAACTGGGCT |
| 1921 | GAACACTCCA ATTAAGGATT TTATGCTTTA AACATTGGTT |
| 1961 | CTTGTATTAA GAATGAAATA CTGTTTGAGG TTTTTAAGCC |
| 2001 | TTAAAGGAAG GTTCTGGTGT GAACTAAACT TTCACACCCC |
| 2041 | AGACGATGTC TTCATACCTA CATGTATTTG TTTGCATAGG |
| 2081 | TGATCTCATT TAATCCTCTC AACCACCTTT CAGATAACTG |
| 2121 | TTATTTATAA TCACTTTTTT CCACATAAGG AAACTGGGTT |
| 2161 | CCTGCAATGA AGTCTCTGAA GTGAAACTGC TTGTTTCCTA |
| 2201 | GCACACACTT TTGGTTAAGT CTGTTTTATG ACTTCATTAA |
| 2241 | TAATAAATTC CCTGGCCTTT CATATTTTAG CTACTATATA |
| 2281 | TGTGATGATC TACCAGCCTC CCTATTTTTT TTCTGTTATA |
| 2321 | TAAATGGTTA AAAGAGGTTT TTCTTAAATA ATAAAGATCA |
| 2361 | TGTAAAAGTA AAAAAAAAA |

### Claudins

An amino acid sequence for a human claudin-2 (CLDN2) polypeptide is available in the NCBI and UNIPROT databases (NCBI accession no, NP_065117; see also UNIPROT accession no. P57739) and shown below as SEQ ID NO:9.

| | |
|---|---|
| 1 | MASLGLQLVG YILGLIGLLG TLVAMLLPSW KTSSYVGASI |
| 41 | VTAVGFSKGL WMECATHSTG ITQCDIYSTL LGLPADIQAA |
| 81 | QAMMVTSSAI SSLACIISVV GMRCTVFCQE SRAKDRVAVA |
| 121 | GGVFFILGGL LGFIPVAWNL HGILRDFYSP LVPDSMKFEI |
| 161 | GEALYLGIIS SLFSLIAGII LCFSCSSQRN RSNYYDAYQA |
| 201 | QPLATRSSPR PGQPPKVKSE FNSYSLTGYV |

The CLDN2 gene encodes the CLDN2 polypeptide with SEQ ID NO:9. The CLDN2 gene is on the X chromosome (location NC_000023.11 (106900164 . . . 106930861). A nucleotide sequence that encodes the CLDN2 polypeptide with SEQ ID NO:9 is available as NCBI accession no. NG_016445.1. A cDNA sequence encoding the polypeptide having NCBI accession no. NM_020384.4 is shown below as SEQ ID NO: 10,

| | |
|---|---|
| 1 | GCAGATGGAT TTTGCAAAGC TGTGGTTAAC GATTAGAAAT |
| 41 | CCTTTATCAC CTCAGCCCGT GGCCCCTTGT ACTTCGCTCC |
| 81 | CCTCCCTCAG GATCCCTTTC TCCCTCTCCA GGGGCATCTC |
| 121 | CCCCTCCAAG GCTCTGCAAA GAACTGCCCT GTCTTCTAGA |
| 161 | TGCCTTCTTG AGGCTGCTTG TGGCCACCCA CAGACACTTG |
| 201 | TAAGGAGGAG AGAAGTCAGC CTGGCAGAGA GACTCTGAAA |
| 241 | TGAGGGATTA GAGGTGTTCA AGGAGCAAGA GCTTCAGCCT |
| 281 | GAAGACAAGG GAGCAGTCCC TGAAGACGCT TCTACTGAGA |
| 321 | GGTCTGCCAT GGCCTCTCTT GGCCTCCAAC TTGTGGGCTA |
| 361 | CATCCTAGGC CTTCTGGGGC TTTTGGGCAC ACTGGTTGCC |
| 401 | ATGCTGCTCC CCAGCTGGAA AACAAGTTCT TATGTCGGTG |
| 441 | CCAGCATTGT GACAGCAGTT GGCTTCTCCA AGGGCCTCTG |
| 481 | GATGGAATGT GCCACACACA GCACAGGCAT CACCCAGTGT |
| 521 | GACATCTATA GCACCCTTCT GGGCCTGCCC GCTGACATCC |
| 561 | AGGCTGCCCA GGCCATGATG GTGACATCCA GTGCAATCTC |
| 601 | CTCCCTGGCC TGCATTATCT CTGTGGTGGG CATGAGATGC |
| 641 | ACAGTCTTCT GCCAGGAATC CCGAGCCAAA GACAGAGTGG |
| 681 | CGGTAGCAGG TGGAGTCTTT TTCATCCTTG GAGGCCTCCT |
| 721 | GGGATTCATT CCTGTTGCCT GGAATCTTCA TGGGATCCTA |
| 761 | CGGGACTTCT ACTCACCACT GGTGCCTGAC AGCATGAAAT |
| 801 | TTGAGATTGG AGAGGCTCTT TACTTGGGCA TTATTTCTTC |
| 841 | CCTGTTCTCC CTGATAGCTG GAATCATCCT CTGCTTTTCC |
| 881 | TGCTCATCCC AGAGAAATCG CTCCAACTAC TACGATGCCT |
| 921 | ACCAAGCCCA ACCTCTTGCC ACAAGGAGCT CTCCAAGGCC |
| 961 | TGGTCAACCT CCCAAAGTCA AGAGTGAGTT CAATTCCTAC |
| 1001 | AGCCTGACAG GGTATGTGTG AAGAACCAGG GGCCAGAGCT |
| 1041 | GGGGGGTGGC TGGGTCTGTG AAAAACAGTG GACAGCACCC |
| 1081 | CGAGGGCCAC AGGTGAGGGA CACTACCACT GGATCGTGTC |
| 1121 | AGAAGGTGCT GCTGAGGATA GACTGACTTT GGCCATTGGA |
| 1161 | TTGAGCAAAG GCAGAAATGG GGGCTAGTGT AACAGCATGC |
| 1201 | AGGTTGAATT GCCAAGGATG CTCGCCATGC CAGCCTTTCT |
| 1241 | GTTTTCCTCA CCTTGCTGCT CCCCTGCCCT AAGTCCCCAA |
| 1281 | CCCTCAACTT GAAACCCCAT TCCCTTAAGC CAGGACTCAG |
| 1321 | AGGATCCCTT TGCCCTCTGG TTTACCTGGG ACTCCATCCC |
| 1361 | CAAACCCACT AATCACATCC CACTGACTGA CCCTCTGTGA |
| 1401 | TCAAAGACCC TCTCTCTGGC TGAGGTTGGC TCTTAGCTCA |
| 1441 | TTGCTGGGGA TGGGAAGGAG AAGCAGTGGC TTTTGTGGGC |
| 1481 | ATTGCTCTAA CCTACTTCTC AAGCTTCCCT CCAAAGAAAC |
| 1521 | TGATTGGCCC TGGAACCTCC ATCCCACTCT TGTTATGACT |
| 1561 | CCACAGTGTC CAGACTAATT TGTGCATGAA CTGAAATAAA |
| 1601 | ACCATCCTAC GGTATCCAGG GAACAGAAAG CAGGATGCAG |
| 1641 | GATGGGAGGA CAGGAAGGCA GCCTGGGACA TTTAAAAAAA |
| 1681 | TAAAAATGAA AAAAAAACCC AGAACCCATT TCTCAGGGCA |
| 1721 | CTTTCCAGAA TTCTCTCATA TTTGTGGGCT GGGATCAAGC |
| 1761 | CTGCAGCTTG AGGAAAGCAC AAGGAAAGGA AAGAAGATCT |
| 1801 | GGTGGAAAGC TCAGGTGGCA GCGGACTCTG ACTCCACTGA |
| 1841 | GGAACTGCCT CAGAAGCTGC GATCACAACT TTGGCTGAAG |
| 1881 | CCCCTGCCTC ACTCTAGGGC ACCTGACCTG GCCTCTTGCC |
| 1921 | TAAACCACAA GGCTAAGGGC TATAGACAAT GGTTTCCTTA |
| 1961 | GGAACAGTAA ACCAGTTTTT CTAGGGATGG CCCTTGGCTG |
| 2001 | GGGGATGACA GTGTGGGAGC TGTGGGGTAC TGAGGAAGAC |
| 2041 | ACCATTCCTT GACGGTGTCT AAGAAGCCAG GTGGATGTGT |
| 2081 | GTGGTGGCTC CAGTGGGTGT TTCTACTCTG CCAGTGAGAG |
| 2121 | GCAGCCCCCT AGAAACTCTT CAGGCGTAAT GGAAAATCAG |
| 2161 | CTCAAATGAG ATCAGGCCCC CCCAGGGTCC ACCCACAGAG |
| 2201 | CACTACAGAG CCTCTGAAAG ACCATAGCAC CAAGCGAGCC |
| 2241 | CCTTCAGATT CCCCCACTGT CCATCGGAAG ATGCTCCAGA |
| 2281 | GTGGCTAGAG GGCATCTAAG GGCTCCAGCA TGGCATATCC |
| 2321 | ATGCCCACGG TGCTGTGTCC ATGATCTGAG TGATAGCTGC |
| 2361 | ACTGCTGCCT GGGATTGCAG CTGAGGTGGG AGTGGAGAAT |
| 2401 | GGTTCCCAGG AAGACAGTTC CACCTCTAAG GTCCGAAAAT |
| 2441 | GTTCCCTTTA CCCTGGAGTG GGAGTGAGGG GTCATACACC |
| 2481 | AAAGGTATTT TCCCTCACCA GTCTAGGCAT GACTGGCTTC |
| 2521 | TGAAAAATTC CAGCACACCT CCTCGAACCT CATTGTCAGC |
| 2561 | AGAGAGGGCC CATCTGTTGT CTGTAACATG CCTTTCACAT |
| 2601 | GTCCACCTTC TTGCCATGTT CCAGCTGCTC TCCCAACCTG |
| 2641 | GAAGGCCGTC TCCCCTTAGC CAAGTCCTCC TCAGGCTTGG |
| 2681 | AGAACTTCCT CAGCGTCACC TCCTTCATTG AGCCTTCTCT |
| 2721 | GATCACTCCA TCCCTCTCCT ACCCCTCCCT CCCCCAACCC |
| 2761 | TCAATGTATA AATTGCTTCT TGATGCTTAG CATTCACAAT |
| 2801 | TTTTGATTGA TCGTTATTTG TGTGTGTGTG TCCGATCTCA |
| 2841 | CAAGTATATT GTAAACCCTT CGGTGGGTGG GGGCCATATC |
| 2881 | CTAGACCTCT CTGTATCCCC CAGACTATCT GTAACAGTGC |
| 2921 | CAGGCACACA GTAGGTGATC AATAAACACT TGTTGATTGA |
| 2961 | G |

An amino acid sequence for a human claudin-5 (CLDN5) isoform 2 polypeptide is available in the NCBI and UNIPROT databases (NCBI accession no. NP_001349995; see also UNIPROT accession no. 000501.1) and shown below as SEQ ID NO:11.

| | |
|---|---|
| 1 | MGSAALEILG LVLCLVGWGG LILACGLPMW QVTAFLDHNI |
| 41 | VIAQTTWKGL WMSCVVQSTG HMQCKVYDSV LALSTEVQAA |
| 81 | RALTVSAVLL AFVALFVTLA GAQCTTCVAP GPAKARVALT |
| 121 | GGVLYLFCGL LALVPLCWFA NIVVREFYDP SVPVSQKYEL |
| 161 | GAALYIGWAA TALLMVGGCL LCCGAWVCTG RPDLSFPVKY |
| 201 | SAPRRPTATG DYDKKNYV |

The CLDN5 gene encodes the CLDN5 polypeptide with SEQ ID NO:11. The CLDN5 gene is on chromosome 22 (location NC_000022.11 (19523024 . . . 19525337, complement)). A cDNA sequence that encodes the CLDN5 polypeptide with SEQ ID NO: 11 is available as NCBI accession no. NM_001363066, shown below as SEQ ID NO: 12.

| | |
|---|---|
| 1 | GGCAGACCCA GGAGGTGCGA CAGACCCGCG GGGCAAACGG |
| 41 | ACTGGGGCCA AGAGCCGGGA GCGCGGGCGC AAAGGCACCA |
| 81 | GGGCCCGCCC AGGGCGCCGC GCAGCACGGC CTTGGGGGTT |
| 121 | CTGCGGGCCT TCGGGTGCGC GTCTCGCCTC TAGCCATGGG |
| 161 | GTCCGCAGCG TTGGAGATCC TGGGCCTGGT GCTGTGCCTG |
| 201 | GTGGGCTGGG GGGGTCTGAT CCTGGCGTGC GGGCTGCCCA |
| 241 | TGTGGCAGGT GACCGCCTTC CTGGACCACA ACATCGTGAC |
| 281 | GGCGCAGACC ACCTGGAAGG GGCTGTGGAT GTCGTGCGTG |
| 321 | GTGCAGAGCA CCGGGCACAT GCAGTGCAAA GTGTACGACT |
| 361 | CGGTGCTGGC TCTGAGCACC GAGGTGCAGG CGGCGCGGGC |
| 401 | GCTCACCGTG AGCGCCGTGC TGCTGGCGTT CGTTGCGCTC |
| 441 | TTCGTGACCC TGGGGGGCGC GCAGTGCACC ACCTGCGTGG |
| 481 | CCCCGGGCCC GGCCAAGGCG CGTGTGGCCC TCACGGGAGG |
| 521 | CGTGCTCTAC CTGTTTTGCG GGCTGCTGGC GCTCGTGCCA |
| 561 | CTCTGCTGGT TCGCCAACAT TGTCGTCCGC GAGTTTTACG |
| 601 | ACCCGTCTGT GCCCGTGTCG CAGAAGTACG AGCTGGGCGC |
| 641 | AGCGCTGTAC ATCGGCTGGG CGGCCACCGC GCTGCTCATG |
| 681 | GTAGGCGGCT GCCTCTTGTG CTGCGGCGCC TGGGTCTGCA |
| 721 | CCGGCCGTCC CGACCTCAGC TTCCCCGTGA AGTACTCAGC |
| 761 | GCCGCGGCGG CCCACGGCCA CCGGCGACTA CGACAAGAAG |
| 801 | AACTACGTCT GAGGGCGCTG GGCACGGCCG GGCCCCTCCT |
| 841 | GCCAGCCACG CCTGCGAGGC GTTGGATAAG CCTGGGGAGC |
| 881 | CCCGCATGGA CCGCGGCTTC CGCCGGGTAG CGCGGCGCGC |
| 921 | AGGCTCCTCG GAACGTCCGG CTCTGCGCCC CGACGCGGCT |
| 961 | CCTGGATCCG CTCCTGCCTG CGCCCGCAGC TGACCTTCTC |
| 1001 | CTGCCACTAG CCCGGCCCTG CCCTTAACAG ACGGAATGAA |
| 1041 | GTTTCCTTTT CTGTGCGCGG CGCTGTTTCC ATAGGCAGAG |
| 1081 | CGGGTGTCAG ACTGAGGATT TCGCTTCCCC TCCAAGACGC |
| 1121 | TGGGGGTCTT GGCTGCTGCC TTACTTCCCA GAGGCTCCTG |
| 1161 | CTGACTTCGG AGGGGCGGAT GCAGAGCCCA GGGCCCCCAC |
| 1201 | CGGAAGATGT GTACAGCTGG TCTTTACTCC ATCGGCAGGG |
| 1241 | CCCGAGCCCA GGGACCAGTG ACTTGGCCTG GACCTCCCGG |
| 1281 | TCTCACTCCA GCATCTCCCC AGGCAAGGCT TGTGGGCACC |
| 1321 | GGAGCTTGAG AGAGGGCGGG AGTGGGAAGG CTAAGAATCT |
| 1361 | GCTTAGTAAA TGGTTTGAAC TCTC |

An amino acid sequence for a human claudin-6 (CLDN6) polypeptide is available in the NCBI and UNIPROT databases (NCBI accession no. NP_067018; see also UNIPROT accession no. P56747.2) and shown below as SEQ ID NO:13.

| | |
|---|---|
| 1 | MASAGMQILG VVLTLLGWVN GLVSCALPMW KVTAFIGNSI |
| 41 | VVAQVVWEGL WMSCVVQSTG QMQCKVYDSL LALPQDLQAA |
| 81 | RALCVIALLV ALFGLIVYLA GAKCTTCVEE KDSKARLVLT |
| 121 | SGIVFVISGV LTLIPVCWTA HAIIRDFYNP LVAEAQKREL |
| 161 | GASLYLGWAA SGLLLLGGGL LCCTCPSGGS QGPSHYMARY |
| 181 | STSAPAISRG PSEYPTKNYV |

The CLDN6 gene encodes the CLDN6 polypeptide with SEQ ID NO:13. The CLDN6 gene is on chromosome 16 (location NC_000016.10 (3014712 . . . 3018183, complement)). A cDNA sequence that encodes the CLDN6 polypeptide with SEQ ID NO: 13 is available as NCBI accession no. NM_021195.5, shown below as SEQ ID NO: 14.

| | |
|---|---|
| 1 | ACTCGGCCTA GGAATTTCCC TTATCTCCTT CGCAGTGCAG |
| 41 | CTCCTTCAAC CTCGCCATGG CCTCTGCCGG AATGCAGATC |
| 81 | CTGGGAGTCG TCCTGACACT GCTGGGCTGG GTGAATGGCC |
| 121 | TGGTCTCCTG TGCCCTGCCC ATGTGGAAGG TGACCGCTTT |
| 161 | CATCGGCAAC AGCATCGTGG TGGCCCAGGT GGTGTGGGAG |
| 201 | GGCCTGTGGA TGTCCTGCGT GGTGCAGAGC ACCGGCCAGA |
| 241 | TGCAGTGCAA GGTGTACGAC TCACTGCTGG CGCTGCCACA |
| 281 | GGACCTGCAG GCTGCACGTG CCCTCTGTGT CATCGCCCTC |
| 321 | CTTGTGGCCC TGTTCGGCTT GCTGGTCTAC CTTGCTGGGG |
| 361 | CCAAGTGTAC CACCTGTGTG GAGGAGAAGG ATTCCAAGGC |
| 401 | CCGCCTGGTG CTCACCTCTG GGATTGTCTT TGTCATCTCA |
| 441 | GGGGTCCTGA CGCTAATCCC CGTGTGCTGG ACGGCGCATG |
| 481 | CCATCATCCG GGACTTCTAT AACCCCCTGG TGGCTGAGGC |
| 521 | CCAAAAGCGG GAGCTGGGGG CCTCCCTCTA CTTGGGCTGG |
| 561 | GCGGCCTCAG GCCTTTTGTT GCTGGGTGGG GGGTTGCTGT |
| 601 | GCTGCACTTG CCCCTCGGGG GGGTCCCAGG GCCCCAGCCA |
| 641 | TTACATGGCC CGCTACTCAA CATCTGCCCC TGCCATCTCT |
| 681 | CGGGGGCCCT CTGAGTACCC TACCAAGAAT TACGTCTGAC |
| 721 | GTGGAGGGGA ATGGGGGCTC CGCTGGCGCT AGAGCCATCC |
| 761 | AGAAGTGGCA GTGCCCAACA GCTTTGGGAT GGGTTCGTAC |
| 801 | CTTTTGTTTC TGCCTCCTGC TATTTTTCTT TTGACTGAGG |
| 841 | ATATTTAAAA TTCATTTGAA AACTGAGCCA AGGTGTTGAC |
| 881 | TCAGACTCTC ACTTAGGCTC TGCTGTTTCT CACCCTTGGA |
| 921 | TGATGGAGCC AAAGAGGGGA TGCTTTGAGA TTCTGGATCT |
| 961 | TGACATGCCC ATCTTAGAAG CCAGTCAAGC TATGGAACTA |
| 1001 | ATGCGGAGGC TGCTTGCTGT GCTGGCTTTG CAACAAGACA |
| 1041 | GACTGTCCCC AAGAGTTCCT GCTGCTGCTG GGGGCTGGGC |
| 1081 | TTCCCTAGAT GTCACTGGAC AGCTGCCCCC CATCCTACTC |
| 1121 | AGGTCTCTGG AGCTCCTCTC TTCACCCCTG GAAAAACAAA |
| 1161 | TGATCTGTTA ACAAAGGACT GCCCACCTCC GGAACTTCTG |
| 1201 | ACCTCTGTTT CCTCCGTCCT GATAAGACGT CCACCCCCCA |
| 1241 | GGGCCAGGTC CCAGCTATGT AGACCCCCGC CCCCACCTCC |
| 1281 | AACACTGCAC CCTTCTGCCC TGCCCCCCTC GTCTCACCCC |
| 1321 | CTTTACACTC ACATTTTTAT CAAATAAAGC ATGTTTTGTT |
| 1361 | AGTGCA |

An amino acid sequence for a human claudin-7 (CLDN7) isoform 1 polypeptide is available in the NCBI and UNIPROT databases (NCBI accession no. NP_001298; see also UNIPROT accession no. 095471.4) and shown below as SEQ ID NO: 15.

| | |
|---|---|
| 1 | MANSGLQLLG FSMALLGWVG LVACTAIPQW QMSSYAGDNI |
| 41 | ITAQAMYKGL WMDCVTQSTG MMSCKMYDSV LALSAALQAT |
| 81 | RALMVVSLVL GFLAMFVATM GMKCTRCGGD DKVKKARIAM |
| 121 | GGGIIFIVAG LAALVACSWY GHQIVTDFYN PLIPTNIKYE |
| 161 | FGPAIFIGWA GSALVILGGA LLSCSCPGNE SKAGYRVPRS |
| 201 | YPKSNSSKEY V |

The CLDN7 gene encodes the CLDN7 polypeptide with SEQ ID NO:15. The CLDN7 gene is on chromosome 17 (NC_000017.11 (7259903 . . . 7263213, complement). A cDNA sequence that encodes the CLDN7 polypeptide with SEQ ID NO: 15 is available as NCBI accession no. NM_001307.6, shown below as SEQ ID NO:16.

| | |
|---|---|
| 1 | GCCCGCACCT GCTGGCTCAC CTCCGAGCCA CCTCTGCTGC |
| 41 | GCACCGCAGC CTCGGACCTA CAGCCCAGGA TACTTTGGGA |
| 81 | CTTGCCGGCG CTCAGAAACG CGCCCAGACG GCCCCTCCAC |
| 121 | CTTTTGTTTG CCTAGGGTCG CCGAGAGCGC CCGGAGGGAA |
| 161 | CCGCCTGGCC TTCGGGGACC ACCAATTTTG TCTGGAACCA |
| 201 | CCCTCCCGGC GTATCCTACT CCCTGTGCCG CGAGGCCATC |
| 241 | GCTTCACTGG AGGGGTCGAT TTGTGTGTAG TTTGGTGACA |
| 281 | AGATTTGCAT TCACCTGGCC CAAACCCTTT TTGTCTCTTT |
| 321 | GGGTGACCGG AAAACTCCAC CTCAAGTTTT CTTTTGTGGG |
| 361 | GCTGCCCCCC AAGTGTCGTT TGTTTTACTG TAGGGTCTCC |
| 401 | CCGCCCGGCG CCCCCAGTGT TTTCTGAGGG CGGAAATGGC |
| 441 | CAATTCGGGC CTGCAGTTGC TGGGCTTCTC CATGGCCCTG |
| 481 | CTGGGCTGGG TGGGTCTGGT GGCCTGCACC GCCATCCCGC |
| 521 | AGTGGCAGAT GAGCTCCTAT GCGGGTGACA ACATCATCAC |
| 561 | GGCCCAGGCC ATGTACAAGG GGCTGTGGAT GGACTGCGTC |
| 601 | ACGCAGAGCA CGGGGATGAT GAGCTGCAAA ATGTACGACT |
| 641 | CGGTGCTCGC CCTGTCCGCG GCCTTGCAGG CCACTCGAGC |
| 681 | CCTAATGGTG GTCTCCCTGG TGCTGGGCTT CCTGGCCATG |
| 721 | TTTGTGGCCA CGATGGGCAT GAAGTGCACG CGCTGTGGGG |
| 761 | GAGACGACAA AGTGAAGAAG GCCCGTATAG CCATGGGTGG |
| 801 | AGGCATAATT TTCATCGTGG CAGGTCTTGC CGCCTTGGTA |
| 841 | GCTTGCTCCT GGTATGGCCA TCAGATTGTC ACAGACTTTT |
| 881 | ATAACCCTTT GATCCCTACC AACATTAAGT ATGAGTTTGG |
| 921 | CCCTGCCATC TTTATTGGCT GGGCAGGGTC TGCCCTAGTC |
| 961 | ATCCTGGGAG GTGCACTGCT CTCCTGTTCC TGTCCTGGGA |
| 1001 | ATGAGAGCAA GGCTGGGTAC CGTGTACCCC GCTCTTACCC |
| 1041 | TAAGTCCAAC TCTTCCAAGG AGTATGTGTG ACCTGGGATC |
| 1081 | TCCTTGCCCC AGCCTGACAG GCTATGGGAG TGTCTAGATG |
| 1121 | CCTGAAAGGG CCTGGGGCTG AGCTCAGCCT GTGGGCAGGG |
| 1161 | TGCCGGACAA AGGCCTCCTG GTCACTCTGT CCCTGCACTC |
| 1201 | CATGTATAGT CCTCTTGGGT TGGGGGTGGG GGGGTGCCGT |
| 1241 | TGGTGGGAGA GACAAAAAGA GGGAGAGTGT GCTTTTTGTA |
| 1281 | CAGTAATAAA AAATAAGTAT TGGGAAGCAG GCTTTTTTCC |
| 1321 | CTTCAGGGCC TCTGCTTTCC TCCCGTCCAG ATCCTTGCAG |
| 1361 | GGAGCTTGGA ACCTTAGTGC ACCTACTTCA GTTCAGAACA |
| 1401 | CTTAGCACCC CACTGACTCC ACTGACAATT GACTAAAAGA |
| 1441 | TGCAGGTGCT CGTATCTCGA CATTCATTCC CACCCCCCTC |
| 1481 | TTATTTAAAT AGCTACCAAA GTACTTCTTT TTTAATAAAA |
| 1521 | AAATAAAGAT TTTTATTAGG TA |

### Variants and Modified Tight Junction Proteins

Zonula occludens, OCLN, and claudin (CLDN) sequences can vary amongst the human population. Variants can include codon variations and/or conservative amino acid changes. Zonula occludens (TJP), OCLN, and claudin (CLDN) nucleotide and protein sequences can also include non-conservative variations. For example, the zonula occludens (TJP), OCLN, and claudin (CLDN) nucleic acids or proteins can have at least 85% sequence identity and/or complementary, or at least 90% sequence identity and/or complementary, or at least 95% sequence identity and/or complementarity, or at least 96% sequence identity and/or complementarity, or at least 97% sequence identity and/or complementarity, or at least 98% sequence identity and/or complementarity, or at least 99% sequence identity and/or complementarity to any of the Zonula occludens (TJP), OCLN, and claudin (CLDN) nucleic acid or protein sequences described herein.

As illustrated herein, inhibition or loss of function of tight junction gene products (e.g., ZO1) can facilitate conversion of hiPSCs to primordial germ cells. Loss of function modifications to tight junction genes and gene products can be introduced by any method. Other possible methods of silencing/disrupting tight junction genes include using short interfering RNA (siRNA), using CRISPR to knockout or mutate a tight junction gene, or simply using chemical inhibition (EDTA or other calcium chelators, for example).

For example, genetic loci encoding tight junction proteins can be modified in human iPSC lines by deletion, insertion, or substitution. A variety of methods and inhibitors can be used to reduce the function of these tight junction proteins. For example, the hiPSCs or iMeLCs can be contacted with CRISPRi ribonucleoprotein (RNP) complexes, inhibitory nucleic acids, expression vectors, virus-like particles (VLP), CRISPR-related, and combinations thereof that target the tight junction genes or mRNAs.

The CRISPR-Cas9 genome-editing system can be used to delete modify tight junction coding regions or regulatory elements. A single guide RNA (sgRNA) can be used to recognize one or more target sequence in a subject's genome, and a nuclease can act as a pair of scissors to cleave a single-strand or a double-strand of genomic DNA. Mutations in the genome that are near the cleavage site can be introduced by an endogenous Non-Homologous End Joining (NHEJ) or Homology Directed Repair (HDR) pathway. Hence, the guide RNAs guide the nuclease to cleave the targeted tight junction genomic site for deletion and/or modification by endogenous mechanisms.

The Cas system can recognize any sequence in the genome that matches 20 bases of a gRNA. However, each gRNA should also be adjacent to a “Protospacer Adjacent Motif” (PAM), which is invariant for each type of Cas protein, because the PAM binds directly to the Cas protein. See Doudna et al., Science 346 (6213): 1077, 1258096 (2014); and Jinek et al., Science 337:816-21 (2012). Hence, the guide RNAs can have a PAM site sequence that can be bound by a Cas protein.

When the Cas system was first described for Cas9, with a “NGG” PAM site, the PAM was somewhat limiting in that it required a GG in the right orientation to the site to be targeted. Different Cas9 species have now been described with different PAM sites. See Jinek et al., Science 337:816-21 (2012); Ran et al., Nature 520:186-91 (2015); and Zetsche et al., Cell 163:759-71 (2015). In addition, mutations in the PAM recognition domain (Table 1) have increased the diversity of PAM sites for SpCas9 and SaCas9. See Kleinstiver et al., Nat Biotechnol 33:1293-1298 (2015); and Kleinstiver et al., Nature 523:481-5 (2015). The following are examples of PAM sites.

TABLE 1
PAM Sequences

| Cas Nuclease | PAM Sequence |
|---|---|
| SpCas9 | NGG |
| SpCas9 VRER variant | NGCG |
| SpCas9 EQR variant | NGAG |
| SpCas9 VQR variant | NGAN or NGNG |
| SaCas9 | NNGRRT |
| SaCas9, KKH variant | NNNRRT |
| FnCas2 (Cpf1) | TTN |

DNA annotations:
N = A, C, T or G;
R = Purine, A or G
Note that the guide RNAs for SpCas9 and SaCas9 cover 20 bases in the 5′direction of the PAM site, while for FnCas2 (Cpf1) the guide RNA covers 20 bases to 3′ of the PAM.

There are a number of different types of nucleases and systems that can be used for gene editing. The nuclease employed can in some cases be any DNA binding protein with nuclease activity. Examples of nuclease include Streptococcus pyogenes Cas (SpCas9) nucleases, Staphylococcus aureus Cas9 (SpCas9) nucleases, Francisella novicida Cas2 (FnCas2, also called dFnCpf1) nucleases, Zinc Finger Nucleases (ZFN), Meganuclease, Transcription activator-like effector nucleases (TALEN), Fok-I nucleases, any DNA binding protein with nuclease activity, any DNA binding protein bound to a nuclease, or any combinations thereof. However, the CRISPR-Cas systems are generally the most widely used. In some cases, the nuclease is therefore a Cas nuclease.

CRISPR-Cas systems are generally divided into two classes. The class 1 system contains types I, III and IV, and the class 2 system contains types II, V, and VI. The class 1 CRISPR-Cas system uses a complex of several Cas proteins, whereas the class 2 system only uses a single Cas protein with multiple domains. The class 2 CRISPR-Cas system is usually preferable for gene-engineering applications because of its simplicity and ease of use.

A variety of Cas nucleases can be employed in the methods described herein. Three species that have been best characterized are provided as examples. The most commonly used Cas nuclease is a Streptococcus pyogenes Cas9, (SpCas9). More recently described forms of Cas include Staphylococcus aureus Cas9 (SaCas9) and Francisella novicida Cas2 (FnCas2, also called FnCpf1). Jinek et al., Science 337:816-21 (2012); Qi et al., Cell 152:1173-83 (2013); Ran et al., Nature 520:186-91 (2015); Zetsche et al., Cell 163:759-71 (2015).

Inhibitory nucleic acids can be used to reduce the expression and/or translation of tight junction. Such inhibitory nucleic acids can specifically bind to tight junction nucleic acids, including nascent RNAs, that encode a tight junction protein. Anti-sense oligonucleotides have been used to silence regulatory elements as well.

An inhibitory nucleic acid can have at least one segment that will hybridize to tight junction nucleic acid under intracellular or stringent conditions. The inhibitory nucleic acid can reduce processing, expression, and/or translation of a nucleic acid encoding tight junction. An inhibitory nucleic acid may hybridize to a genomic DNA, a messenger RNA, nascent RNA, or a combination thereof. An inhibitory nucleic acid may be incorporated into a plasmid vector or viral DNA. It may be single stranded or double stranded, circular, or linear.

An inhibitory nucleic acid can be a polymer of ribose nucleotides (RNAi) or deoxyribose nucleotides having more than 13 nucleotides in length. An inhibitory nucleic acid may include naturally-occurring nucleotides; synthetic, modified, or pseudo-nucleotides such as phosphorothiolates; as well as nucleotides having a detectable label such as P³², biotin or digoxigenin. An inhibitory nucleic acid can reduce the expression, processing, and/or translation of a tight junction nucleic acid.

Such an inhibitory nucleic acid may be completely complementary to a segment of tight junction nucleic acid (e.g., a tight junction mRNA or tight junction nascent transcript).

An inhibitory nucleic acid can hybridize to a tight junction nucleic acid under intracellular conditions or under stringent hybridization conditions and is sufficient to inhibit expression of a tight junction nucleic acid. Intracellular conditions refer to conditions such as temperature, pH and salt concentrations typically found inside a cell, e.g. a target cell described herein.

Generally, stringent hybridization conditions are selected to be about 5° C. lower than the thermal melting point (Tm) for the specific sequence at a defined ionic strength and pH. However, stringent conditions encompass temperatures in the range of about 1° C. to about 20° C. lower than the thermal melting point of the selected sequence, depending upon the desired degree of stringency as otherwise qualified herein. Inhibitory oligonucleotides that comprise, for example, 2, 3, 4, or 5 or more stretches of contiguous nucleotides that are precisely complementary to a tight junction coding or flanking sequence, can each be separated by a stretch of contiguous nucleotides that are not complementary to adjacent coding sequences, and such an inhibitory nucleic acid can still inhibit the function of a tight junction nucleic acid. In general, each stretch of contiguous nucleotides is at least 4, 5, 6, 7, or 8 or more nucleotides in length. Non-complementary intervening sequences may be 1, 2, 3, or 4 nucleotides in length.

One skilled in the art can easily use the calculated melting point of an inhibitory nucleic acid hybridized to a sense nucleic acid to estimate the degree of mismatching that will be tolerated for inhibiting expression of a particular target nucleic acid. Inhibitory nucleic acids of the invention include, for example, a short hairpin RNA, a small interfering RNA, a ribozyme, or an antisense nucleic acid molecule.

The inhibitory nucleic acid molecule may be single (e.g., an antisense oligonucleotide) or double stranded (e.g., a siRNA) and may function in an enzyme-dependent manner or by steric blocking. Inhibitory nucleic acid molecules that function in an enzyme-dependent manner include forms dependent on RNase H activity to degrade target mRNA. These include single-stranded DNA, RNA, and phosphorothioate molecules, as well as the double-stranded RNAi/siRNA system that involves target mRNA recognition through sense-antisense strand pairing followed by degradation of the target mRNA by the RNA-induced silencing complex. Steric blocking inhibitory nucleic acids, which are RNase-H independent, interfere with gene expression or other mRNA-dependent cellular processes by binding to a target mRNA and getting in the way of other processes. Steric blocking inhibitory nucleic acids include 2′-O alkyl (usually in chimeras with RNase-H dependent antisense), peptide nucleic acid (PNA), locked nucleic acid (LNA) and morpholino antisense.

Small interfering RNAs (siRNAs), for example, may be used to specifically reduce tight junction processing or translation such that production of the encoded polypeptide is reduced. SiRNAs mediate post-transcriptional gene silencing in a sequence-specific manner. See, for example, website at invitrogen.com/site/us/en/home/Products-and-Services/Applications/mai.html. Once incorporated into an RNA-induced silencing complex, siRNA can mediate cleavage of the homologous endogenous mRNA transcript by guiding the complex to the homologous mRNA transcript, which is then cleaved by the complex. The siRNA may be homologous to any region of the tight junction mRNA transcript. The region of homology may be 50 nucleotides or less, 30 nucleotides or less in length, such as less than 25 nucleotides, or for example about 21 to 23 nucleotides in length. SiRNA is typically double stranded and may have two-nucleotide 3′ overhangs, for example, 3′ overhanging UU dinucleotides. Methods for designing siRNAs are available, see, for example, Elbashir et al. Nature 411:494-498 (2001); Harborth et al. Antisense Nucleic Acid Drug Dev. 13:83-106 (2003).

The pSuppressorNeo vector for expressing hairpin siRNA, commercially available from IMGENEX (San Diego, California), can be used to make siRNA or shRNA for inhibiting tight junction expression. The construction of the siRNA or shRNA expression plasmid involves the selection of the target region of the mRNA, which can be a trial-and-error process. However, Elbashir et al. have provided guidelines that appear to work ˜80% of the time. Elbashir, S. M., et al., Analysis of gene function in somatic mammalian cells using small interfering RNAs. Methods, 2002. 26 (2): p. 199-213. Accordingly, for synthesis of synthetic siRNA or shRNA, a target region may be selected preferably 50 to 100 nucleotides downstream of the start codon. The 5′ and 3′ untranslated regions and regions close to the start codon should be avoided as these may be richer in regulatory protein binding sites. As siRNA can begin with AA, have 3′ UU overhangs for both the sense and antisense siRNA strands, and have an approximate 50% G/C content. An example of a sequence for a synthetic siRNA or shRNA is 5′-AA (N19) UU, where N is any nucleotide in the mRNA sequence and should be approximately 50% G-C content. The selected sequence(s) can be compared to others in the human genome database to minimize homology to other known coding sequences (e.g., by Blast search, for example, through the NCBI website).

Inhibitory nucleic acids (e.g., siRNAs, and/or anti-sense oligonucleotides) may be chemically synthesized, created by in vitro transcription, or expressed from an expression vector or a PCR expression cassette. See, e.g., website at invitrogen.com/site/us/en/home/Products-and-Services/Applications/rai.html.

When an siRNA is expressed from an expression vector or a PCR expression cassette, the insert encoding the siRNA may be expressed as an RNA transcript that folds into an siRNA hairpin or a shRNA. Thus, the RNA transcript may include a sense siRNA sequence that is linked to its reverse complementary antisense siRNA sequence by a spacer sequence that forms the loop of the hairpin as well as a string of U's at the 3′ end. The loop of the hairpin may be of any appropriate lengths, for example, 3 to 30 nucleotides in length, or about 3 to 23 nucleotides in length, and may include various nucleotide sequences including for example, AUG, CCC, UUCG, CCACC, CTCGAG, AAGCUU, and CCACACC. SiRNAs also may be produced in vivo by cleavage of double-stranded RNA introduced directly or via a transgene or virus. Amplification by an RNA-dependent RNA polymerase may occur in some organisms.

An inhibitory nucleic acid such as a short hairpin RNA siRNA or an antisense oligonucleotide may be prepared using methods such as by expression from an expression vector or expression cassette that includes the sequence of the inhibitory nucleic acid. Alternatively, it may be prepared by chemical synthesis using naturally-occurring nucleotides, modified nucleotides, or any combinations thereof. In some embodiments, the inhibitory nucleic acids are made from modified nucleotides or non-phosphodiester bonds, for example, that are designed to increase biological stability of the inhibitory nucleic acid or to increase intracellular stability of the duplex formed between the inhibitory nucleic acid and the target tight junction nucleic acid.

### Differentiation of Primordial Germ Cells

Primordial germ cells can be differentiated into mature germ cells, including functional oocyte and sperm by in vitro culture or by implantation in a selected subject. A variety of differentiation methods can be used including those described in U.S. patent application No. 20180251729. Previous studies in mice illustrate methods for generating functional male and female gametes from PGCLCs in vivo, which can then be used to produce live offspring through IVF (Hayashi et al., Cell 2011) (Hayashi et al., Science 2013) (Zhou et al., Science 2013). Xenogenic and allogenic transplantation of primordial germ cells into the ovarian bursa, seminiferous tubules of the testes, or under the kidney capsule of mice successfully induced meiosis in the transplanted PGCs, establishing a proof-of-concept method for PGC maturation that potentially circumvents the need for developing an in vitro protocol to mature human PGCs (Hayama et al., Biol. Reprod 2014) (Matoba et al., Biol. Reprod 2011) (Qing et al., Hum. Reprod. 2008). Additionally, it has recently been shown that human female PGCs can be matured to oogonia by xenogeneic culture with mouse embryonic ovarian somatic cells (Yamashiro et al., Science 2020).

The following Examples illustrate some of the experiments that were performed in the development of the invention.

### Example 1: Methods

This Example describes some of the materials and methods used in developing the invention.

### Cell Culture

Human iPSC lines were derived from the male Allen Institute WTC-LMNB1-meGFP line (Cell Line ID: AICS-0013 cl.210, passage 32) obtained from Coriel, and/or the female WTB CRISPRi-Gen1B line (Gladstone Stem Cell Core, passage 40) provided by Dr. Bruce Conklin's lab. For routine culture, human induced pluripotent stem cells (hiPSCs) were grown feeder-free on growth factor reduced Matrigel (BD Biosciences) and fed daily with mTESR1 medium (Stem Cell Technologies). Cells were passaged every 3-4 days with Accutase (Stem Cell Technologies) and seeded at a density of 12,000 cells/cm2. ROCK inhibitor Y-276932 (10 uM; Selleckchem) was added to the media to promote cell survival after passaging. All generated cell lines were karyotyped prior to expansion and confirmed as normal cells both by Cell Line Genetics and by using the hPSC Genetic Analysis Kit (Stem Cell Technologies Cat. #07550). The cells were also regularly tested for mycoplasma using a MycoAlert Mycoplasma Detection Kit (Lonza).

### Generation of CRISPi Lines

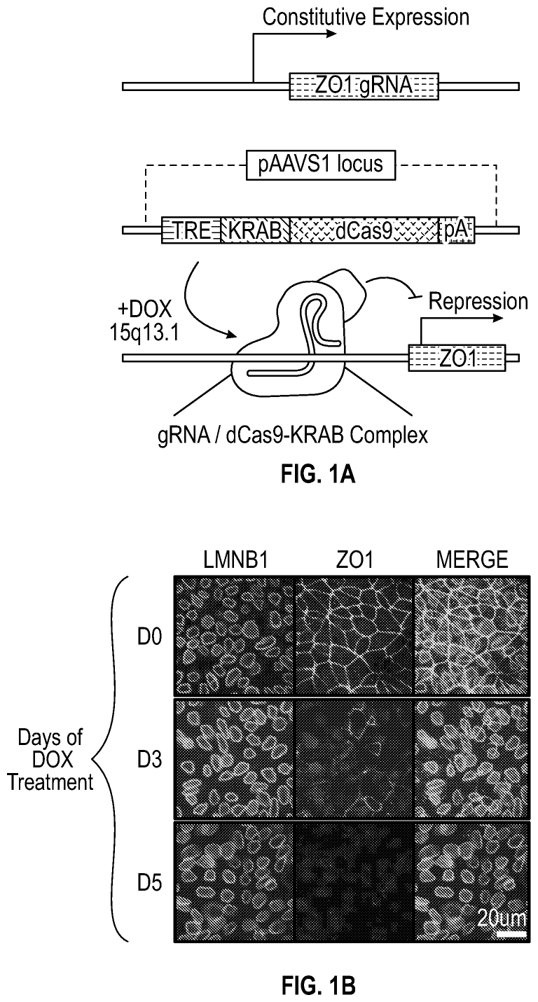

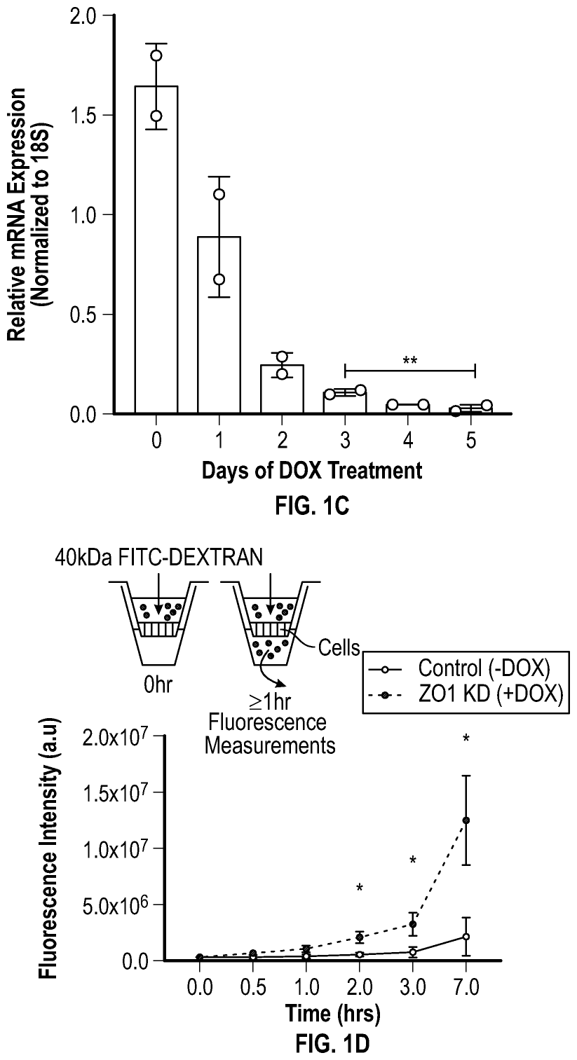

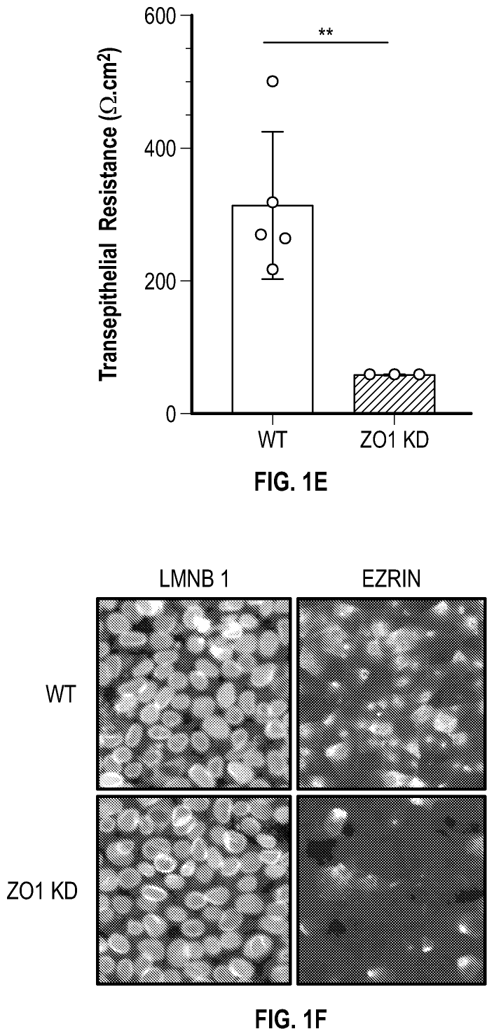

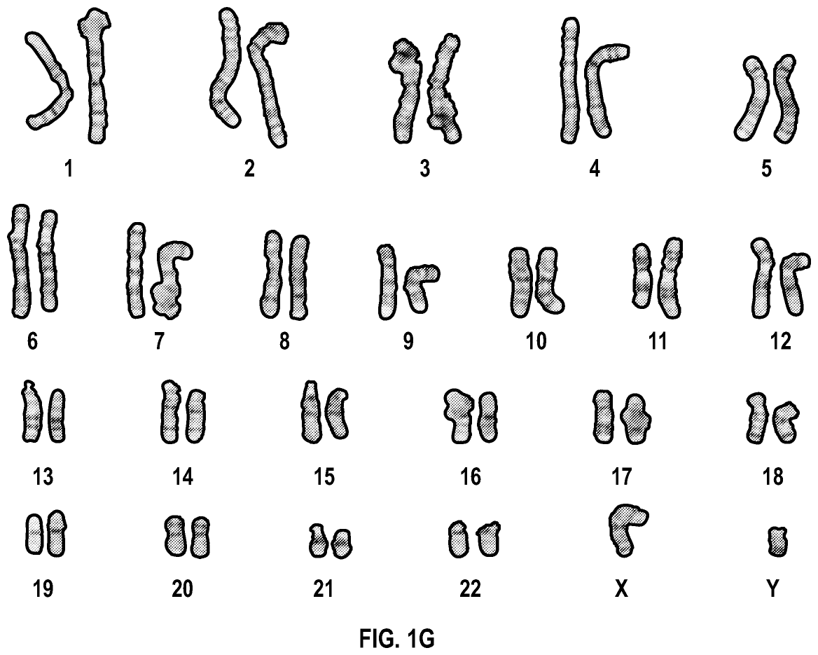

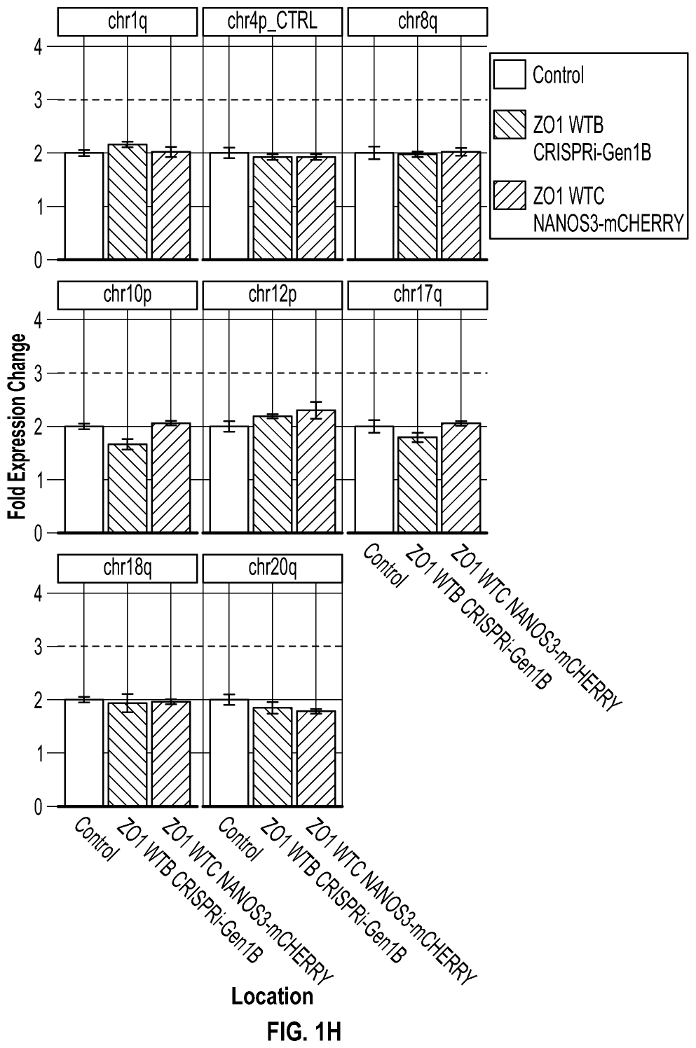

Knockdown (KD) of ZO1 in hiPSC lines was achieved using a doxycycline (DOX) inducible CRISPR interference (CRISPRi) system, which included two components. First, a dCas9-KRAB repressor driven by a Tet-on-3G promoter was knocked in into the AAVS1 safe harbor locus and expressed only under DOX treatment described by Mandegar et al. Cell Stem Cell 18, 541-553 (2016) (FIG. 1A). Second, a constitutively expressed guide RNA (gRNA) was used that targets the transcriptional start site of a gene (FIG. 1A). Briefly, about 2 million WTC or WTB derived cells were nucleofected with the knockin vector (5 ug) along with TALENS targeting the AAVS1 locus (2 ug) and cultured in mTESR1 and ROCK inhibitor Y-276932 (10 uM). Knockin selection was performed with Genticin (100 ug/mL Life Technologies) over the course of 10 days, and a clonal population was generated through colony picking under the EVOS picking microscope (Life Technologies).

To generate the ZO1-WTC line, four CRISPRi gRNAs were designed to bind within 150 bp of the transcription start site of ZO1 and cloned into the gRNA-CKB vector at the BsmB1 restriction site, following the protocol described in Mandegar et al. (2016). The sequences of the ZO1 guide RNAs that were used are shown in Table 2 below.

TABLE 2
CRISPRi gRNAs

| Guide RNA | Location | |
|---|---|---|
| (gRNA) Target | to TSS | Sequence |
| ZO1_1 | 67 | CCGGTTCCCGGGAAGTTACG |
| | | (SEQ ID NO: 17) |
| ZO1_2 | 271 | CAGGGGGAGGGAATTCAACT |
| | | (SEQ ID NO: 18) |
| ZO1_3 | 147 | CTTTCGCAGCCCGGCCACGT |
| | | (SEQ ID NO: 19) |
| ZO1_4 | 76 | GGGAAGTTACGTGGCGAAGC |
| | | (SEQ ID NO: 20) |

Vectors containing each gRNA sequence were individually nucleofected into the WTC-LMNB1-mEGFP line (containing the CRISPRi-KRAB construct) using the Human Stem Cell Nucleofector Kit 1 solution with the Amaxa nucleofector 2b device (Lonza). Nucleofected cells were subsequently seeded at a density of 8,000 cells/cm² and recovered in mTESR1 media supplemented with ROCK inhibitor Y-276932 (10 uM) for two days. Guide selection was performed with blasticidin (10 ug/mL, ThermoFisher Scientific) for seven days, and clonal populations were generated through colony picking. Knockdown efficiency was evaluated through exposure to doxycycline (2 uM) for five days, after which mRNA was isolated, and relative levels of ZO1 were assessed through qPCR. Levels of ZO1 were normalized to copy numbers from the same line without CRISPRi induction.

The most effective was guide selected (ZO1_1 gRNA; CCGGTTCCCGGGAAGTTACG (SEQ ID NO:17)). After validation, this guide was subsequently introduced into the WTB CRISPRi-Gen1B line, which was selected and validated using the same methods.

### PGCLC Induction Using BMP-4 Colony Differentiation

To determine changes in proportions of germ lineage fates in Control (ZWT, ˜ DOX) and ZO1 KD (ZKD, +DOX) hiPSCs, unconfined colonies from each condition were treated with BMP-4 (50 ng/mL) in mTESR1 culture medium for 48 hours. The ZO1 knockdown cells were then stained for appropriate germ lineage markers. Note that for these experiments involving evaluation of the ability of monolayers and cell colonies to form PGCLCs, only ZO1 knockdown cells were used (because wild type cells in monolayers and colonies do not form PGCLCs without basolateral exposure to BMP).

Uniform colonies (˜100 ZO1 KD cells/colony) were achieved by seeding about 10,000 cells in mTESR1 supplemented with ROCK inhibitor Y-27632 (10 uM) from each condition into 400 by 400 mm PDMS microwell inserts (containing approximately 975 microwells) and force aggregating the cells through centrifugation at 200RCF for 3 minutes, using protocols adapted from those by Hookway et al., 2016; Ungrin et al., 2008 (FIG. 2B). After 18 hours, the aggregates were transferred in mTESR1 to Matrigel-coated 96 well plates at a density of approximately 10 aggregates/well. The cells were then allowed to attach and flatten into two dimensional (2D) colonies over the course of 24 hours prior to stimulation with BMP-4.

PGCLC Induction with BMP-4 Monolayer Differentiation

ZO1 wild type (ZWT) and ZO1 knockdown (ZKD) cells were seeded in mTESR1 supplemented with ROCK inhibitor Y-27632 (10 uM) into 96 well plates at a density between 100-350 cells/mm². The following day, the cells were fed with 100 ul-200 ul of mTESR1. On day 2, the cells were induced with BMP-4 (50 ng/ml) in mTESR1. At 48 and 72 hours after induction with BMP-4, the cells were fixed prior to staining for PGCLC and other somatic lineage markers. mRNA was collected from the 48 hour timepoint for qPCR analysis, the primers used for qPCR are listed in Tables 3-4.

TABLE 3
Primers for Pluripotency Genetic Markers

| Gene | First Primer | Second Primer |
|---|---|---|
| OCT4 | ATGCATTCAAACTG | AACTTCACCTTCCCTC |
| | AGGTGCCT (SEQ ID NO: 21) | CAACCA (SEQ ID NO: 22) |
| NANOG | CCCAAAGGCAAACAA | AGCTGGGTGGAAGAGA |
| | CCCACTTCT (SEQ ID NO: 23) | ACACAGTT (SEQ ID NO: 24) |
| DPPA3 | TGTTACTCGGCGGAG | GATCCCATCCATTAGA |
| | TTCGTAC (SEQ ID NO: 25) | CACGCAG (SEQ ID NO: 26) |
| SOX2 | AACCAGCGCATGGAC | CGAGCTGGTCATGGA |
| | AGTTA (SEQ ID NO: 27) | GTTGT (SEQ ID NO: 28) |
| PRDM14 | CCTTGTGTGGTATGG | CTTTCACATCTGTAGC |
| | AGACTGC (SEQ ID NO: 29) | CTTCTGC (SEQ ID NO: 30) |
| OTX2 | GGAAGCACTGTTTGCC | CTGTTGTTGGCGGCA |
| | AAGACC (SEQ ID NO: 31) | CTTAGCT (SEQ ID NO: 32) |
| SOX11 | GCTGAAGGACAGCGA | GGGTCCATTTTGGGC |
| | GAAGATC (SEQ ID NO: 33) | TTTTTCCG (SEQ ID NO: 34) |
| 18S | CTCTAGTGATCCCTG | ACTCGCTCCACCTCA |
| | AGAAGTTCC (SEQ ID NO: 35) | TCCTC (SEQ ID NO: 36) |

TABLE 4
Somatic/Germ Lineage Genetic Linkages

| Gene | First Primer | Second Primer |
|---|---|---|
| ZO1 | GCAGCTAGCCAGTGTA | GCCTCAGAAATCCAGC |
| | CAGTATAC (SEQ ID NO: 37) | TTCTCGAA (SEQ ID NO: 38) |
| T | TTTCCAGATGGTGAGA | CCGATGCCTCAACTCT |
| | GCCG (SEQ ID NO: 39) | CCAG (SEQ ID NO: 40) |
| NANOS3 | CCCGAAACTCGGCAG | AAGGCTCAGACTTCCC |
| | GCAAGA (SEQ ID NO: 41) | GGCAC (SEQ ID NO: 42) |
| BLIMP1 | CGGGGAGAATGTGGACT | CTGGAGTTACACTTGG |
| | GGGTAGAG (SEQ ID NO: 43) | GGGCAGC (SEQ ID NO: 44) |
| SOX17 | GAGCCAAGGGCGAGTCC | CCTTCCACGACTTGCCC |
| | CGTA (SEQ ID NO: 45) | AGCAT (SEQ ID NO: 46) |

PGCLC Induction with BMP-4 Transwell Differentiation

Corning Costar Transwell plates with a 6.5 mm diameter and 0.4 μm pore size (Cat. #07-200-147, Ref. #3414) were used. Transwell membranes were coated overnight with Matrigel. Prior to seeding, the Matrigel was removed and the membrane was rinsed 3× with PBS+/+ and then put into mTESR1 supplemented with ROCK inhibitor Y-27632 (10 uM). Cells were then immediately seeded onto the transwell membranes at a density of 500-1,500 cells/mm² (16,600-49,800 cells/well). Twenty-four hours later, ROCK inhibitor was removed, and the cells were fed with fresh mTESR1. Twenty-four hours after ROCK inhibitor removal, BMP-4 was added to both the apical (top) and basolateral (bottom) compartments. Forty-eight hours after BMP-4 induction, the transwells were fixed prior to staining for PGCLC and other somatic lineage markers (FIG. 3). Prior to imaging, the transwell membrane was removed and mounted onto a glass coverslip. 10 ng/mL BMP4 in transwells with a cell density of 750-1,000 cells/mm2 was optimal for PGCLC induction.

### Immunofluorescent Imaging

For staining, colonies and monolayers (plate or transwell) were fixed with 4% paraformaldehyde (VWR) for 20 minutes and subsequently rinsed 3× with PBS. Fixed cells were blocked and permeabilized for one hour at room temperature in 5% normal serum and 0.3% Triton™ X-100 (Sigma Aldrich) in PBS. Samples were then incubated with primary antibodies (still in staining buffer 5% normal serum/0.3% Triton™ X-100) overnight at 4° C. The following day, cells were rinsed 3× with PBS and incubated with secondary antibodies (1:400) in a 1% BSA, 0.3% Triton™ X-100 PBS solution. Primary and secondary antibodies used are listed in Table S.

TABLE 5
Antibodies for Immunofluorescent Staining

| Target | Species | Catalog Number | Supplier |
|---|---|---|---|
| BLIMP1 | Ms | MAB36081 | R&D |
| BMPR1A | Rb | 38-600 | ThermoFischer |
| CDX2 | Rb | 12306 | Cell Signaling |
| EOMES | Ms | MAB6166 | LEDQ0218092 |
| Ezrin | Ms | MA5-13862 | ThermoFischer |
| pSMAD1/5Oct4 | RbGt | 41D10, 9516sSC- | Cell Signaling |
| | | 8629 | Santa Cruz Biotech |
| SOX17pSMAD1/5 | GtRb | AF192441D10, | R&D Cell Signaling |
| | | 9516s | |
| SOX2SOX17 | RbGt | AB59776AF1924 | Abcam R&D |
| SOX2SOX2 | MsRb | 4900AB59776 | Cell Signaling Abcam |
| TBXTSOX2 | GtMs | AF20854900 | R&D Cell Signaling |
| ZO-1TBXT | MsGt | 33-9100AF2085 | Invitrogen R&D |
| ZO-1 | Ms | 33-9100 | Invitrogen |

### BMP4 Differentiation in Unconfined Colonies

To generate unconfined colonies of a defined size, PSCs were first force aggregated into 400×400 mm PDMS microwell inserts (24-well plate sized, ˜975 microwells/insert) using previously published protocols (Libby et al., bioRxiv 1-23 (2018); Hookway et al., Methods 101, 11-20 (2016); Ungrin et al., PLOS One 3, (2008)). Briefly, PSCs were dissociated, resuspended in mTESR1 supplemented with ROCK inhibitor (10 uM), seeded into the microwell inserts at a concentration of ˜50-100 cells/well, centrifuged at 200 relative centrifugal field (rcf) for 3 minutes, and left overnight to condense into aggregates. Next, the aggregates (˜50-100 cells in size) were resuspended in mTESR1 supplemented with ROCK inhibitor (10 uM) and transferred to Matrigel-coated 96 well plates at a concentration of approximately ˜15 aggregates/well, where they were allowed to attach and form 2D colonies. After 24 hours, ROCK inhibitor was removed and the colonies were fed with mTESR1. mTESR1 supplemented with BMP4 (200 ul/well, 50 ng/ml, R&D Systems) was added another 24 hours later to start the differentiation. Unconfined colonies of a defined size were also generated using an alternative protocol. Briefly, dissociated hPSCs were seeded at 2 cells/mm², and fed with mTESR1 supplemented with ROCK inhibitor for 4 days, after which they were fed for 2 days with regular mTESR1 or until they reached an appropriate size (approximately 300-500 um in diameter), after which they were treated with BMP4 as described above.

Transwell Culture of hPSCs and FITC Diffusion Assay

Corning Costar Transwell plates with a 6.5 mm diameter and 0.4 μm pore size (Cat. #07-200-147, Ref. #3414) were used. Transwell membranes were coated overnight with Matrigel. Prior to seeding, the Matrigel was removed and the membrane was rinsed 3× with PBS+/+ and then put into mTESR1 supplemented with ROCK inhibitor Y-27632 (10 uM). Cells were then immediately seeded onto the transwell membranes at a density of 1,500 cells/mm² (49,800 cells/well). 24 hours later the ROCK inhibitor was removed, and the cells were fed with fresh mTESR1. 24 hours after ROCK inhibitor removal, the membranes were imaged on an EVOS fluorescence microscope at 10× to visualize whether the GFP labelled cellular nuclei reached confluence and were completely covering the membrane. The inventors had previously determined that this protocol generates intact epithelia at this timepoint.

To visualize pSMAD1 activity in BMP4 stimulated transwells over time, BMP4 (50 ng/ml) was added to either the apical (top) or basolateral (bottom) compartments of the transwell. The transwells were fixed at the appropriate time points by transferring the insert to a new 24 well plate, rinsing with PBS, and fixing with 4% PFA.

To perform the FITC diffusion assay, FITC conjugated to 40-kDa dextran (Sigma-Aldrich) was added to the apical compartment and 10 ul of media was collected from basolateral compartment at various timepoints, which was mixed with 90 ul of PBS onto a 96-well dark-sided plate. After the time course was completed, a plate reader was used to take fluorescence measurements of our samples over time.

### Immunofluorescent Staining and Marker Quantification

Human PSCs were rinsed with PBS 1×, fixed in 4% paraformaldehyde (VWR) for 15 minutes, and subsequently washed 3× with PBS. The fixed cells were permeabilized and blocked in 0.3% Triton X-100 (Sigma Aldrich) and 5% normal donkey serum for an hour, and then incubated with primary antibodies overnight (also in 0.3% Triton, 5% normal donkey serum). The following day, samples were washed 3× with PBS and incubated with secondary antibodies in 0.3% Triton and 1% BSA at room temperature for 2 hours. Secondary antibodies used conjugated with Alexa 647, Alexa 405, and Alexa 555 (Life Technologies), and were used at a dilution of 1:400.

### RNA Sequencing

ZO1 wild type (ZWT) and ZO1 knockdown (ZKD) cells were seeded at a density of 250 cells/mm² onto standard culture 6-well plates in mTESR1 supplemented with ROCK inhibitor (10 uM). 24 hours later, ROCK inhibitor was removed, and the cells were fed with fresh mTESR1. 24 hours after ROCK inhibitor removal, cell lysates for the pluripotent condition were prepared by putting 1.5 mL RLT (lysis) buffer/well for 3 minutes, and freezing this lysate at −80° C. for subsequent RNA extraction. Simultaneously, BMP4 (50 ng/ml) was added to the differentiated condition. After 48 hours of BMP4 treatment, cell lysates for the differentiated condition were prepared as described above. RNA extraction was performed using Qiagen's RNBasy kit, and samples were subsequently shipped to Novogene for library preparation and sequencing (Illumina, PE150, 20M paired reads).

### Whole Genome Bisulfite Sequencing

ZO1 wild type (ZWT) and ZO1 knockdown (ZKD) cells were seeded and cultured as described in the RNA sequencing section. Only pluripotent samples were sent for sequencing. To do this, cells were dissociated using Accutase and resuspended in 200 ul PBS+proteinase K, and then frozen at −20° C. for subsequent DNA extraction. DNA extraction was performed using Qiagen's DNA extraction kit. Samples were subsequently sent to CD Genomics for whole genome bisulfite sequencing (Illumina, PE150, 250M paired reads).

### Example 2: ZO1-Knockdown and BMP to Make PGC Like-Cells (PGCLCs)

This Example illustrates generation of primordial germ-like cells (PGCLCs) from hiPSC cells modified to knockdown ZO1.

A doxycycline (DOX)-inducible CRISPR interference system was made for integration into the WTB (female) and WTC (male) parent hiPSC lines (FIG. 1A). The CRISPR interference system was comprised of two components: a dCas9KRAB repressor driven by a TetO promoter that was inserted into the AAVS1 safe harbor locus and that is expressed only under DOX treatment, and a constitutively expressed guide RNA (gRNA) that targets the transcriptional start site of the ZO1 gene. The ZO1-specific gRNA (Table 2; FIG. 1A) was encoded in a randomly-integrating plasmid that also expressed a blasticidin selection gene. DOX-inducible expression of Cas9 enabled temporal control of its gene expression. These constructs were transfected into both the WTB and WTC hiPSC CRISPRi cell lines. Knockdown of ZO1 was achieved after 5 days of DOX treatment in cells cultured in mTESR on Matrigel coated plates (seeding density 120 cells/mm²). The cells were passaged every three days using Accutase for cell displacement. hPGCLC induction was commenced by adding 50 ng/mL BMP4 directly to a monolayer of ZO1 knockdown hiPSCs at seeding densities between (100-2000 cells/mm²) for at least two days.

As illustrated in FIG. 1B-1C, reduced expression of ZO1 was observed in the cells within one day of DOX treatment, and ZO1 expression became minimal by day 5 after DOX was introduced into the culture medium.

To evaluate the barrier function and ability of ZO1 knockdown cells to preclude diffusion of molecules from one side of a cellular monolayer to the other, an assay was performed that involved growing the wild type or ZO1 cells on a transwell membrane where both apical and basolateral sides are independently accessible. The apical side was treated with 40 kDa FITC (dextran molecules conjugated with the fluorescent molecule FITC), and media from the basolateral side was sampled over time for fluorescent measurements to determine permeability of the cell layer. FIG. 1D show that ZO1 knockdown results in loss of tight junction barrier function as measured by FITC-Dextran diffusion. Hence, apical to basolateral diffusion is disrupted by ZO1 knockdown.

Wild type and ZO1-knockdown cells that were maintained in transwells were treated for 5 days with Doxycycline (2 uM) and the transepithelial electrical resistance (TEER) of the cells was measured. As shown in FIG. 1E, ZO1 knockdown cells exhibit loss of transepithelial resistance, indicating ZO1 knockdown results in loss of barrier function.

FIG. 2A illustrates that when BMP4 is provided basolaterally (diagram inset), pSMAD1 expression is activated whether or not ZO1 expression is knocked down (see top row of images). However, when BMP4 is provided apically, pSMAD1 expression is not activated when ZO1 is expressed (FIG. 2A, bottom left panels). However, pSMAD1 expression is activated when ZO1 is not expressed (FIG. 2A, bottom right images).

FIG. 2B illustrates methods tested for generating PGCLCs from pluripotent stem cells. Knock down (KD) of ZO1 expression is not necessary for generating PGCLCs when BMP4 is provided basolaterally in a culture medium such as mTESR (FIG. 2B top row with BMP4 on the bottom row). However, ZO1 knockdown (KD) can be used to facilitate PGCLC generation by DOX-induced KD (FIG. 2B, middle row). Addition of BMP4, especially basolateral addition of BMP4, to ZO1 knockdown PSCs can also generate PGCLCs.

Moreover, the cells need not be aggregated and can just be seeded directly onto Matrigel coated plates and stimulated with BMP4 for 48 hours. FIG. 2C˜2E show successful differentiation of ZO1 KD hiPSCs to PGCLCs, using both aggregation and monolayer differentiation methods.

### Example 3: Generating Primordial Germ Cells without Genetic Modification

This Example describes methods for differentiating pluripotent stem cells (PSCs) to primordial germ cells like cells (PGCLCs), where the pluripotent stem cells (PSCs) are not genetically modified, or chemically treated (except for the addition of ROCK inhibitor to promote survival after seeding).

One day prior to dissociating the PSCs, Matrigel was coated onto the transwell membranes, and left at 37° C. overnight. The next day, pluripotent stem cells (PSCs) growing in mTESR medium were dissociated with Accutase and resuspended in mTESR with 10 uM ROCK inhibitor. Matrigel was aspirated off of the transwell membranes and the apical and basolateral compartments were filled with mTESR+10 uM ROCK inhibitor. The PSCs were seeded at a density of 1000 cells/mm² onto the transwell membrane, however in some cases, the number of seeded PSCs can be varied. The following day, the spent media was aspirated, and mTESR media was added. The day after that, mTESR media was added to the apical compartment, and mTESR media with 5-50 ng/mL BMP4 was added to the basolateral compartment, as shown in FIG. 3 in the rightmost panel. 10 ng/mL BMP4 was found to be optimal for PGCLC induction. PGCLCs could be harvested starting at Day 2, but the cells can be incubated with daily changes of differentiation media up until Day 6 to increase cell yield.

### Example 4: BMP Pathway Activation Correlates with Regional Loss of ZO1

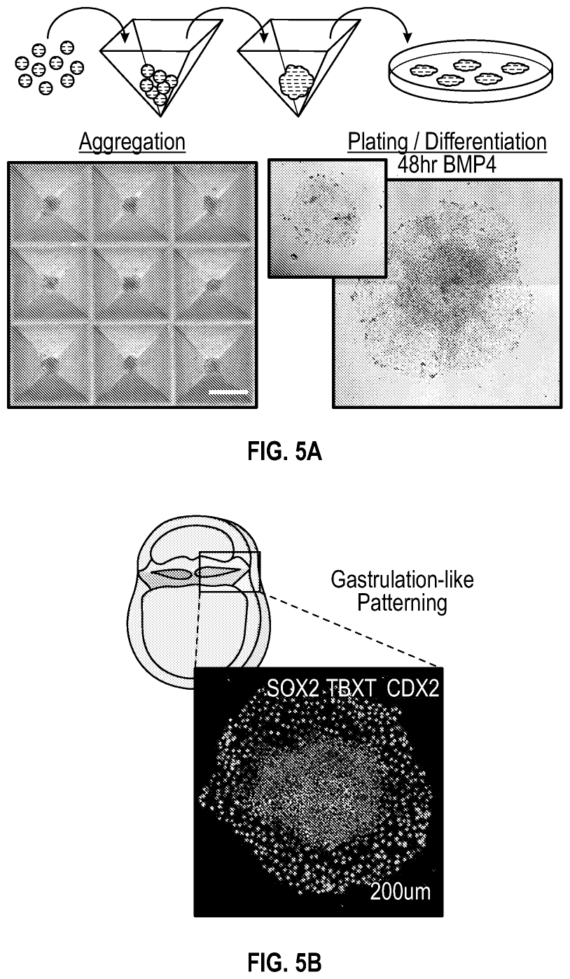

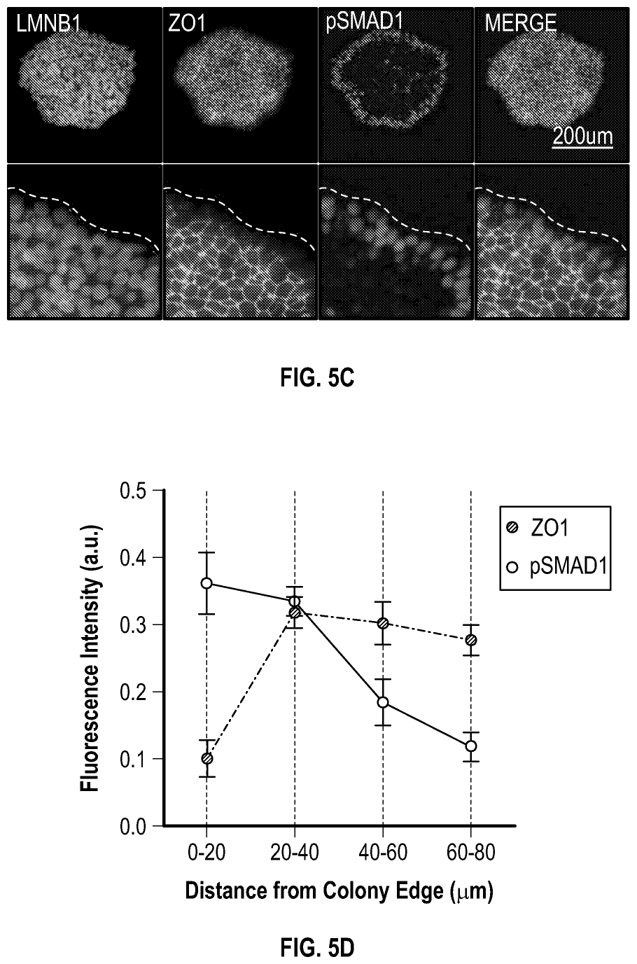

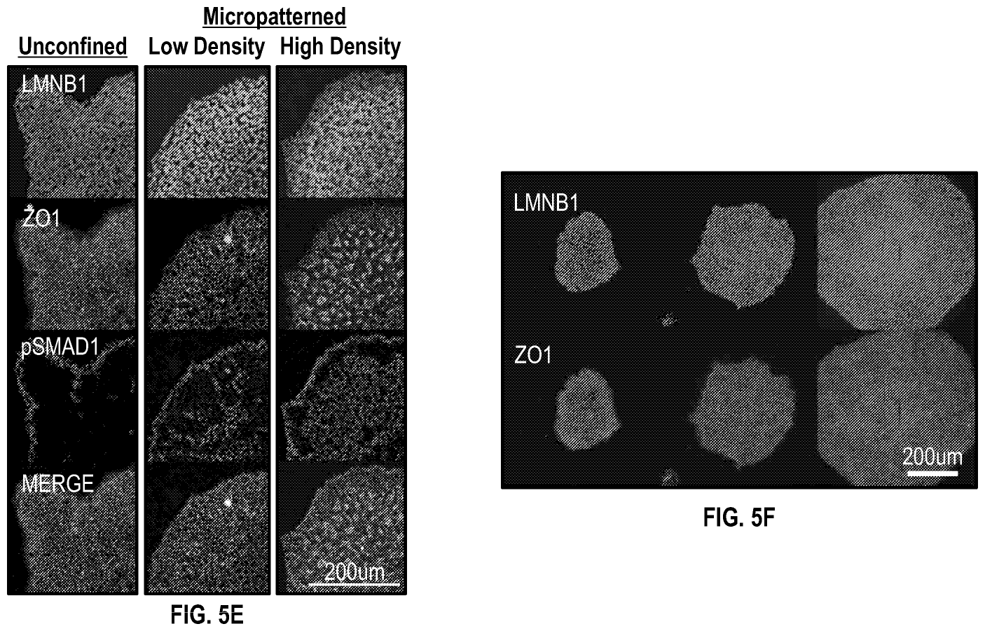

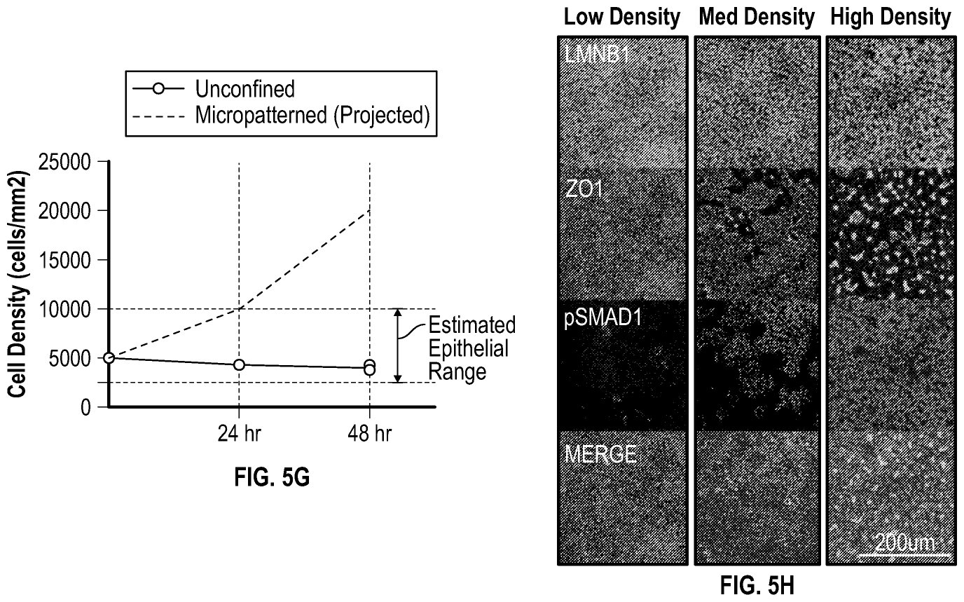

Human PSCs confined to circular micropatterns and treated for 42-48 hours with BMP4 undergo radial patterning of gastrulation-associated makers CDX2 (trophectoderm-like), TBXT (mesendoderm-like), and SOX2 (ectoderm-like), specified radially inward from the colony border. The inventors and others have demonstrated that similarly-sized colonies whose growth is not confined by micropatterns undergo analogous radial patterning in response to BMP4 stimulation (Libby et al., bioRxiv 1-23 (2018); Joy et al. Stem Cell Reports 16, 1317-1330 (2021); Gunne-Braden et al., Cell Stem Cell 26, 693-706.e9 (2020)) (FIG. 5A-5B). In this modified protocol, human pluripotent stem cells were aggregated overnight within pyramidal microwells, and the following day these 3D aggregates are re-plated sparsely and allowed to grow into distinct 2D colonies 300-500 um in diameter. This system was utilized because, compared with micropatterned colonies, unconfined colonies maintain a relatively uniform density and a robust epithelial morphology over time (FIG. 5E-5G). This is important because epithelial integrity is a direct function of cell density; previous reports have linked changes in signaling and cell specification with changes in cell density (Etoc et al., Dev. Cell 39, 302-315 (2016); Nallet-Staub et al., Dev. Cell 32, 640-651 (2015); Smith et al., Proc. Natl. Acad. Sci. U.S.A. 115, 8167-8172 (2018); Manfrin et al., Nat. Methods 16, 640-648 (2019)).

Low cell densities can prevent proper tight junction formation and presumably enhance permeability to signaling proteins (Etoc et al., Dev. Cell 39, 302-315 (2016). Interestingly, the inventors have discovered that the opposite is also true: in monolayer culture at high cell densities, the honeycomb-like intercellular protein expression pattern of ZO1, which is indicative of an intact epithelium, becomes disrupted and punctate (FIG. 5H). Regions with punctate ZO1 expression, which increase in frequency as cell density increases, overlap with regions of BMP4-induced signaling pathway activation (phosphorylation of SMAD1). This suggests that very low and very high cell densities can both cause increases in epithelial permeability. In our hands, this phenotype is also present in micropatterned colonies, regions of high density lose ZO1 and overlap with pSMAD1 activation upon BMP4 stimulation (FIG. 5F). Discrepancies in previously reported pSMAD1 pre-patterns may therefore be explained in part to regional changes in density and consequent effects on epithelial structure.

Interestingly, ZO1 expression inversely correlates with pSMAD1 activation even in the context of unconfined colonies with uniform density. For example, at early timepoints upon induction with BMP4, pSMAD1 activity is largely limited to the edge of colonies. ZO1 expression does not fully extend to the edge of the colony, and tapers off a distance of approximately one cell layer before reaching the edge.

Co-staining of ZO1 and pSMAD1 in unconfined colonies after 1 hour of BMP4 stimulation exhibited an anti-correlation between pSMAD1 positive and ZO1 positive regions (FIG. 5C)—cells expressing pSMAD1 did not also express ZO1. Quantification of fluorescent signal normalized to nuclear LMNB1 expression at different distances from the colony edge further demonstrated the inverse relationship between pSMAD1 and ZO1 (FIG. 5D). Initial pSMAD1 pre-patterning has been implicated in regulating subsequent gastrulation-associated patterning in micropatterned colonies. The inventors have conducted the experiments described herein to elucidate the effect of tight junctions on signaling and gastrulation patterning.

### Example 5: ZO1 Knockdown Leads to Ubiquitous and Sustained Pathway Activation

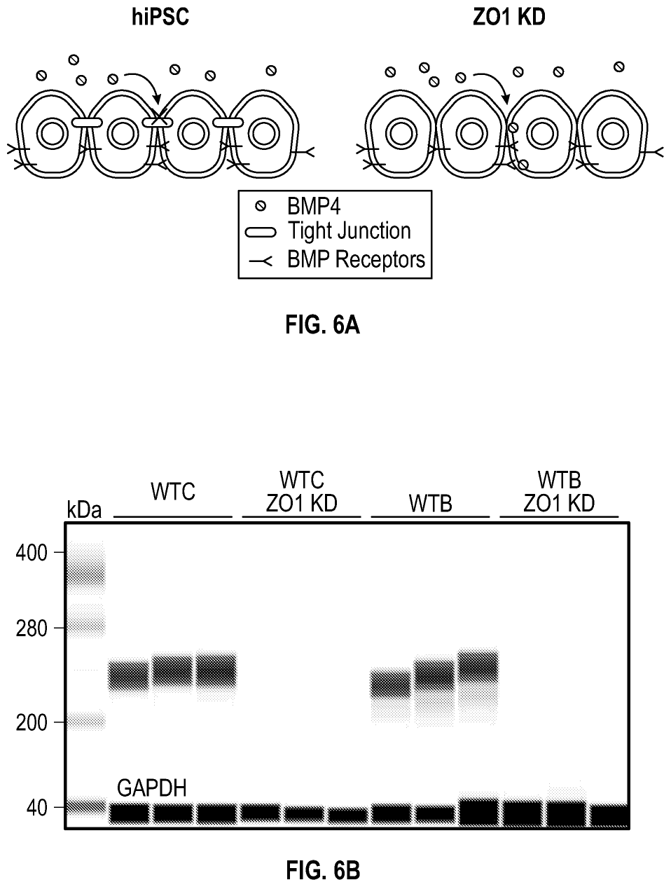

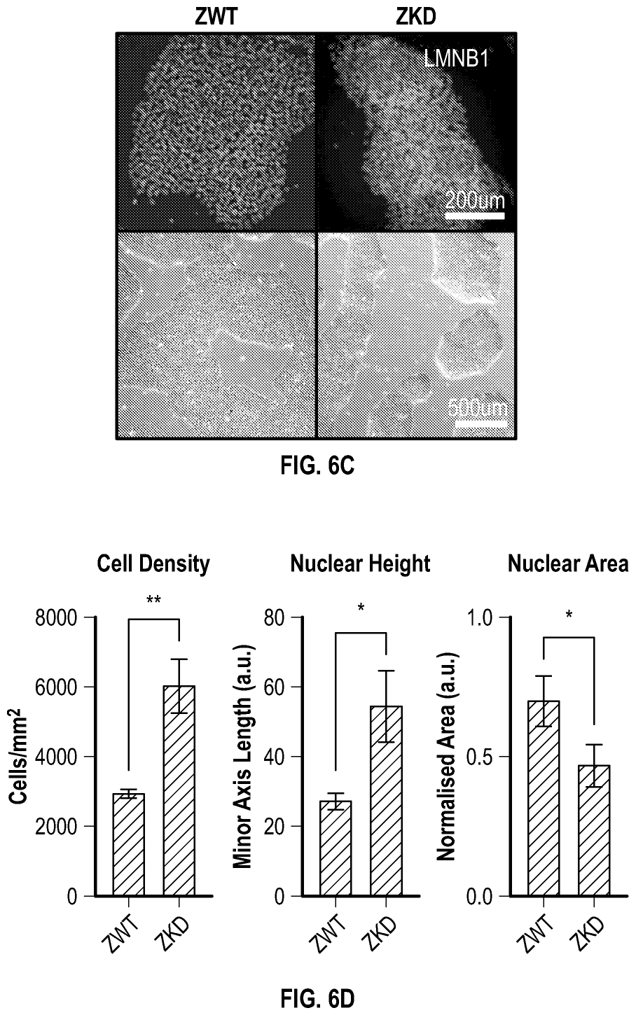

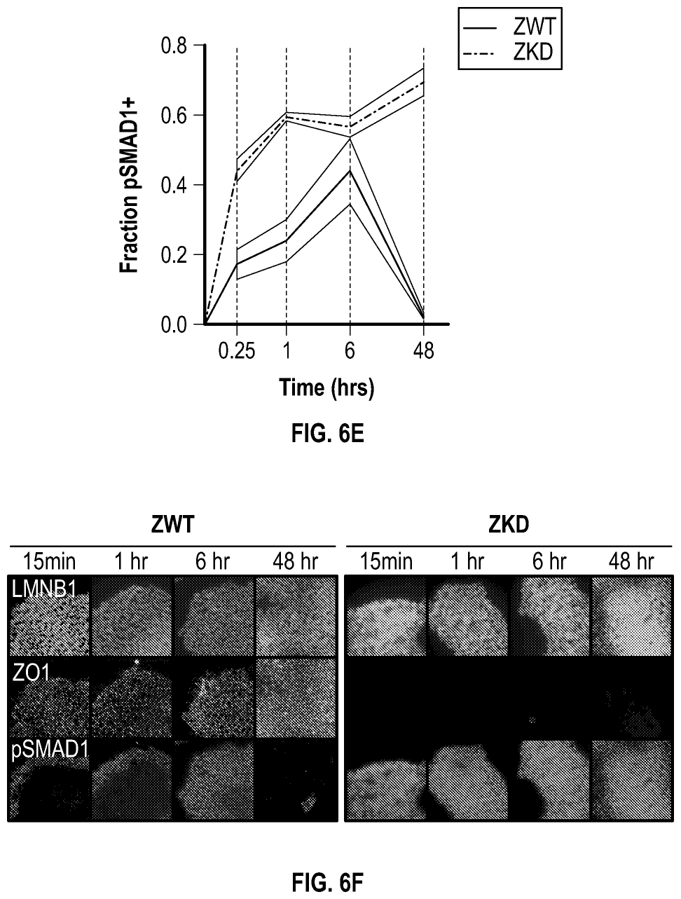

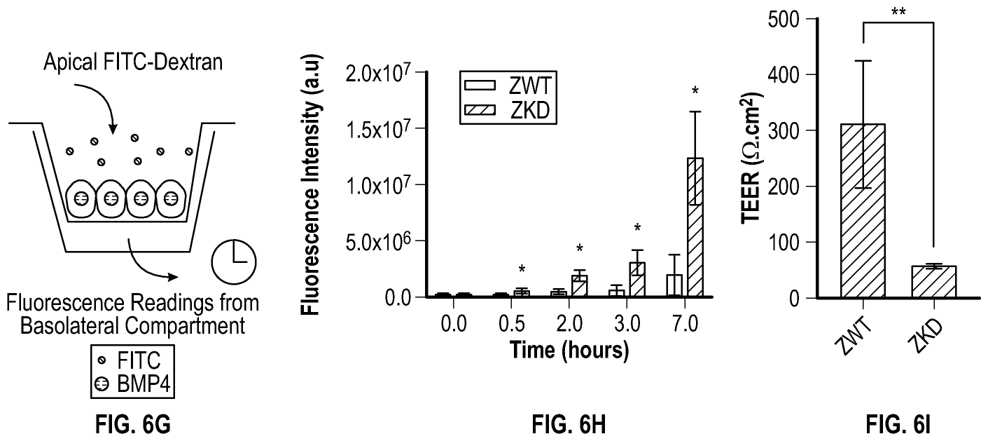

In vitro hPSCs cultured as epithelial sheets that have tight junctions and display apical/basolateral polarity, with most morphogen receptors, including BMP receptors BMPR1A, BMPR2, and ACVR2A, localized to the basolateral side. These receptors are physically partitioned away from morphogens presented in the media on the apical side. As a result, tight junction expression presumably attenuates cellular response to exogenous morphogen signals in vitro (FIG. 6A).

In order to explore how tight junctions affect signaling in the unconfined colonies, the DOX inducible CRISPR interference (CRISPRi) system was used to knockdown ZO1 (FIG. 1A). ZO1 was specifically targeted because preliminary RNA sequencing data showed that ZO1 is much more highly expressed in cultured hPSCs than ZO2 or ZO3 (data not shown). Both male (WTC) and female (WTB) hPSC ZO1 knockdown lines were created. The WTC line also contained a LMNB1-GFP fusion reporter for live nuclear visualization. Both hPSC ZO1 CRISPRi lines were karyotypically normal (FIGS. 1G-1H), and RNA and protein expression are significantly depleted after five days of DOX treatment, as shown by qPCR, immunofluorescence (IF), and western blot (FIGS. 1B-1C, 6B). Most of the characterization in the WTC ZO1 CRISPRi line was performed with and without DOX (referred to in the text as ZO1 wild type (ZWT) and ZO1 knockdown (ZKD), respectively), however, the results for the WTB ZO1 CRISPRi line were phenotypically similar and reproducible.

ZO1 knockdown cells grew in somewhat denser colonies and exhibited rounder nuclear shapes (FIG. 6C-6D). Where ZO1 wild type nuclei are stretched and flat, ZO1 knockdown nuclei are taller and more rounded, likely as a result of severed connections between the cell-cell junctions and the actin cytoskeleton/nuclear lamina.

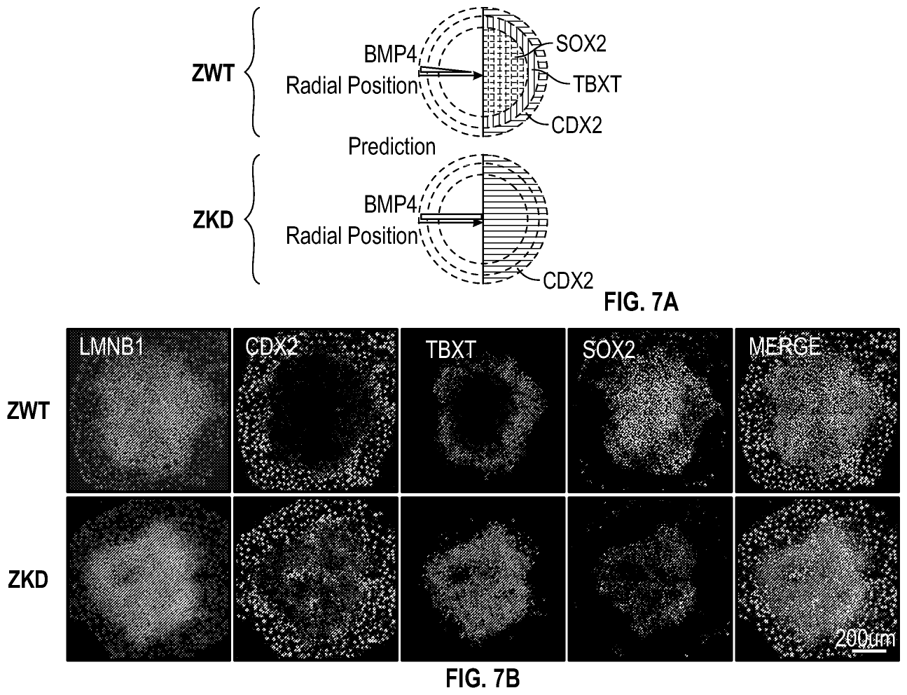

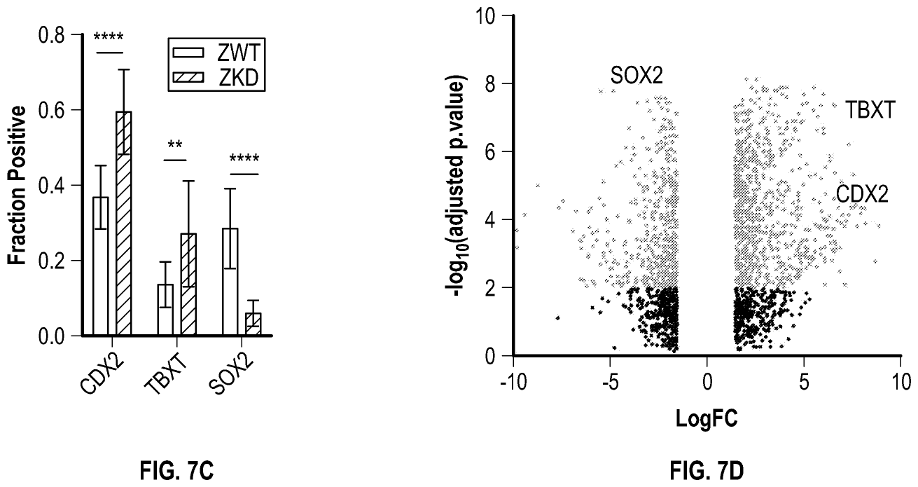

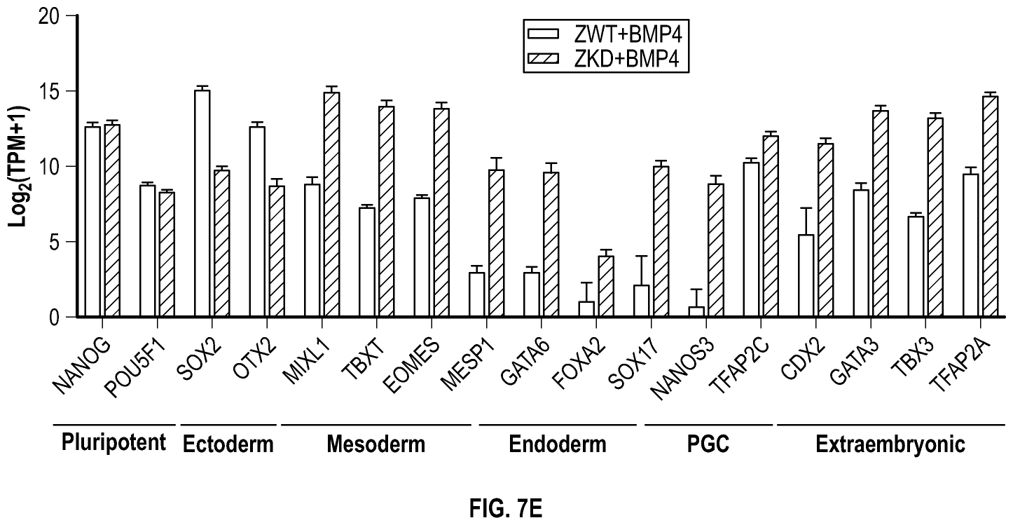

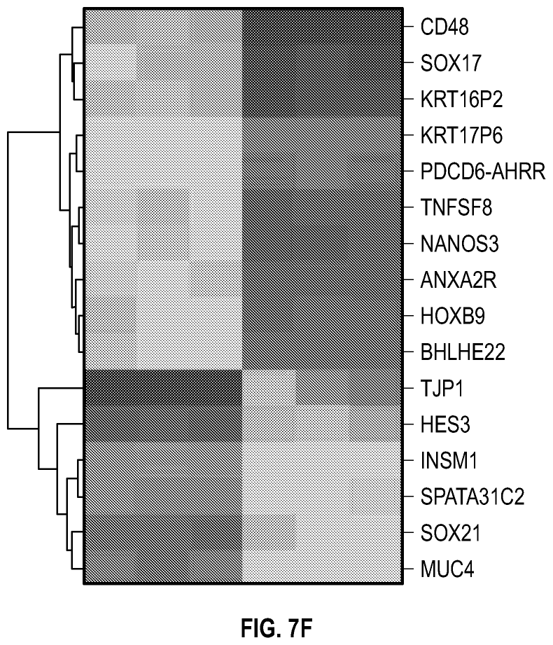

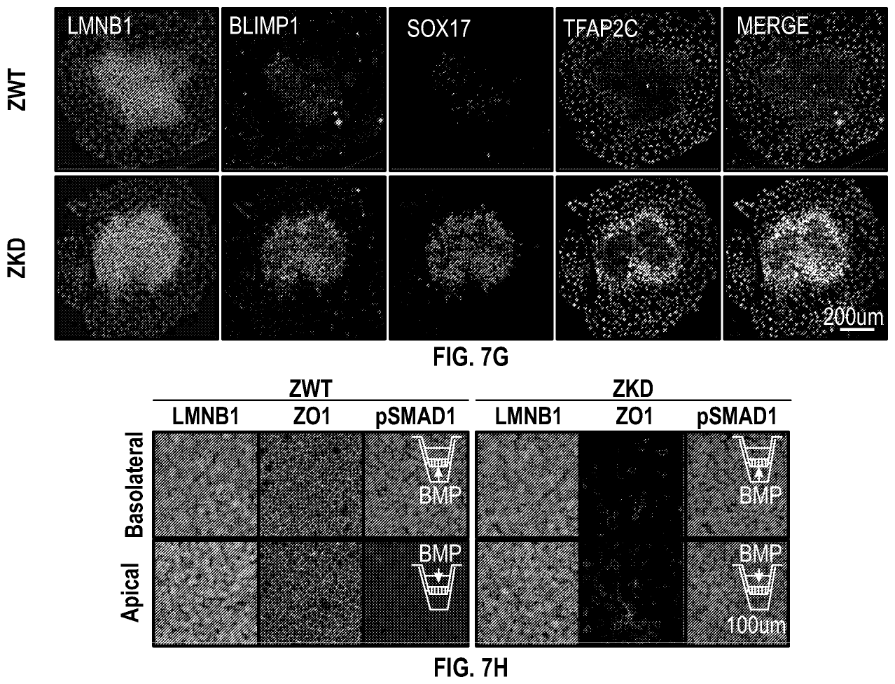

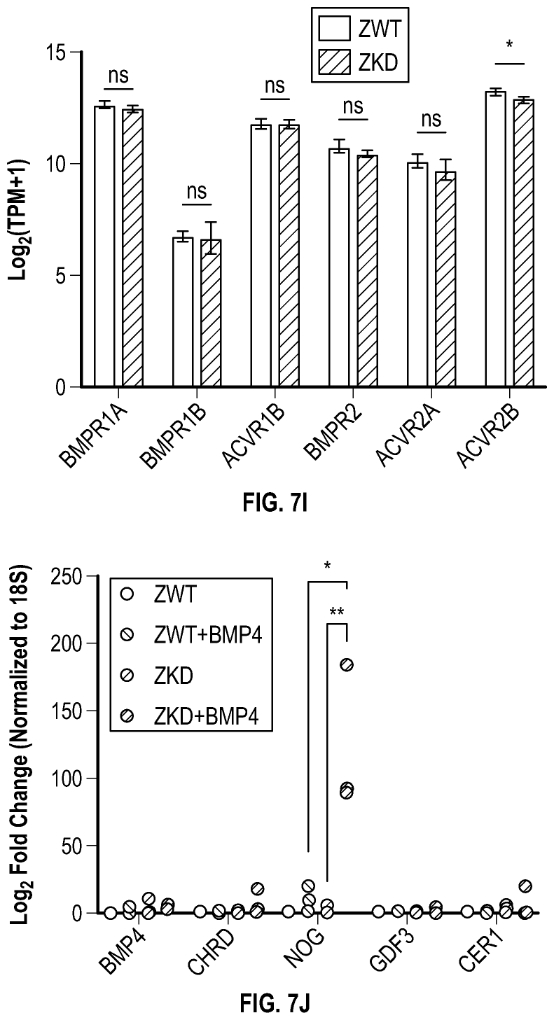

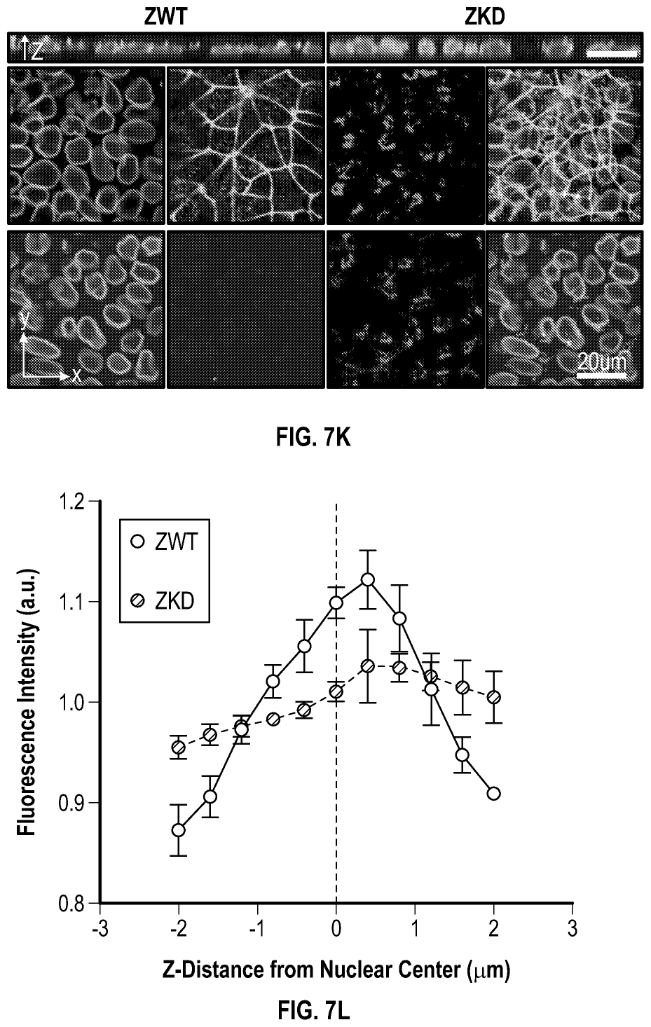

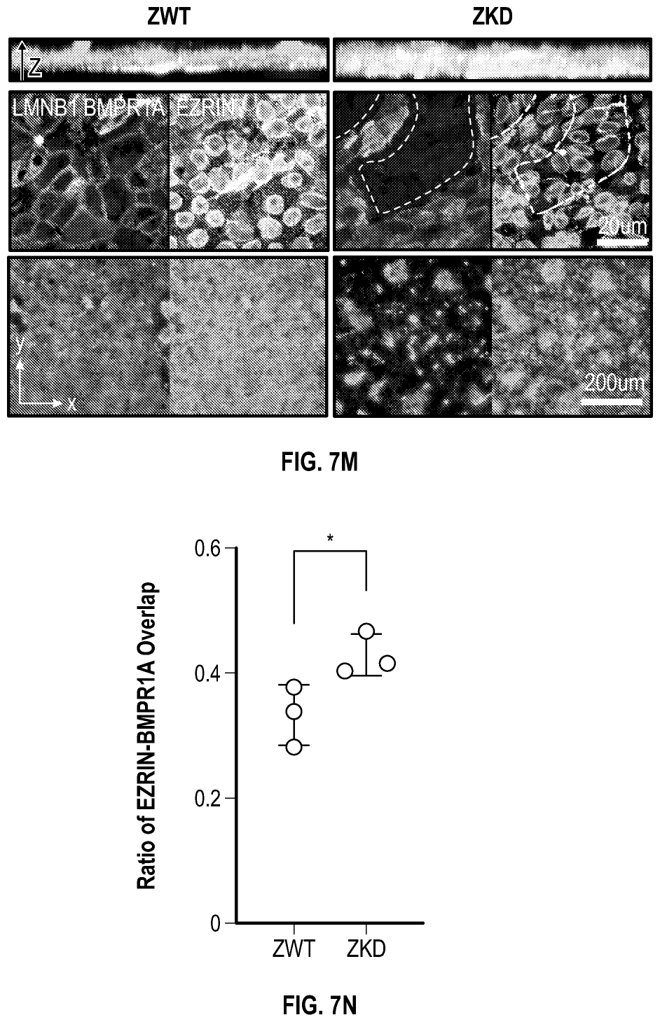

When grown as unconfined colonies and exposed to BMP4, ZO1 wild type largely limited pSMAD1 expression to the colony edge at early timepoints (15 min-1 hr) (FIG. 5C-SD). At later timepoints (6 hours), pSMAD1 is detectable in cells located centrally within the colony. However, pSMAD1 expression is subject to inhibitor feedback loops. Thus, this pathway activation is shut off by 48 hours in ZO1 wild type cells (FIG. 6E-6F). Strikingly, at early timepoints, the ZO1 knockdown colonies displayed pSMAD1 throughout the colony (FIG. 6F). Furthermore, ZO1 knockdown cells maintain pSMAD1 activation over time (FIG. 6F), despite significant increases in transcription of the secreted BMP inhibitor NOGGIN (FIG. 7J), which is implicated in driving SMAD1 pathway inactivation in ZO1 wild type cells over time. In ZO1 wild type cells, NOGGIN is secreted apically and is trafficked transepithelially with assistance from glycoproteins on the apical surface.

The observed maintenance of pSMAD1 pathway activation despite increase in NOGGIN in ZO1 knockdown colonies indicates that ZO1 is not only important for preventing ligands such as BMP4 from accessing basolateral receptors, but may also be necessary in rendering the cells sensitive to some inhibitors, presumably by maintaining expression of the apical surface glycoproteins that enable transepithelial trafficking of apically secreted inhibitors such as NOGGIN or sequestration/concentration of other basolaterally secreted morphogen inhibitors within the colony interior. This observation is reinforced by the fact that ZO1 knockdown cells also exhibit loss of apical Ezrin expression (FIG. 1F), which can be important in tethering apical glycoproteins to the actin cytoskeleton.

### Example 6: Signaling Changes Result from Increased Permeability in ZO1 Knockdown Cells

In order to confirm basolateral sequestration of BMP receptors within an epithelium, cells were grown on a transwell membrane, where both apical and basolateral sides of the media are accessible. Using transwells allows for unidirectional exposure of BMP4 from either cellular domain. As early experiments have indicated, basolateral presentation of BMP4 is required for pSMAD1 activation in ZO1 wild type cultures. Alternatively, both apical and basolateral stimulation activates pSMAD1 in ZO1 knockdown (ZKD) cells (FIG. 7H). ZO1 wild type and ZO1 knockdown cells do not have differences in BMP4 receptor expression (FIG. 7I). Several possibilities could explain this phenomenon: ZO1 knockdown causes mixing of apical/basolateral domain elements through the plasma membrane and disrupted trafficking of receptors to their proper domains (loss of apical/basolateral polarity), or ZO1 knockdown causes increased permeability to signaling molecules (loss of barrier function). To test these possibilities, the inventors first characterized apical/basolateral polarity between ZO1 wild type and ZO1 knockdown cells.

In polarized cells, the Golgi apparatus faces the apical (secretory domain) direction. Therefore, the inventors evaluated positioning of the Golgi in ZO1 wild type and ZO1 knockdown cells. Z-stacks revealed that in both cell types, the Golgi sits on top of the nucleus on the apical side of the cell, suggesting that polarity of the ZO1 knockdown cells is still intact (FIG. 7K-7L). However, staining for the apical marker Ezrin revealed significant eradication of the apical domain in ZO1 knockdown cells, characterized by punctate Ezrin localization. This is consistent with previous reports that Ezrin is lost on the colony edge of regular hPSC colonies (Kim et al., Stem Cell Reports 17, 68-81 (2022)). Immunofluorescence images showed that swaths of ZO1 knockdown cells lost apical Ezrin; and even in regions where Ezrin is present, it overlaps significantly with BMPR1A (a basolateral BMP receptor), indicating potential changes in localization of some apical/basolateral elements (FIG. 7M-7N). Our results indicate that polarity-associated changes do not occur in cytoplasmic elements, but may occur for elements bound to the plasma membrane.

FITC based diffusion assay was performed to look for differences in permeability in ZO1 wild type and ZO1 knockdown. Each cell type was grown on a transwell membrane and a 40 kDa dextran conjugated with FITC was added to the apical compartment (FIG. 6G). The 40 kDa-FITC was selected due to its similarity in hydraulic radius to many common gastrulation-associated signaling proteins. Specifically, 40 kDa-FITC is slightly smaller than BMP4. Hence, an epithelial barrier that could exclude the 40 kDa-FITC is evidence that the epithelial barrier could also exclude BMP4.

Fluorescence measurements of the basolateral compartment over time were used to quantify permeability of the ZO1 knockdown cells compared to the control. As shown in FIG. 6F, significant increases in passage of FITC through ZO1 knockdown cell layers could be observed as early as 30 minutes into 40 kDa-FITC treatment. Similarly, trans epithelial resistance (TEER) measurements performed on control and ZO1 knockdown monolayers confirmed that ZO1 knockdown cells are not able to form a true epithelium that resists passage of ions through the paracellular space (FIG. 6I). Therefore, while some changes in apical/basolateral polarity may occur, the results described herein indicate that definitive changes in permeability drive heightened signaling pathway activation seen in ZO1 knockdown cells.

### Example 6: ZO1 Knockdown Causes Changes in Cell Fate Proportions in Unconfined Gastrulation Models

Several models have been proposed to explain how multiple distinct lineages can arise in a colony exposed to a uniform dose of BMP4. The current paradigm combines the principles of Alan Turing's reaction diffusion (RD) (Turing, Philos. Trans. R. Soc. 37-72 (1952)) and Lewis Wolpert's positional information (PI) (Wolpert, J. Theor. Biol. 25, 1-47 (1969); Green & Sharpe, Dev. 142, 1203-1211 (2015)). The RD model proposes that in response to signal pathway activation (phosphorylation of SMAD1) by an activating species (BMP4), cells secrete more of this activator (BMP4) and its inhibitor (NOGGIN) in a feedback loop (Tewary et al., Development dev. 149658 (2017)). Differences in the diffusivities between NOGGIN and BMP4 can create a steady-state gradient of effective BMP4 concentrations across the colony, and cells sense positional information and differentiate based on both on this concentration gradient and its overlap with other members of a BMP4-induced feedback loop, including WNT and NODAL. The initial pSMAD1 pre-pattern is therefore assumed to be an important indication of the shape of an RD gradient which determines the shape of subsequent gastrulation-associated patterning.

In ZO1 wild type, this temporal pSMAD1 profile is reserved for cells on the edge of colonies that remain pSMAD1 positive throughout BMP4 stimulation and eventually acquire CDX2+ trophectoderm-like fates. By contrast, ZO1 knockdown cells maintain ubiquitous and sustained pSMAD1 activation throughout the entire colony. Therefore, if the current RD/PI paradigm is correct, the inventors predicted that ZO1 knockdown cells would ubiquitously differentiate to the CDX2 lineage (FIG. 7A). Accordingly, these results show that ZO1 knockdown colonies treated with BMP4 have increased CDX2 expression across the colony interior. In addition, these ZO1 knockdown colonies display a stark decrease in central SOX2 expression, and disruption of the TBXT ring pattern (FIG. 7B-7C). These results establish ZO1, and therefore tight junction stability, as a key component of BMP4-induced cell fate and spatial patterning.

### Example 7: RNA Sequencing of BMP4-Treated ZO1 Knockdown Colonies Reveals PGCLC Bias

Unexpectedly, the inventors also observed that like CDX2, TBXT expression is substantially increased throughout the center of the colony (FIG. 7B). Many progenitor cell types express TBXT. To better identify this TBXT-expressing population and quantify changes in ZO1 knockdown induced lineage bias, RNA sequencing was performed on pluripotent and 48-hour BMP4 treated ZO1 wild type and ZO1 knockdown cells.

RNA sequencing confirmed the immunofluorescence staining results: CDX2 and TBXT transcripts are upregulated, whereas SOX2 is downregulated (FIG. 7D). Analysis of a panel of well-known gastrulation associated lineage markers in ZO1 wild type and ZO1 knockdown cells revealed that ZO1 knockdown cells have the tendency to express mesendoderm, PGC, and extraembryonic markers at the expense of ectodermal-like lineages (FIG. 7E).

Gene ontology (GO) analysis performed on Clusters 2 and 3 of the top 150 differentially expressed genes between ZO1 wild type and ZO1 knockdown cells shows upregulation of endoderm and sex cell related pathways in ZO1 knockdown colonies, as illustrated in Table 6 below.

TABLE 6
Gene Sets Enriched in ZO1 Knockdown Cells

| Gene-set Enriched GO Terms | FDR |
|---|---|
| Cluster 2: | |
| Endodermal cell differentiation | 4.62E−02 |
| Mesoderm formation | 1.49E−04 |
| Embryonic placenta development | 2.23E−02 |
| Cell migration involved in gastrulation | 1.75E−04 |
| Trophectodermal cell differentiation | 1.41E−02 |
| Cluster 3: | |
| Endodermal cell fate determination | 7.99E−03 |
| Embryonic foregut morphogenesis | 1.60E−03 |
| Reproductive system development | 5.79E−03 |
| Sex differentiation | 1.95E−03 |
| Germ cell migration | 3.07E−02 |

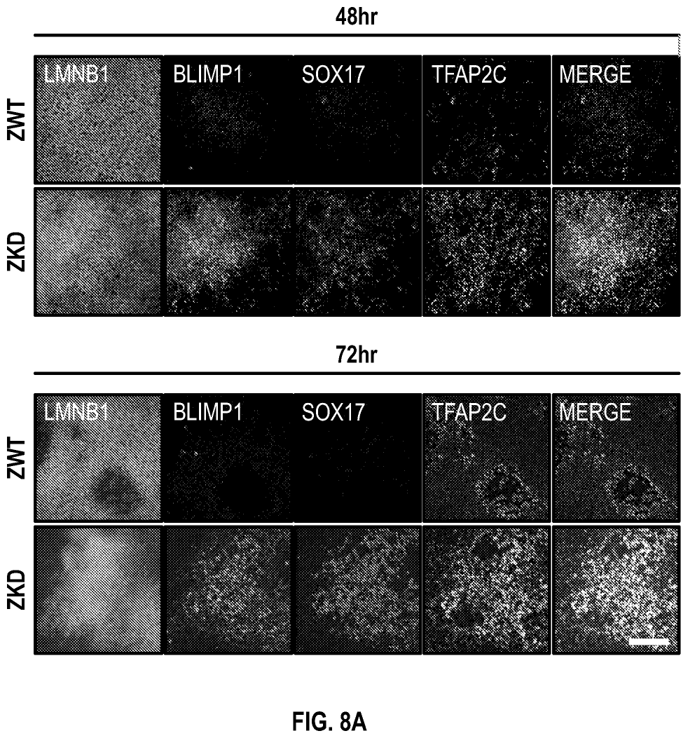

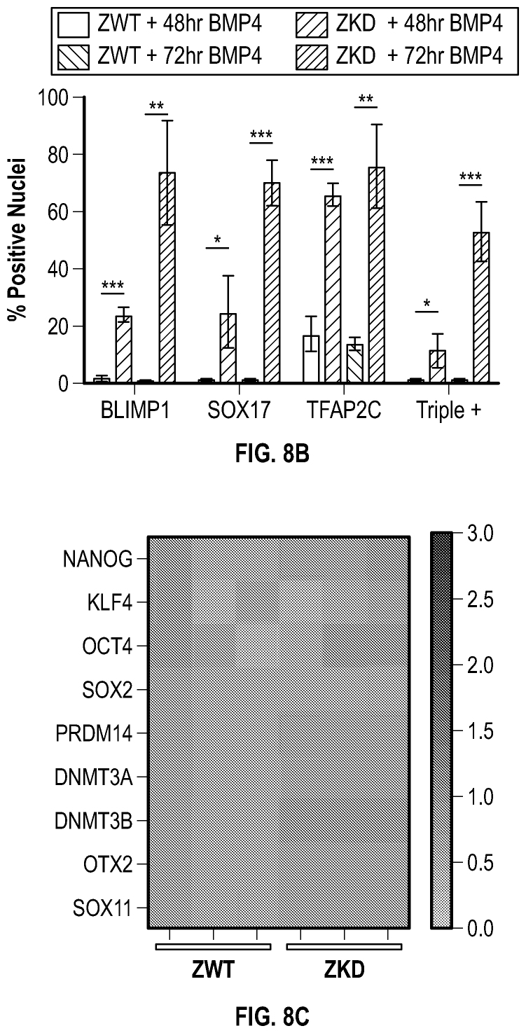

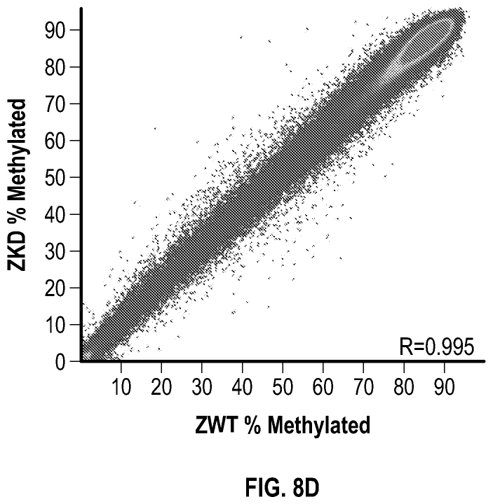

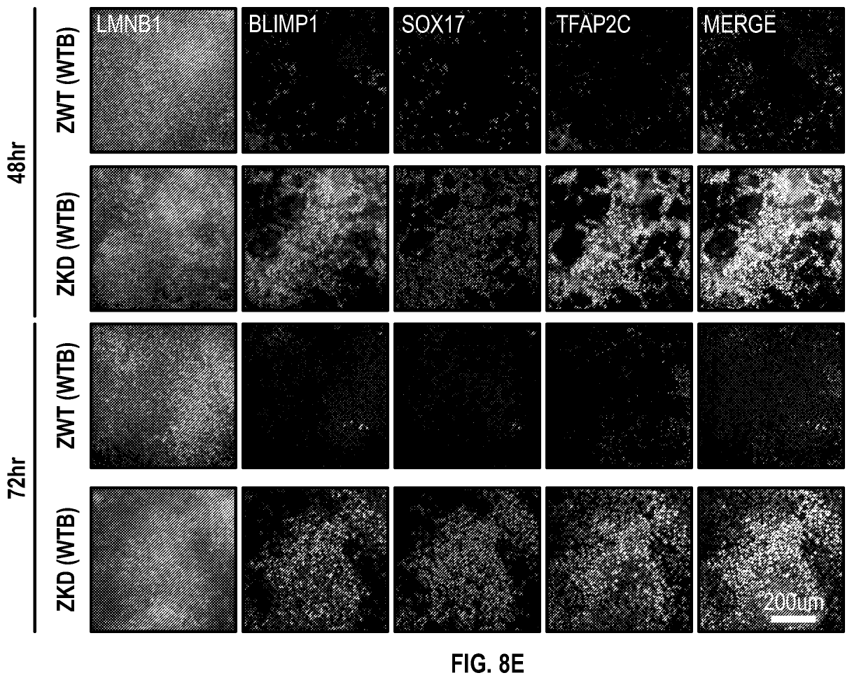

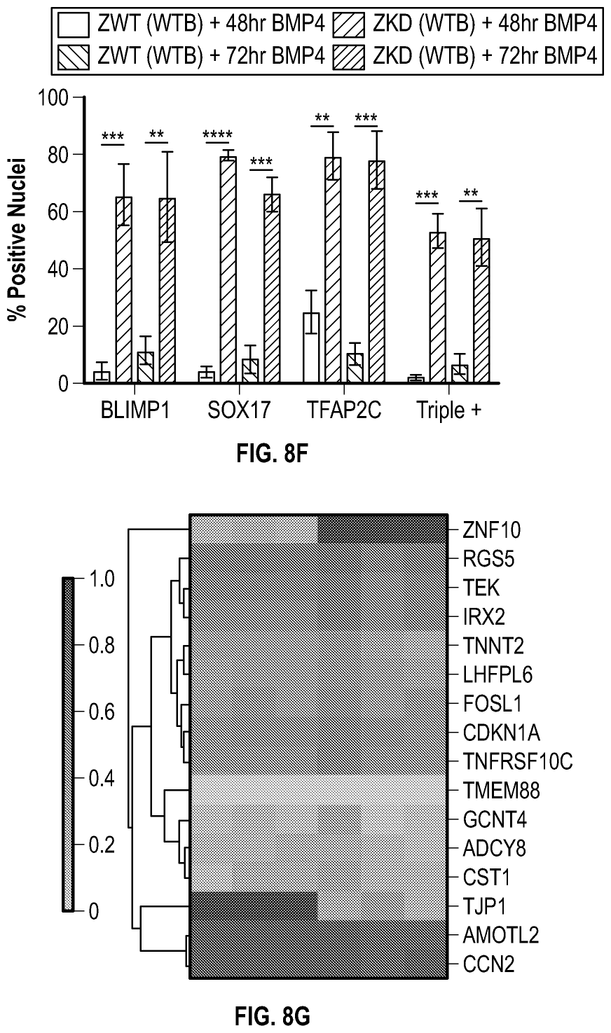

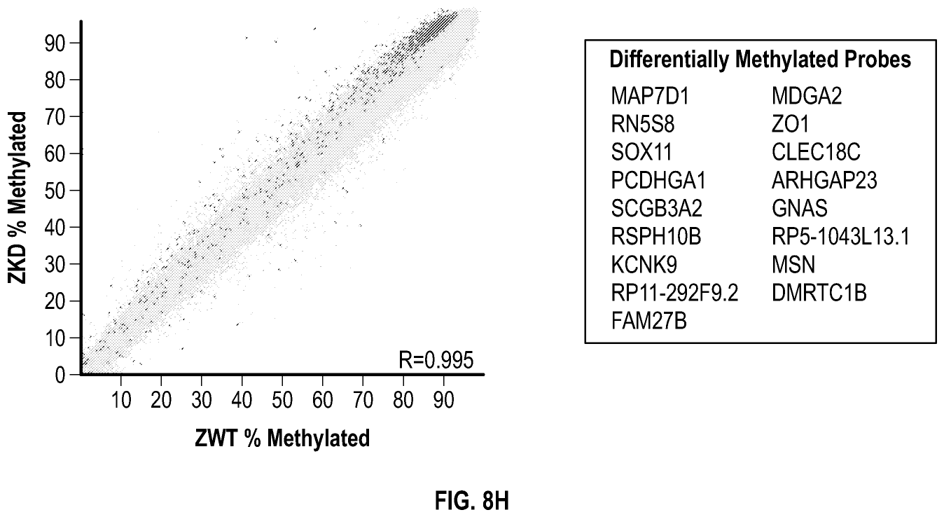

Similarly, unbiased clustering of the top 16 differentially expressed genes between ZO1 wild type and ZO1 knockdown revealed significant increases in NANOS3, SOX17, and WNT3 (FIG. 7F), genes that when expressed together are associated with the human PGC specification program (Irie et al., Cell 160, 253-268 (2015)). Subsequent immunofluorescence staining for PGC markers BLIMP1, TFAP2C, and SOX17 at 48 hours showed increased expression of these markers in ZO1 knockdown colonies at 48 hours compared with the ZO1 wild type controls (FIG. 7G). This phenotype can also be observed outside of the colony format at 48 hours. By 72 hours, clear triple positive expression of BLIMP1/TFAP2C/SOX17 can be seen in the majority of ZO1 knockdown cells (FIG. 8A-8B) in monolayer culture, a phenotype that is also observed in the WTB ZO1 knockdown hPSC line (FIG. 8E-8F). Together, these results indicate that disrupting tight junction “stability” in the presence of BMP4 dramatically augments cell receptiveness to signals needed for PGCLC emergence.

### Example 8: Decoupling Signaling and Structural Changes in ZKD PGCLCs

Upon the discovery of a nascent PGCLC population within the ZO1 knockdown colonies, the inventors sought to decouple the effects of structural changes due to tight junction instability and ubiquitous pSMAD1 activation in enabling this PGCLC population to emerge. Two papers describe different protocols for generating human PGCLCs (Irie et al., Cell 160, 253-268 (2015); Sasaki et al. Cell Stem Cell 17, 178-194 (2015)). In the first protocol by Sasaki et al., hPSCs were pre-induced into an incipient mesoderm-like (iMeLC) state that renders the cells poised for PGCLC specification. In the second protocol by Irie et al., hPSCs are first reset from a primed to a naïve pluripotency state, as primed hPSCs are thought to have lost the developmental potential to generate PGCLCs. Without iMeLC or naïve pluripotency pre-induction, both protocols failed to efficiently generate PGCLCs, providing only about 1-2% efficiency of generating PGCLCs.

However, using the differentiation methods described herein, ZO1 knockdown cells do not undergo any form of pre-induction yet are able to produce a robust PGCLC population.

Two possibilities potentially explain this PGCLC specification bias: 1) ZO1 knockdown is causing a change in pluripotent ground state (to a naïve-like or iMeLC-like state), or 2) signaling changes caused by ZO1 knockdown recapitulate in vivo PGC specification, and are sufficient to drive PGCLC differentiation in vitro.

The inventors first characterized pluripotency in ZO1 wild type and ZO1 knockdown cells in the absence of BMP4. RNA sequencing showed that aside from ZO1 and ZNF10 (which is part of the CRISPRi machinery), few genes are both significantly and substantially differentially expressed between ZO1 wild type and ZO1 knockdown cells (FIG. 8G), and no significant changes are shown in major canonical pluripotency markers (FIG. 8C). Whole genome bisulfite sequencing shows that while several probes are differentially methylated (FIG. 8D, 8H), there are no global changes in methylation of probes between ZO1 wild type and ZO1 knockdown cells, which would be expected if a resetting process occurred. GO analysis also did not reveal any significant links between genes with methylated probes. Together, these data indicate that the transcriptome and methylome are not greatly affected and there is no observable change in ground state that explains ZO1 knockdown predisposition to PGCLC lineages.

Next the inventors tested the hypothesis that ZO1 knockdown cells are predisposed to PGCLC fates because, unlike ZO1 wild type cells which undergo NOGGIN-related BMP4-pathway inhibition at later timepoints, ZO1 knockdown cells are able to maintain BMP4-pathway activation.

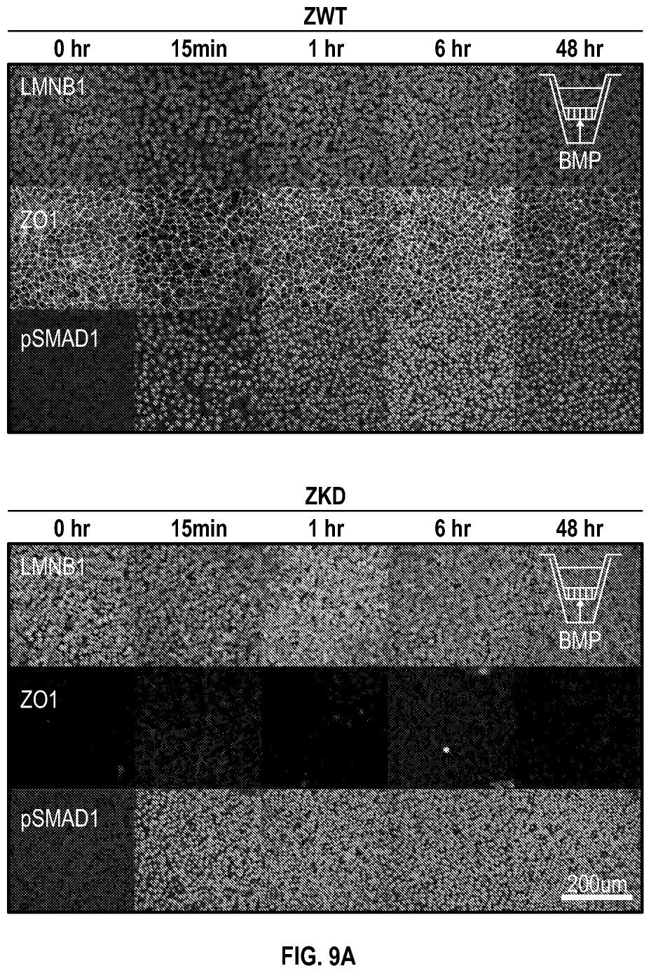

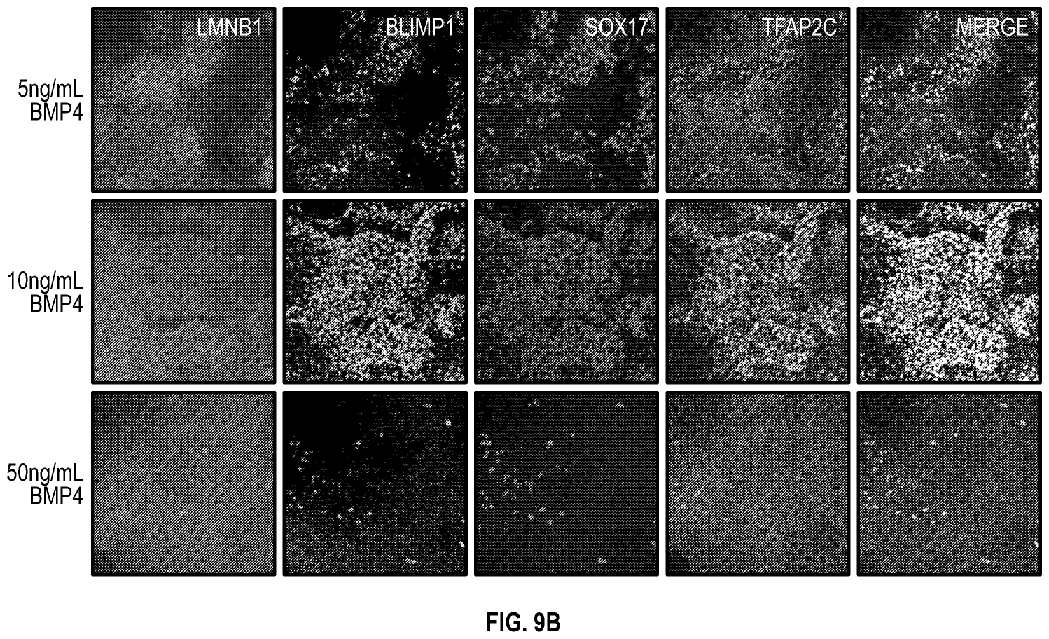

To decouple changes in signaling from potential structural changes that result from ZO1 knockdown, the inventors designed experiments to recapitulate the pSMAD1 signaling dynamics in hPSCs without ZO1 knockdown. ZO1 wild type cells were grown on a transwell membrane where both the apical and basolateral sides were exposed to the media. As described, bi-directional stimulation of hPSCs with BMP4 resulted in ubiquitous and sustained activation of pSMAD1 over the course of 48 hours, much like when ZO1 knockdown cells are stimulated in standard culture (FIG. 9A). RNA sequencing of stimulated ZO1 wild type and ZO1 knockdown cells grown on transwells showed remarkable similarities in marker expression between the two samples, demonstrating that most of the observed changes in cell fate are a direct result of increased signal pathway activation. The total number of differentially expressed genes between ZO1 wild type and ZO1 knockdown samples was significantly higher in standard culture (3150) versus in transwell (35) culture, highlighting the magnitude of the expression changes dependent solely on changes in pSMAD1 signaling. Of these 35 genes, unbiased clustering and GO analysis demonstrated that ZO1 knockdown cells still have a slight bias towards mesendodermal lineages, as illustrated in Table 7 below.

TABLE 7
Gene Sets Enriched in ZO1 Knockdown Cells

| Gene-set Enriched GO Terms | FDR |
|---|---|
| Cluster 2: | |
| Primitive streak formation | 4.62E−02 |
| Cluster 3: | |
| Embryonic foregut morphogenesis | 7.50E−04 |
| Cellular response to erythropoietin | 2.93E−02 |

Interestingly, neither ZO1 wild type nor ZO1 knockdown cells grown on transwell membranes and treated for 48 hours with BMP4 (50 ng/ml) were as predisposed to PGCLC fates as was seen for ZO1 knockdown cells on standard plates. The hypothesized that this was a result of too much signal from bi-directional stimulation on the transwell. Decreasing the BMP4 concentration to 10 ng/mL resulted in robust and ubiquitous PGCLC differentiation of ZO1 wild type cells on the transwell membranes (FIG. 9B). Taken together, these results indicate that changes in cell identity in the absence of ZO1, and specifically the emergence of a PGCLC population, are largely due to increased susceptibility to BMP4 signaling.

### REFERENCES

1. Tam, P. P. L. & Loebel, D. A. F. Gene function in mouse embryogenesis: get set for gastrulation. 8, 368-381 (2007).
2. Rossant, J. & Tam, P. P. L. Blastocyst lineage formation, early embryonic asymmetries and axis patterning in the mouse. 713, 701-713 (2009).
3. Tam, P. P. L. & Behringer, R. R. Mouse gastrulation: the formation of a mammalian body plan. 68, 3-25 (1997).
4. Farquhar, M. G. & Palade, G. Junctional complexes in various epithelia. J. Cell Biol. 375-412 (1963).
5. Claude, P. & Goodenough, D. A. Fracture faces of zonulae occludentes from ‘tight’ and ‘leaky’ epithelia. 58, 390-400 (1973).
6. Zihni, C., Mills, C., Matter, K. & Balda, M. S. Tight junctions: from simple barriers to multifunctional molecular gates. Nat. Rev. 17, 564-580 (2016).
7. Murphy, S. J. et al. Differential Trafficking of Transforming Growth Factor-Receptors and Ligand in Polarized Epithelial Cells. Mol. Biol. Cell 15, 2853-2862 (2004).
8. Yin, X. et al. Basolateral delivery of the type I transforming growth factor beta receptor is mediated by a dominant-acting cytoplasmic motif. Mol. Biol. Cell 28, 2701-2711 (2017).
9. Phan-Everson, T. et al. Differential compartmentalization of BMP4/NOGGIN requires NOGGIN trans-epithelial transport. Dev. Cell 56, 1930-1944.e5 (2021).
10. Zhang, Z., Zwick, S., Loew, E., Grimley, J. S. & Ramanathan, S. Mouse embryo geometry drives formation of robust signaling gradients through receptor localization. Nat. Commun. 10, (2019).
11. Xiang, L. et al. A developmental landscape of 3D-cultured human pre-gastrulation embryos. Nature 577, (2020).
12. Ben-Haim, N. et al. The Nodal Precursor Acting via Activin Receptors Induces Mesoderm by Maintaining a Source of Its Convertases and BMP4. Dev. Cell 11, 313-323 (2006).
13. Arnold, S. J. & Robertson, E. J. Making a commitment: Cell lineage allocation and axis patterning in the early mouse embryo. Nat. Rev. Mol. Cell Biol. 10, 91-103 (2009)
14. Zimmerman, L. B., Jesu, M. De & Harland, R. M. The Spemann Organizer Signal noggin Binds and Inactivates Bone Morphogenetic Protein 4. 86, 599-606 (1996).
15. Bardot, E. S. & Hadjantonakis, A. Mechanisms of Development Mouse gastrulation: Coordination of tissue patterning, specification and diversification of cell fate Post-implantation Pre-streak Mid gastrulation. Mech. Dev. 163, 103617 (2020).
16. Krtolica, A., Genbacev, O., Escobedo, C., Zdravkovic, T., Nordstrom, A., Vabuena, D., Nath, A., Simon, C., Mostov, K. and Fisher, S. J. Disruption of Apical-Basal Polarity of Human Embryonic Stem Cells Enhances Hematoendothelial Differentiation. Stem Cells 25, 2215-2223 (2007).
17. Warmflash, A., Sorre, B., Etoc, F., Siggia, E. D. & Brivanlou, A. H. A method to recapitulate early embryonic spatial patterning in human embryonic stem cells. Nat. Methods 11, 847-854 (2014).
18. Deglincerti, A. et al. Self-organization of human embryonic stem cells on micropatterns. Nat. Protoc. 11, 2223-2232 (2016).
19. Minn, K. T. et al. High-Resolution Transcriptional and Morphogenetic Profiling of Cells from Micropatterned Human Embryonic Stem Cell Gastruloid Cultures. SSRN Electron. J. (2020). doi:10.2139/ssrn.3528686
20. Etoc, F. et al. A Balance between Secreted Inhibitors and Edge Sensing Controls Gastruloid Self-Organization. Dev. Cell 39, 302-315 (2016).
21. Tewary, M. et al. A stepwise model of Reaction-Diffusion and Positional-Information governs self-organized human peri-gastrulation-like patterning. Development dev.149658 (2017). doi:10.1242/dev.149658
22. Martyn, I., Brivanlou, A. H. & Siggia, E. D. A wave of WNT signalling balanced by secreted inhibitors controls primitive streak formation in micropattern colonies of human embryonic stem cells. Development dev. 172791 (2019). doi:10.1242/dev.172791
23. Muncie, J. M. et al. Mechanical Tension Promotes Formation of Gastrulation-like Nodes and Patterns Mesoderm Specification in Human Embryonic Stem Cells. Dev. Cell 1-16 (2020). doi:10.1016/j.devcel.2020.10.015
24. Fanning, A. S. & Anderson, J. M. Zonula Occludens-1 and -2 Are Cytosolic Scaffolds That Regulate the Assembly of Cellular Junctions. Ann. N. Y. Acad. Sci. Vol. 1165, 113-120 (2009).
25. Mcneil, E., Capaldo, C. T. & Macara, I. G. Zonula Occludens-1 Function in the Assembly of Tight Junctions in Madin-Darby Canine Kidney Epithelial Cells □. 17, 1922-1932 (2006).
26. Libby, A. R. G. et al. Spatiotemporal mosaic patterning of pluripotent stem cells using CRISPR interference. bioRxiv 1-23 (2018). doi: https://doi.org/10.1101/252189
27. Joy, D. A., Libby, A. R. G. & McDevitt, T. C. Deep neural net tracking of human pluripotent stem cells reveals intrinsic behaviors directing morphogenesis. Stem Cell Reports 16, 1317-1330 (2021).
28. Gunne-Braden, A. et al. GATA3 Mediates a Fast, Irreversible Commitment to BMP4-Driven Differentiation in Human Embryonic Stem Cells. Cell Stem Cell 26, 693-706.e9 (2020)
29. Nallet-Staub, F. et al. Cell Density Sensing Alters TGF-β Signaling in a Cell-Type-Specific Manner, Independent from Hippo Pathway Activation. Dev. Cell 32, 640-651 (2015)
30. Smith, Q. et al. Cytoskeletal tension regulates mesodermal spatial organization and subsequent vascular fate. Proc. Natl. Acad. Sci. U.S.A. 115, 8167-8172 (2018).
31. Manfrin, A. et al. Engineered signaling centers for the spatially controlled patterning of human pluripotent stem cells. Nat. Methods 16, 640-648 (2019).
32. Kim, Y. et al. Cell position within human pluripotent stem cell colonies determines apical specialization via an actin cytoskeleton-based mechanism. Stem Cell Reports 17, 68-81 (2022).
33. Krtolica, A. et al. Disruption of Apical-Basal Polarity of Human Embryonic Stem Cells Enhances Hematoendothelial Differentiation. Stem Cells 25, 2215-2223 (2007).
34. Paine-Saunders, S., Viviano, B. L., Economides, A. N. & Saunders, S. Heparan sulfate proteoglycans retain Noggin at the cell surface. A potential mechanism for shaping bone morphogenetic protein gradients. J. Biol. Chem. 277, 2089-2096 (2002).
35. Granés, F., Ureña, J. M., Rocamora, N. & Vilaró, S. Ezrin links syndecan-2 to the cytoskeleton. J. Cell Sci. 113, 1267-1276 (2000).
36. Yadav, S., Puri, S. & Linstedt, A. D. A Primary Role for Golgi Positioning in Directed Secretion, Cell Polarity, and Wound Healing. 20, 1728-1736 (2009).
37. Rodriguez-Boulan, E. & Macara, I. G. Organization and execution of the epithelial polarity programme. Nat. Rev. Mol. Cell Biol. 15, 225-242 (2014).
38. Turing, A. M. The chemical basis of morphogenesis. Philos, Trans. R. Soc. 37-72 (1952). doi: https://doi.org/10.1098/rstb.1952.0012
39. Wolpert, L. Positional information and the spatial pattern of cellular differentiation. J. Theor. Biol. 25, 1-47 (1969).
40. Green, J. B. A. & Sharpe, J. Positional information and reaction-diffusion: Two big ideas in developmental biology combine. Dev. 142, 1203-1211 (2015).
41. Irie, N. et al. SOX17 is a critical specifier of human primordial germ cell fate. Cell 160, 253-268 (2015).
42. Sasaki, K. et al. Robust In Vitro Induction of Human Germ Cell Fate from Pluripotent Stem Cells. Cell Stem Cell 17, 178-194 (2015).
43. Larripa, K. & Gallegos, A. A mathematical model of Noggin and BMP densities in adult neural stem cells. Lett. Biomath. 4, 1-22 (2017).
44. Jones, C. M. & Smith, J. C. Establishment of a BMP-4 Morphogen Gradient by Long-Range Inhibition. 17, 12-17 (1998).
45. Farin, H. F. et al. Visualization of a short-range Wnt gradient in the intestinal stem-cell niche. Nature (2016). doi:10.1038/nature16937
46. Lawson, K. A. et al. Bmp4 is required for the generation of primordial germ cells in the mouse embryo. Genes Dev. 13, 424-436 (1999).
47. Sasaki, K. et al. The Germ Cell Fate of Cynomolgus Monkeys Is Specified in the Nascent Amnion. Dev. Cell 39, 169-185 (2016).
48. Mandegar, M. A. et al. CRISPR Interference Efficiently Induces Specific and Reversible Gene Silencing in Human iPSCs. Cell Stem Cell 18, 541-553 (2016).
49. Hookway, T. A., Butts, J. C., Lee, E., Tang, H. & McDevitt, T. C. Aggregate formation and suspension culture of human pluripotent stem cells and differentiated progeny. Methods 101, 11-20 (2016).
50. Ungrin, M. D., Joshi, C., Nica, A., Bauwens, C. & Zandstra, P. W. Reproducible, ultra high-throughput formation of multicellular organization from single cell suspension-derived human embryonic stem cell aggregates. PLOS One 3, (2008)
51. Hayama, T. et al. Generation of mouse functional oocytes in rat by xeno-ectopic transplantation of primordial germ cells. Biol. Reprod. 91, 1-9 (2014).
52. Hayashi, K., Ohta, H., Kurimoto, K., Aramaki, S. & Saitou, M. Reconstitution of the mouse germ cell specification pathway in culture by pluripotent stem cells. Cell 146, 519-532 (2011).
53. Hayashi, K. et al. Offspring from Oocytes Derived from in Vitro Primordial Germ Cell-like Cells in Mice. Science (80-.). 971, 10-15 (2012).
54. Irie et al., Germ cell specification and pluripotency in mammals: a perspective from early embryogenesis. Reprod. Med. Biol. 13, 203-215 (2014).
55. Irie et al., SOX17 is a Critical Specifier of Human Primordial Germ Cell Fate, Cell 160:253-268 (2015)
56. Mandegar et al., CRISPR interference efficiently induces specific and reversible gene silencing in human iPSCs. Cell Stem Cell 18:541-553 (2016).
57. Matoba, S. & Ogura, A. Generation of functional oocytes and spermatids from fetal primordial germ cells after ectopic transplantation in adult mice. Biol. Reprod. 84, 631-638 (2011).
58. Sasaki et al., Robust In Vitro Induction of Human Germ Cell Fate from Pluripotent Stem Cells, Cell Stem Cell 17 (2): 178-94 (2015).
59. Theunissen et al., Systematic identification of culture conditions for induction and maintenance of naive human pluripotency, Cell Stem Cell 15 (4): 471-487 (2014).
60. Qing, T. et al. Mature oocytes derived from purified mouse fetal germ cells. Hum. Reprod. 23, 54-61 (2008).
61. Zhou, Q. et al. Complete Meiosis from Embryonic Stem Cell-Derived Germ Cells in Vitro. Cell Stem Cell 18, 330-340 (2016).

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements are intended to describe and summarize various embodiments of the invention according to the foregoing description in the specification.

### Statements:

1. A system comprising pluripotent stem cells supported on a porous surface in a culture medium that contains BMP.

2. The system of statement 1, wherein the pluripotent stem cells are human pluripotent stem cells.

3. The system of statement 1 or 2, wherein the pluripotent stem cells are induced pluripotent stem cells.

4. The system of statement 1, 2 or 3, wherein the pluripotent stem cells are genetically modified.

5. The system of any one of statements 1-4, wherein the pluripotent stem cells are genetically modified to correct a genetic defect.

6. The system of any one of statements 1-5, wherein the pluripotent stem cells are genetically modified to reduce the expression or function of an endogenous tight junction gene.

7. The system of statement 6, wherein the tight junction gene is at least one endogenous zonula occludens-1 (ZO1), zonula occludens-2 (ZO2), zonula occludens-3 (ZO3), OCLN, CLDN2, CLDN5, CLDN6, or CLDN7 gene.

8. The system of any one of statements 1-7, wherein the porous surface has pores that the cells cannot pass through.

9. The system of any one of statements 1-8, wherein the porous surface has pores of about 0.4 μm to about 8.0 μm in diameter.

10. The system of any one of statements 1-9, wherein the porous surface is a membrane.

11. The system of any one of statements 1-10, wherein the porous surface is an insert of a transwell plate.

12. The system of any one of statements 1-11, wherein the system comprises a transwell plate.

13. The system of any one of statements 1-12, wherein the BMP is BMP2, BMP4, or a combination thereof.

14. The system of any one of statements 1-13, which comprises an apical compartment and a basolateral compartment.

15. The system of any one of statements 1-14 wherein the pluripotent stem cells are within or receive BMP from a basolateral compartment.

16. The system of any one of statements 1-15, wherein the BMP is at a concentration of at least 0.1 ng/ml, or at least 1 ng/ml, or at about 2 ng/ml or at least 5 ng/ml, or at least 10 ng/ml, or at least 20 ng/ml, or at least 25 ng/ml, or at least 30 ng/ml, or at least 35 ng/ml, or at least 40 ng/ml, or at least 50 ng/ml.

17. The system of any one of statements 1-16, wherein the BMP is at a concentration of less than 200 ng/ml, or less than 150 ng/ml, or less than 100 ng/ml, or less than 75 ng/ml, or less than 60 ng/ml.

18. The system of any one of statements 1-17, wherein the porous surface is conditioned with extracellular matrix protein prior to seeding the pluripotent stem cells on the porous surface.

19. The system of statement 18, wherein the extracellular matrix protein is removed from the porous surface prior to seeding the pluripotent stem cells on the porous surface.

20. The system of any one of statements 1-19, wherein the pluripotent stem cells are incubated with a ROCK inhibitor prior to seeding the pluripotent stem cells on the porous surface.

21. The system of any one of statements 1-20, further comprising at least one primordial germ cell.

22. The system of any one of statements 1-21, further comprising a population of primordial germ cells.

23. A method comprising inhibiting or bypassing tight junction formation in a population of pluripotent stem cells to generate a modified cell population, and contacting the tight-junction modified cell population with BMP.

24. The method of statement 23, wherein inhibiting or bypassing tight junction formation comprises:
a. incubating the population of pluripotent stem cells on a porous surface to bypass apical tight junctions;
b. contacting the population of pluripotent stem cells with one or more inhibitory nucleic acids that bind one or more tight junction nucleic acids (one or more tight junction mRNA or DNA);
c. contacting the population of pluripotent stem cells with one or more CRISPRi ribonucleoprotein (RNP) complexes targeted to one or more tight junction gene;
d. contacting the population of pluripotent stem cells with one or more expression vectors or virus-like particles (VLP) encoding one or more guide RNAs that can bind one or more tight junction gene; and
e. combinations thereof.

25. The method of statement 24, wherein the porous surface has pores that the cells cannot pass through.

26. The method of statement 24 or 25, wherein the porous surface has pores of about 0.4 μm to about 8.0 μm in diameter.

27. The method of statement 24, 25 or 26, wherein the porous surface is a membrane.

28. The method of any one of statements 24-27, wherein the porous surface is an insert of a transwell plate.

29. The method of any one of statements 28, wherein the transwell plate comprises an apical compartment and a basolateral compartment.

30. The method of statement 29, wherein the basolateral compartment comprises culture medium comprising BMP.

31. The method of any one of statements 24-30, wherein the porous surface is conditioned with extracellular matrix protein prior to seeding the pluripotent stem cells on the porous surface.

32. The method of statement 31, wherein the extracellular matrix protein is removed from the porous surface prior to seeding the pluripotent stem cells on the porous surface.

33. The method of any one of statements 24-32, wherein the inhibitory nucleic acids that bind one or more tight junction nucleic acids comprise one or more short interfering RNA (siRNA), IRNA, antisense nucleic acid, or a combination thereof.

34. The method of any one of statements 24-33, wherein the population of pluripotent stem cells contacted with one or more CRISPRi ribonucleoprotein (RNP) complexes comprises pluripotent stem cells that express a cas nuclease.

35. The method of any one of statements 23-34, wherein inhibiting the tight junction formation comprises incubating the population of pluripotent stem cells with a chelator or chemical inhibitor.

36. The method of statement 35, wherein the chelator or chemical inhibitor is ethylenediaminetetraacetic acid (EDTA), ethylene glycol-bis (β-aminoethyl ether)-N,N,N′,N′-tetraacetic acid (EGTA), dimercaptosuccinic acid, dimercaprol, genistein, 1-tert-Butyl-3-(4-chlorophenyl)-1H-pyrazolo[3,4-d]pyrimidin-4-amine (PP2), glycyrrhizin, or a combination thereof.

37. The method of any one of statements 23-36, wherein inhibiting the tight junction formation comprises incubating the population of pluripotent stem cells with PTPN1, acetylaldehyde, genistein, protein phosphatase 2 (PP2), Clostridium perfringens enterotoxins (and their derived mutants), monoclonal antibodies against Claudin-1 (75A, OM-7D3-B3, 3A2, 6F6), monoclonal antibodies against Claudin-6 (IMAB027), Claudin-2 (1A2), monoclonal antibodies against Claudin-5 (R9, R2, 2B12), monoclonal antibodies against Occludin (1-3, 67-2), and combinations thereof.

38. The method of any one of statements 23-37, wherein inhibiting the tight junction formation comprises inhibiting expression or function of at least one endogenous zonula occludens-1 (ZO1), zonula occludens-2 (ZO2), zonula occludens-3 (ZO3), OCLN, CLDN2, CLDN5, CLDN6, or CLDN7 gene.

39. The method of any one of statements 23-38, wherein inhibiting the tight junction formation comprises inhibiting expression or function of at least one endogenous zonula occludens-1 (ZO1) allele.

40. The method of any one of statements 23-39, wherein the population of pluripotent stem cells and/or the tight-junction modified cell population are incubated in a culture medium comprising a ROCK inhibitor.

41. The method of any one of statements 23-40, wherein the pluripotent stem cells are human pluripotent stem cells.

42. The method of any one of statements 23-41, wherein the pluripotent stem cells are autologous or allogenic to a selected subject.

43. The method of statement 42, wherein the selected subject is a bird or mammal.

44. The method of statement 42 or 43 wherein the selected subject is a domesticated animal, a zoo animal, an endangered animal (e.g., an animal on an endangered species list), or a human.

45. The method of any one of statements 23-44, wherein the pluripotent stem cells are induced pluripotent stem cells.

46. The method of any one of statements 23-45, wherein the pluripotent stem cells are genetically modified.

47. The method of any one of statements 23-46, wherein the pluripotent stem cells are genetically modified to correct a genetic defect.

48. The method of any one of statements 23-47, wherein the pluripotent stem cells are genetically modified to reduce the expression or function of an endogenous tight junction gene.

49. The method of any one of statements 23-48, wherein the BMP is BMP2, BMP4, or a combination thereof.

50. The method of any one of statements 23-49, wherein the BMP is at a concentration of at least 0.1 ng/ml, or at least 1 ng/ml, or at about 2 ng/ml or at least 5 ng/ml, or at least 10 ng/ml, or at least 20 ng/ml, or at least 25 ng/ml, or at least 30 ng/ml, or at least 35 ng/ml, or at least 40 ng/ml, or at least 50 ng/ml.

51. The method of any one of statements 23-50, wherein the BMP is at a concentration of less than 200 ng/ml, or less than 150 ng/ml, or less than 100 ng/ml, or less than 75 ng/ml, or less than 60 ng/ml.

52. The method of any one of statements 23-51, further comprising harvesting at least one primordial germ cell from the culture medium containing BMP.

53. The method of any one of statements 28-52, further comprising differentiating at least one primordial germ cell into one or more mature germ cells.

54. The method of any one of statements 28-52, further comprising administering or implanting at least one primordial germ cell into a selected subject.

55. A modified pluripotent stem cell comprising a knockdown or knockout of an endogenous zonula occludens-1 (ZO1), zonula occludens-2 (ZO2), zonula occludens-3 (ZO3), OCLN, CLDN2, CLDN5, CLDN6, or CLDN7 gene.

56. A population of modified pluripotent stem cells, each primordial germ cell comprising a knockdown or knockout of an endogenous zonula occludens-1 (ZO1), zonula occludens-2 (ZO2), zonula occludens-3 (ZO3), OCLN, CLDN2, CLDN5, CLDN6, or CLDN7 gene.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a nucleic acid” or “a protein” or “a cell” includes a plurality of such nucleic acids, proteins, or cells (for example, a solution or dried preparation of nucleic acids or expression cassettes, a solution of proteins, or a population of cells), and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

1. A method comprising inhibiting or bypassing tight junction formation in a population of pluripotent stem cells to generate a modified cell population, and contacting the modified cell population with BMP.

2. The method of claim 1, wherein inhibiting or bypassing tight junction formation comprises:
a. incubating the population of pluripotent stem cells on a porous surface to bypass apical tight junctions;
b. contacting the population of pluripotent stem cells with one or more inhibitory nucleic acids that bind one or more tight junction nucleic acids;
c. contacting the population of pluripotent stem cells with one or more CRISPRi ribonucleoprotein (RNP) complexes targeted to one or more tight junction gene;
d. contacting the population of pluripotent stem cells with one or more expression vectors or virus-like particles (VLP) encoding one or more guide RNAs that can bind one or more tight junction gene;
e. incubating the population of pluripotent stem cells with a chelator or inhibitor; and
f. combinations thereof.

3. The method of claim 2, wherein the porous surface is a membrane or an insert of a transwell plate.

4. (canceled)

5. (canceled)

6. (canceled)

7. The method of claim 2, wherein the inhibitory nucleic acids that bind one or more tight junction nucleic acids comprise one or more short interfering RNA (siRNA), iRNA, antisense nucleic acid, or a combination thereof.

8. The method of claim 2, wherein the population of pluripotent stem cells contacted with one or more CRISPRi ribonucleoprotein (RNP) complexes comprises pluripotent stem cells that express a cas nuclease.

9. The method of claim 2, wherein the chelator or inhibitor is ethylenediaminetetraacetic acid (EDTA), ethylene glycol-bis (β-aminoethyl ether)-N,N,N′,N′-tetraacetic acid (EGTA), dimercaptosuccinic acid, dimercaprol, genistein, 1-tert-Butyl-3-(4-chlorophenyl)-1H-pyrazolo[3,4-d]pyrimidin-4-amine (PP2), glycyrrhizin, or a combination thereof.

10. The method of claim 1, wherein inhibiting or bypassing the tight junction formation comprises inhibiting expression or function of at least one endogenous zonula occludens-1 (ZO1), zonula occludens-2 (ZO2), zonula occludens-3 (ZO3), OCLN, CLDN2, CLDN5, CLDN6, or CLDN7 gene.

11. (canceled)

12. The method of claim 1, wherein the population of pluripotent stem cells and/or the modified cell population are incubated in a culture medium comprising a ROCK inhibitor.

13. (canceled)

14. (canceled)

15. (canceled)

16. The method of claim 1, wherein the pluripotent stem cells are genetically modified.

17. (canceled)

18. The method of claim 1, wherein the pluripotent stem cells are genetically modified to reduce the expression or function of an endogenous tight junction gene.

19. The method of claim 1, wherein the BMP is BMP2, BMP4, or a combination thereof.

20. (canceled)

21. (canceled)

22. The method of claim 1, further comprising harvesting at least one primordial germ cell from the culture medium containing BMP.

23. The method of claim 22, further comprising differentiating at least one primordial germ cell into one or more mature germ cells.

24. (canceled)

25. The method of claim 22, further comprising administering or implanting at least one primordial germ cell into a selected subject.

26. A system comprising pluripotent stem cells supported on a porous surface in a culture medium that contains BMP, wherein the porous surface has pores that the cells cannot pass through.

27. The system of claim 26, wherein the porous surface is a membrane.

28. (canceled)

29. The system of claim 26, wherein the pluripotent stem cells are genetically modified.

30. (canceled)

31. The system of claim 26, which reduces expression or function of at least one tight junction gene.

32. (canceled)

33. The system of claim 26, wherein the BMP is BMP2, BMP4, or a combination thereof.

34. (canceled)

35. (canceled)

36. The system of claim 26, further comprising at least one primordial germ cell.

37. (canceled)

38. A modified pluripotent stem cell comprising knockdown or knockout of an endogenous zonula occludens-1 (ZO1), zonula occludens-2 (ZO2), zonula occludens-3 (ZO3), OCLN, CLDN2, CLDN5, CLDN6, or CLDN7 gene.

39. (canceled)

40. (canceled)
